SEQUENCE LISTING (3 sequences)

SEQ ID NO: 1 - protein, 31 aa, Homo sapiens
HAEGTFTSDVSSYLEGQAAKEFIAWLVKGRG

SEQ ID NO: 2 - protein, 30 aa, Homo sapiens
HAEGTFTSDVSSYLEGQAAKEFIAWLVKGR

SEQ ID NO: 3 - protein, 39 aa, Homo sapiens
HGEGTFTSDLSKQMEEEAVRLFIEWLKNGGPSSGAPPPS

(19) United States
(12) Patent Application Publication
(10) Pub. No.: US 2008/0260838 A1
(43) Pub. Date: Oct. 23, 2008

(54) GLUCAGON-LIKE PEPTIDE 1 (GLP-1) PHARMACEUTICAL FORMULATIONS

(75) Inventors: Mark J. Hokenson, Valencia, CA (US); David Brandt, Valencia, CA (US); Mark King, Pasadena, CA (US); Stephanie Greene, Ventura, CA (US); Mary Faris, Los Angeles, CA (US); Keith Oberg, Valencia, CA (US); Wayman Wendell Cheatham, Columbia, MD (US); Cohava Gelber, Manassas, VA (US); Andrea Leone-Bay, Ridgefield, CT (US)

Correspondence Address: KIRKPATRICK & LOCKHART PRESTON GATES ELLIS LLP, 1900 MAIN STREET, SUITE 600, IRVINE, CA, 92614-7319, US

(73) Assignee: MANNKIND CORPORATION, Valencia, CA (US)

(21) Appl. No.: 11/735,957

(22) Filed: Apr. 16, 2007

Related U.S. Application Data
(63) Continuation-in-part of application No. 10/632,878, filed on Aug. 1, 2003.
(60) Provisional application No. 60/744,882, filed on Apr. 14, 2006.

(51) Int. Cl. A61K 9/14; A61K 38/16; A61P 3/10; A61P 3/04; A61P 35/00

(52) U.S. Cl. 424/489; 514/12

(57) ABSTRACT

A composition is disclosed comprising glucagon-like peptide 1 (GLP-1) particles in combination with diketopiperazine (DKP) that is stable both in vitro and in vivo. The composition has utility as a pharmaceutical formulation for treating diseases such as diabetes, cancers, and obesity but is not limited to such diseases or conditions. In particularly, the composition has utility as a pharmaceutical formulation for pulmonary delivery.

### CROSS REFERENCE TO RELATED APPLICATIONS

This application is a continuation-in-part of U.S. application Ser. No. 10/632,878, filed Jul. 22, 2003 and claims the benefit under 35 U.S.C. §119(e) to U.S. Provisional Application No. 60/744,882, filed on Apr. 14, 2006. Each of the above-mentioned priority applications is hereby incorporated by reference in its entirety.

### FIELD OF THE INVENTION

The present invention relates to the field of pharmaceutical formulations. The present invention discloses dry powder formulations comprising diketopiperazine (DKP) particles in combination with glucagon-like peptide 1 (GLP-1). The present invention has utility as a pharmaceutical formulation for treating diseases such as diabetes, cancers, and obesity but is not limited to such diseases. More particularly, the present invention has utility as a pharmaceutical formulation for pulmonary delivery.

### BACKGROUND TO THE INVENTION

Glucagon-like peptide 1 (GLP-1) as disclosed in the literature is a 30 or 31 amino acid incretin, released from the intestinal endocrine L-cells in response to fat, carbohydrate ingestion, and protein from a meal. Secretion of this peptide hormone is found to be impaired in individuals with type 2 diabetes mellitus making it a potential candidate for the treatment of this and other related diseases.

In the non-disease state, GLP-1 is secreted from the intestinal L-cell in response to orally ingested nutrients, (particularly sugars), stimulating meal-induced insulin release from the pancreas, inhibiting glucagon release from the liver, as well as other effects on the gastrointestinal tract, and brain. GLP-1 effect in the pancreas is glucose dependent, minimizing the risk of hypoglycemia during exogenous peptide administration. GLP-1 also promotes all steps in insulin biosynthesis and directly stimulates β-cell growth and survival as well as β-cell differentiation. The combination of these effects results in increased β-cell mass. Furthermore, GLP-1 receptor signaling results in a reduction of β-cell apoptosis, which further contributes to increased β-cell mass.

In the gastrointestinal tract, GLP-1 inhibits GI motility, increases the secretion of insulin in response to glucose, and decreases the secretion of glucagon, thereby contributing to a reduction of glucose excursion. Central administration of GLP-1 has been shown to inhibit food intake in rodents, suggesting that peripherally released GLP-1 may directly affect the brain. This is feasible since it has been shown that circulating GLP-1 can access GLP-1 receptors in certain brain areas; namely the subformical organ and the area postrema. These areas of the brain are known to be involved in the regulation of appetite and energy homeostasis. Interestingly, gastric distension activates GLP-1 containing neurons in the caudal nucleus of the solitary tract, predicting a role for centrally expressed GLP-1 as an appetite suppressant. These hypotheses are supported by studies employing the GLP-1 receptor antagonist, exendin (9-39) where opposite effects were seen. In humans, administered GLP-1 has a satiating effect (Verdich et al., 2001), and when given by continuous subcutaneous infusion over a 6 weeks regime, diabetics exhibited a reduction in appetite, which led to significant reductions in body weight (Zander et al., 2002).

GLP-1 has also been shown to be effective in patients with type 2 diabetes, increasing insulin secretion and normalizing both fasting and postprandial blood glucose when given as a continuous intravenous infusion (Nauck et al., 1993). In addition, infusion of GLP-1 has been shown to lower glucose levels in patients previously treated with non-insulin oral medication and in patients requiring insulin therapy after failure on sulfonylurea therapy (Nauck et al., 1993). However, the effects of a single subcutaneous injection of GLP-1 provided disappointing results, as is noted in the art and discussed herein below. Although high plasma levels of immunoreactive GLP-1 were achieved, insulin secretion rapidly returned to pretreatment values and blood glucose concentrations were not normalized (Nauck et al., 1996). Only when repeated subcutaneous administrations were performed was the effect on fasting blood glucose comparable to intravenous administration (Nauck et al., 1996). Continuous subcutaneous administration for 6 weeks was shown to reduce fasting and postprandial glucose concentrations, and lower HbA1c levels (Zander et al., 2002). The short-lived effectiveness of single subcutaneous injections of GLP-1 was related to its circulatory instability. It was shown that GLP-1 was metabolized by plasma in vitro and that the enzyme dipeptidyl peptidase-IV (DPP-IV) was responsible for this degradation (Mentlein et al., 1993).

With the physiological significance of GLP-1 in diabetes and the demonstration that exogenous GLP-1 is rapidly amino-terminally degraded in both healthy and type 2 diabetic subjects, many studies have addressed the possibility of manipulating the in vivo stability of GLP-1 as a novel approach to an antidiabetic agent for the treatment of diabetes (Deacon et al., 2004). Two separate approaches have been pursued: 1) the development of analogs of GLP-1 that are not susceptible to enzymatic degradation and 2) the use of selective enzyme inhibitors to prevent in vivo degradation and enhance levels of the intact, biologically active peptides. Long-acting GLP-1 analogs (e.g., Liraglutide (Novo Nordisk, Copenhagen, Denmark)); exenatide (exendin-4; Byetta®) (Amylin Inc., San Diego, Calif.) and (exenatide-LAR, Eli Lilly, Indianapolis, Ind.)) that are resistant to degradation, called “incretin mimetics,” have been investigated in clinical trials. Dipeptidyl peptidase IV inhibitors (e.g., Vildagliptin (Galvus) developed by Novartis, Basel, Switzerland) and Januvia (sitagliptin) developed by Merck, Whitehouse Station, N.J.)) that inhibit the enzyme responsible for incretin degradation are also under study (Deacon et al., 2004). Thus, the multiple modes of action of GLP-1 (e.g., increased insulin release, delayed gastric emptying, and increased satiety) together with its low propensity for hypoglycemia appear to give it advantages over currently available therapies.

[However, despite these approaches/advances in GLP-1 therapy, none of the drugs currently available for diabetes are able to achieve therapeutic targets (HbA1c, fasting blood glucose, glucose excursions) in all patients and none of them are without side effects such as toxicity, hypoglycemia, weight gain, nausea and stress from vomiting. Therefore, there is still a need in the art for stable GLP-1 formulations having long term effectiveness and optimal absorption when administered as a pharmaceutical.

### SUMMARY OF THE INVENTION

Stable, inhalable glucagon-like peptide 1 (GLP-1) formulations for use as pharmaceutics are deficient in the art. In overcoming the deficiencies in the art, the present invention provides formulations of GLP-1 in combination with diketopiperazine (DKP) particles as a pharmaceutic.

Therefore, in particular embodiments of the present invention, a dry powder composition comprising a GLP-1 molecule and a diketopiperazine or a pharmaceutically acceptable salt thereof is provided. In further embodiments, the dry powder composition of the present invention comprises a GLP-1 molecule selected from the group consisting of a native GLP-1, a GLP-1 metabolite, a GLP-1 analog, a GLP-1 derivative, a dipeptidyl-peptidase-IV (DPP-IV) protected GLP-1, a GLP-1 mimetic, an exendin, a GLP-1 peptide analog, or a biosynthetic GLP-1 analog. In still yet a further embodiment of the present invention, the dry powder composition comprises a diketopiperazine having the formula 2,5-diketo-3,6-di(4-X-aminobutyl)piperazine, wherein X is selected from the group consisting of succinyl, glutaryl, maleyl, and fumaryl. In another embodiment, the dry powder composition comprises a diketopiperazine salt. In still yet another embodiment of the present invention, there is provided a dry powder composition, wherein the diketopiperazine is 2,5-diketo-3,6-di(4-fumaryl-aminobutyl)piperazine.

The present invention further contemplates a dry powder composition wherein the GLP-1 molecule is native GLP-1, or an amidated GLP-1 molecule wherein the amidated GLP-1 molecule is GLP-1 (7-36) amide.

In still yet another particular embodiment of the present invention, there is provided a process for preparing a particle comprising a GLP-1 molecule and a diketopiperazine comprising the steps of: providing a GLP-1 solution comprising a GLP-1 molecule; providing a solution of a particle-forming diketopiperazine or a suspension of particles of a diketopiperazine; and combining the GLP-1 solution with the diketopiperazine solution or suspension. In other particular embodiments of the invention, the process for preparing a particle comprising a GLP-1 molecule and a diketopiperazine further comprises removing solvent from the solution or suspension by lyophilization, filtration, or spray drying. In still yet a further embodiment, the particle of the invention is formed by removing solvent or is formed prior to removing solvent.

In an embodiment of the invention, in the process for preparing a particle having a GLP-1 molecule and a diketopiperazine, there is provided a GLP-1 molecule selected from the group consisting of a native GLP-1, a GLP-1 analog, a GLP-1 derivative, a dipeptidyl-peptidase-IV (DPP-IV) protected GLP-1, a GLP-1 mimetic, an exendin, a GLP-1 peptide analog, or a biosynthetic GLP-1 analog. In another embodiment, the process for preparing a particle having a GLP-1 molecule and a diketopiperazine comprises a diketopiperazine provided as a suspension of particles. In a further embodiment, the diketopiperazine is provided in solution and the process includes adjusting the pH of the solution to precipitate the diketopiperazine and form particles.

In other particular embodiments of the invention the GLP-1 solution is at a concentration of about 1 μg/ml-50 mg/ml, more preferably about 0.1 mg/ml-10 mg/ml. In yet another particular embodiment of the invention, the GLP-1 solution is at a concentration of about 0.25 mg/ml.

In another process for preparing a particle comprising a GLP-1 molecule and a diketopiperazine, the process further comprises adding an agent to the solution, wherein the agent is selected from salts, surfactants, ions, osmolytes, chaotropes and lyotropes, acid, base, and organic solvents. The agent promotes association between the GLP-1 and the diketopiperazine particle and also improves the stability and/or pharmacodynamics of the GLP-1 molecule. In some embodiments of the invention, the agent is a salt such as, but not limited to, sodium chloride. It is also contemplated the agent may be a surfactant such as but not limited to, Tween, Triton, pluronic acid, CHAPS, cetrimide, and Brij, H(CH₂)₇SO₄Na. The agent may be an ion, for example, a cation or anion. The agent may be an osmolyte (stabilizer), such as, but not limited to Hexylene-Glycol (Hex-Gly), trehalose, glycine, polyethylene glycol (PEG), trimethylamine n-oxide (TMAO), mannitol, and proline. The agent may be a chaotrope or lyotrope, such as, but not limited to, cesium chloride, sodium citrate, and sodium sulfate. The agent may be an organic solvent for example, an alcohol selected from methanol (MeOH), ethanol (EtOH), trifluoroethanol (TFE), and hexafluoroisopropanol (HFIP).

In another particular embodiment of the present invention, there is contemplated a process for preparing a particle comprising a GLP-1 molecule and a diketopiperazine, wherein the process comprises adjusting the pH of the particle suspension to about 4 or greater. In further embodiments of the invention, the process for preparing a particle comprises a GLP-1 molecule and a diketopiperazine, wherein the GLP-1 molecule in the particle has greater stability.

Further contemplated in the present invention is a method of administering an effective amount of a GLP-1 molecule to a subject in need thereof, comprising providing to the subject a GLP-1/diketopiperazine particle. The method of administering may be intravenously, subcutaneously, orally, nasally, buccally, rectally, or by pulmonary delivery but is not limited to such. In one embodiment, the method of administering is by pulmonary delivery. In still yet another embodiment of the invention, the method of administering comprises treating a condition or disease selected from the group consisting of diabetes, ischemia, reperfused tissue injury, dyslipidemia, diabetic cardiomyopathy, myocardial infarction, acute coronary syndrome, obesity, catabolic changes after surgery, hyperglycemia, irritable bowel syndrome, stroke, neurodegenerative disorders, memory and learning disorders, islet cell transplant and regenerative therapy.

In another embodiment of the invention, the method of administration of the GLP-1/diketopiperazine particle composition results in improved pharmacokinetic half-life and bioavailability of GLP-1.

In still yet a further particular embodiment of the present invention, there is provided a method of preparing a dry powder composition with an improved pharmacokinetic profile, comprising the steps of: providing a solution of a GLP-1 molecule; providing a particle-forming diketopiperazine; forming particles; and combining the GLP-1 and the diketopiperazine; and thereafter removing solvent by a method of drying to obtain a dry powder, wherein the dry powder has improved pharmacokinetic profile. The improved pharmacokinetic profile comprises increased half-life of GLP-1 and/or improved bioavailability of GLP-1. The increased half-life of GLP-1 is greater than or equal to 7.5 minutes.

In one embodiment of the present invention, a dry powder composition is provided comprising a GLP-1 molecule and a diketopiperazine or a pharmaceutically acceptable salt thereof. In another embodiment, the GLP-1 molecule is selected from the group consisting of native GLP-1s, GLP-1 metabolites, GLP-1 analogs, GLP-1 derivatives, dipeptidyl-peptidase-IV (DPP-IV) protected GLP-1s, GLP-1 mimetics, GLP-1 peptide analogs, or biosynthetic GLP-1 analogs.

In an embodiment of the present invention, the diketopiperazine is a diketopiperazine having the formula 2,5-diketo-3,6-di(4-X-aminobutyl)piperazine, wherein X is selected from the group consisting of succinyl, glutaryl, maleyl, and fumaryl. In another embodiment, the diketopiperazine is a diketopiperazine salt. In another embodiment, the diketopiperazine is 2,5-diketo-3,6-di(4-fumaryl-aminobutyl)piperazine.

In an embodiment of the present invention, the GLP-1 molecule is native GLP-1. In another embodiment, the GLP-1 molecule is an amidated GLP-1 molecule. In another embodiment, the amidated GLP-1 molecule is GLP-1(7-36) amide.

In one embodiment of the present invention, a process is provided for forming a particle comprising a GLP-1 molecule and a diketopiperazine comprising the steps of: providing a GLP-1 molecule; providing a diketopiperazine in a form selected from particle-forming diketopiperazine, diketopiperazine particles, and combinations thereof, and combining the GLP-1 molecule and the diketopiperazine in the form of a co-solution, wherein the particle comprising the GLP-1 molecule and the diketopiperazine is formed.

In one embodiment of the present invention, the process further comprises removing a solvent from said co-solution by lyophilization, filtration, or spray drying. In another embodiment, the particle comprising said GLP-1 molecule and the diketopiperazine is formed by removing the solvent. In another embodiment, the particle comprising the GLP-1 molecule and the diketopiperazine is formed prior to removing the solvent.

In another embodiment, the GLP-1 molecule is selected from the group consisting of a native GLP-1, a GLP-1 analog, a GLP-1 derivative, a dipeptidyl-peptidase-IV (DPP-IV) protected GLP-1, a GLP-1 mimetic, a GLP-1 peptide analog, or a biosynthetic GLP-1 analog. In another embodiment, the GLP-1 molecule is provided in the form of a solution comprising a GLP-1 concentration of about 1 μg/ml-50 mg/ml. In another embodiment, the GLP-1 molecule is provided in the form of a solution comprising a GLP-1 concentration of about 0.1 mg/ml-10 mg/ml. In another embodiment, the GLP-1 molecule is provided in the form of a solution comprising a GLP-1 concentration of about 0.25 mg/ml.

In another embodiment of the present invention, the diketopiperazine is provided in the form of a suspension of diketopiperazine particles. In another embodiment, the diketopiperazine is provided in the form of a solution comprising particle-forming diketopiperazine, the process further comprising adjusting the pH of the solution to form diketopiperazine particles. In another embodiment, the process further comprises adding an agent to said solution or suspension, wherein the agent is selected from the group consisting of salts, surfactants, ions, osmolytes, chaotropes and lyotropes, acids, bases, and organic solvents. In another embodiment, the agent promotes association between the GLP-1 molecule and the diketopiperazine particles or the particle-forming diketopiperazine. In another embodiment, the agent improves the stability or pharmacodynamics of the GLP-1 molecule. In another embodiment, the agent is sodium chloride.

In another embodiment of the present invention, the process further comprises adjusting the pH of the suspension or solution. In another embodiment, the pH is adjusted to about 4.0 or greater. In yet another embodiment, the GLP-1 molecule in the particle has greater stability than native GLP-1.

In another embodiment, the co-solution comprises a GLP-1 concentration of about 1 μg/ml-50 mg/ml. In another embodiment, the co-solution comprises a GLP-1 concentration of about 0.1 mg/ml-10 mg/ml. In another embodiment, the co-solution comprises a GLP-1 concentration of about 0.25 mg/ml.

In still yet another embodiment of the present invention, the process further comprises adding an agent to the co-solution, wherein the agent is selected from the group consisting of salts, surfactants, ions, osmolytes, chaotropes and lyotropes, acids, bases, and organic solvents. In another embodiment, the agent promotes association between the GLP-1 molecule and the diketopiperazine particles or the particle-forming diketopiperazine. In another embodiment, the agent improves the stability or pharmacodynamics of the GLP-1 molecule. In another embodiment, the agent is sodium chloride.

In another embodiment, the process further comprises adjusting the pH of the co-solution. In another embodiment, the pH is adjusted to about 4.0 or greater.

In one embodiment of the present invention, a method is provided of administering an effective amount of a GLP-1 molecule to a subject in need thereof the method comprising providing to the subject a particle comprising GLP-1 and diketopiperazine. In another embodiment, the providing is carried out intravenously, subcutaneously, orally, nasally, buccally, rectally, or by pulmonary delivery. In another embodiment, the providing is carried out by pulmonary delivery.

In another embodiment, the need comprises the treatment of a condition or disease selected from the group consisting of diabetes, ischemia, reperfused tissue injury, dyslipidemia, diabetic cardiomyopathy, myocardial infarction, acute coronary syndrome, obesity, catabolic changes after surgery, hyperglycemia, irritable bowel syndrome, stroke, neurodegenerative disorders, memory and learning disorders, islet cell transplant and regenerative therapy.

In another embodiment, the provision of the particle results in improved pharmacokinetic half-life and bioavailability of GLP-1 as compared to native GLP-1.

In one embodiment of the present invention, a method is provided of forming a powder composition with an improved GLP-1 pharmacokinetic profile, comprising the steps of: providing a GLP-1 molecule; providing a particle-forming diketopiperazine in a solution; forming diketopiperazine particles; combining the GLP-1 molecule and the solution to form a co-solution; and, removing solvent from the co-solution by spray-drying to form a powder with an improved GLP-1 pharmacokinetic profile.

In another embodiment, the improved GLP-1 pharmacokinetic profile comprises an increased GLP-1 half-life. In another embodiment, the increased GLP-1 half-life is greater than or equal to 7.5 minutes. In another embodiment, the improved GLP-1 pharmacokinetic profile comprises improved bioavailability of GLP-1 as compared to native GLP-1.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1D. Structural analysis of GLP-1 at various concentrations (pH 4, 20° C.). FIG. 1A—The far-UV circular dichroism (CD) of GLP-1 illustrates that as the concentration increases, the secondary structure of the peptide is transformed from a predominantly unstructured conformation to a helical conformation. FIG. 1B—The near-UV CD illustrates that the tertiary structure increases with increasing concentration of peptide suggesting that GLP-1 self-associates. FIG. 1C—Fluorescence emission of GLP-1 at various concentrations (pH 4, 20° C.) resulting from tryptophan excitation at 280 nm. FIG. 1D—Transmission FTIR of GLP-1 at various concentrations (pH 4, 20° C.). The amide I band at 1656 cm⁻¹ indicates that GLP-1 has a α-helical structure at concentrations ≧2 mg/mL.

FIGS. 2A-2D. Structural analysis of low concentration GLP-1 at varying ionic strength (pH 4, 20° C.). FIG. 2A—The far-UV CD of 1.0 mg/mL GLP-1 illustrates that increasing the concentration of salt converts the unordered structure of GLP-1 into more ordered α-helical structures. FIG. 2B—The near-UV CD of 1.0 mg/mL peptide demonstrates that increasing the NaCl concentration also enhances the tertiary structure of GLP-1. FIG. 2C—Intrinsic fluorescence emission of 1.0 mg/mL GLP-1 at varying NaCl concentrations (pH 4, 20° C.) following tryptophan excitation at 280 nm. At high peptide concentrations, the maxima decreases in intensity and shifts to a lower wavelength, which is indicative of a well-defined tertiary structure. FIG. 2D-Tertiary structural analysis of 10 mg/mL GLP-1 at varying ionic strength (pH 4, 20° C.). The near-UV CD spectra demonstrate that increased ionic strength enhances the tertiary structure of self-associated GLP-1.

FIGS. 3A-3B. Structural analysis of 10 mg/mL GLP-1 at various temperatures (pH 4). FIG. 3A—The near-UV CD illustrates that GLP-1 oligomers dissociate with increasing temperature. FIG. 3B—Structural analysis of 10 mg/mL GLP-1 at various temperatures (pH 4). FIG. 3C—Structural analysis of 0.05 mg/mL GLP-1 at various temperatures (pH 4). The far-UV CD illustrates that the peptide is insensitive to temperature.

FIGS. 4A-4B. Structural analysis of GLP-1 at varying pH (20° C.). FIG. 4A—The far-UV CD of 10 mg/mL GLP-1 at varying pH (20° C.). As the pH is increased, self-associated GLP-1 precipitates between pH 6.3 and 7.6 but retains a helical structure at pH 1.5 and 11.7. FIG. 4B—Enlarging the spectrum at pH 7.6 reveals that the secondary structure of GLP-1 is unordered as a result of the concentration decrease.

FIG. 5. Resistance of 1 mg/mL GLP-1 to both deamidation and oxidation as demonstrated by HPLC. Deamidation conditions were achieved by incubating GLP-1 at pH 10.5 for 5 days at 40° C. Oxidative conditions were achieved by incubating GLP-1 in 0.1% H₂O₂ for 2 hours at room temperature.

FIGS. 6A-6B. The effect of agitation on the tertiary structure of 1.5 and 9.4 mg/mL GLP-1 (pH 4). The near-UV CD (FIG. 6A) and the fluorescence emission of GLP-1 (FIG. 6B) both illustrate that the tertiary structure of GLP-1 peptide does not significantly change due to agitation. Samples were agitated for both 30 and 90 min at room temperature and the fluorescence emission spectra were collected after tryptophan excitation at 280 nm.

FIGS. 7A-7C. The effect of 10 freeze-thaw cycles on the tertiary structure of 1.6, 5.1 and 8.4 mg/mL GLP-1 (pH 4). Near-UV CD (FIG. 7A) and fluorescence emission of GLP-1 (FIG. 7B) both show that the tertiary structure of the peptide does not notably change due to multiple freeze-thaw cycles. Samples were frozen at −20° C. and were defrosted at room temperature. Fluorescence emission spectra were collected after tryptophan excitation at 280 nm. Similar experiments showing the effect of 11 freeze-thaw cycles on the secondary structure of 10 mg/mL GLP-1 (pH 4) by far-UV CD were conducted (FIG. 7C).

FIGS. 8A-8B. Salt Studies. Loading curves for GLP-1/FDKP as a function of pH and NaCl concentration (FIG. 8A). Loading was performed at 5 mg/mL FDKP and 0.25 mg/mL GLP-1. NaCl concentrations are expressed as mM. FIG. 8B—Depicts the amount of GLP-1 detected in the reconstituted FDKP-free control samples as a function of pH and NaCl concentration.

FIGS. 9A-9B. Surfactant Studies. Loading curves for GLP-1/FDKP as a function of pH and surfactant (FIG. 9A). Loading was performed at 5 mg/mL FDKP and 0.25 mg/mL GLP-1. FIG. 9B—Depicts the amount of GLP-1 detected in the reconstituted FDKP-free control samples as a function of pH and surfactant added.

FIGS. 10A-10D. Ion Studies. Loading curves for GLP-1/FDKP as a function of pH and ions. Loading was performed at 5 mg/mL FDKP and 0.25 mg/mL GLP-1 (FIGS. 10A and 11C). Ion concentrations are indicated in the legend (mM). Right-hand curves depicts the results for 1M NaCl. FIGS. 10B and 10D—Depict the amount of GLP-1 detected in the reconstituted FDKP-free control samples as a function of pH, ions and 1M NaCl.

FIGS. 11-11B. Osmolyte Studies. Loading curves for GLP-1/FDKP as a function of pH and in the presence of common stabilizers (osmolytes; FIG. 11A). Loading was performed at 5 mg/mL FDKP and 0.25 mg/mL GLP-1. FIG. 11B—Depicts the amount of GLP-1 detected in the reconstituted FDKP-free control samples as a function of pH and osmolyte. “N/A” indicates no osmolyte was present in the sample.

FIGS. 12A-12B. Chaotrope/lyotrope Studies. Loading curves for GLP-1/FDKP as a function of chaotrope or lyotrope concentration at pH 3.0 (FIG. 12A) and pH 4.0 (FIG. 12C). Loading was performed at 5 mg/mL FDKP and 0.25 mg/mL GLP-1. FIGS. 12B and 12D—Depict the amount of GLP-1 detected in the reconstituted FDKP-free control samples as a function of pH in the presence of the various chaotropes or lyotropes. “N/A” indicates no chaotropes or lyotropes were present in the sample.

FIGS. 13A-13B. Alcohol Studies. Loading curves for GLP-1/FDKP as a function of pH and alcohols. Loading was performed at 5 mg/mL FDKP and 0.25 mg/mL GLP-1. Four alcohol concentrations were evaluated for each alcohol 5%, 10%, 15%, and 20% v/v (FIG. 13A). TFE=trifluoroethanol; HFIP=hexafluoroisopropanol. FIG. 13B—Depicts the amount of GLP-1 detected for reconstituted FDKP-free control samples as a function of pH and alcohol (20%).

FIGS. 14A-14B. Loading from GLP-1/FDKP concentration studies (FIG. 14A). Loading was performed at 5 mg/mL FDKP and the GLP-1 concentration analyzed is listed in the X-axis. FIG. 14B—Scanning Electron Microscopy (SEM) images of multiple GLP-1/FDKP formulations (at 10000× magnification) depicts clusters of spherical and rod-like GLP-1/FDKP particle formulations. (Panel A) 0.5 mg/mL GLP-1 and 2.5 mg/mL FDKP; (Panel B) 0.5 mg/mL GLP-1 and 10 mg/mL FDKP; (Panel C) 0.5 mg/mL GLP-1 and 10 mg/mL FDKP in 20 mM sodium chloride, 20 mM potassium acetate and 20 mM potassium phosphate, pH 4.0; and (Panel D) 10 mg/mL GLP-1 and 50 mg/mL FDKP in 20 mM sodium chloride, 20 mM potassium acetate and 20 mM potassium phosphate, pH 4.0.

FIG. 15. Depicts the effect of stress on multiple GLP-1/FDKP formulations. The legend indicates the mass-to-mass percentage of GLP-1 to FDKP particles and the other components that were present in solution, prior to lyophilization. The samples were incubated for 10 days at 40° C.

FIGS. 16A-16C. Structure of GLP-1. FIG. 16A-Depicts the glycine-extended form of GLP-1 (SEQ ID NO. 1) and the amidated form (SEQ ID NO. 2). FIG. 16B—Inhibition of DPPIV activity by aprotinin. FIG. 16C—Inhibition of DPPIV activity by DPPIV inhibitor.

FIG. 17. Detection of GLP-1 after incubation in lung lavage fluid.

FIGS. 18A-18B. Depicts the quantitation of GLP-1 in plasma. FIG. 18A shows quantitation in 1:2 dilution of plasma. FIG. 18B shows quantitation in 1:10 dilution of plasma.

FIGS. 19A-19B. Effect of GLP-1 and GLP-1 analogs on cell survival. Effect of GLP-1 on rat pancreatic epithelial (ARIP) cell death (FIG. 19A). Annexin V staining depicting inhibition of apoptosis in the presence of GLP-1 and staurosporine (Stau) as single agents and in combination (FIG. 19B). The concentration of GLP-1 is 15 nM and the concentration of stauropsorine is 1 μM

FIG. 20. Effect of the GLP-1 analog exendin-4 on cell viability. ARIP cells were treated with 0, 10, 20 and 40 nM exendin 4 for 16, 24 and 48 hours.

FIG. 21. The effect of the multiple GLP-1/FDKP formulations on staurosporine-induced cell death. ARIP cells pre-treated with GLP-1 samples were exposed to 5 μM staurosporine for 4 hours and were analyzed with Cell Titer-Glo™ to determine cell viability. Samples were stressed at 4° and 40° C. for 4 weeks. Control samples, shown on the right (Media, GLP-1, STAU, GLP+STAU), illustrate the viability of cells in media (without GLP-1 or stauroporine), with GLP-1, with stauropsorine and with GLP-1 and staurosporine (note: the graph legend does not apply to the control samples). All of the results shown are averages of triplicate runs.

FIGS. 22A-22B. Pharmacokinetic studies depicting single intravenous injection (IV; FIG. 22A) and pulmonary insufflation (IS; FIG. 22B) in rats using various concentrations of GLP-1/FDKP formulations. The legends indicate the mass-to-mass percentage of GLP-1 to FDKP particles for the formulations analyzed.

FIGS. 23A-23B. Decrease in the cumulative food consumption in rats dosed with GLP-1/FDKP formulations at 2 hours (FIG. 23A) and 6 hours (FIG. 23B) post dose.

FIG. 24. Pharmacodynamic study of GLP-1/FDKP administered via pulmonary insufflation in male obese Zucker rats. The data depicts the glucose measurements at 0, 15, 30, 45, 60 and 90 minutes for the control (air; group 1) and the GLP-1/FDKP treated (group 2).

FIG. 25. Pharmacodynamic study of GLP-1/FDKP administered via pulmonary insufflation in male obese Zucker rats. The data depicts the GLP-1 measurements at 0, 15, 30, 45, 60 and 90 minutes for the control (air; group 1) and the GLP-1/FDKP treated (group 2).

FIG. 26. Pharmacodynamic study of GLP-1/FDKP administered via pulmonary insufflation in male obese Zucker rats. The data depicts the insulin measurements at 0, 15, 30, 45, 60 and 90 minutes for the control (air; group 1) and the GLP-1/FDKP treated (group 2).

FIG. 27. Pharmacokinetic study of GLP-1/FDKP with various GLP-1 concentrations administered via pulmonary insufflation in female rats. The data depicts the GLP-1 measurements at 0, 2, 5, 10, 20, 30, 40 and 60 minutes for the control (air; group 1) and GLP-1/FDKP treated groups 2, 3 and 4 administered 5%, 10% and 15% GLP-1 respectively.

FIG. 28. Pharmacokinetic study of GLP-1/FDKP with various GLP-1 concentrations administered via pulmonary insufflation in female rats. The data depicts the FDKP measurements at 0, 2, 5, 10, 20, 30, 40 and 60 minutes for the control (air; group 1) and GLP-1/FDKP treated groups 2, 3 and 4 administered 5%, 10% and 15% GLP-1 respectively.

FIG. 29. Pharmacodynamic study of GLP-1/FDKP in female rats administered GLP-1/FDKP containing 15% GLP-1 (0.3 mg GLP-1) via a single daily pulmonary insufflation (n=10) for 4 consecutive days. The data depicts average food consumption measured at predose, 1, 2, 4 and 6 hours post dose for 4 consecutive days.

FIG. 30. Pharmacodynamic study of GLP-1/FDKP in female rats administered GLP-1/FDKP containing 15% GLP-1 (0.3 mg GLP-1) via a single daily pulmonary insufflation (n=10) for 4 consecutive days. The data depicts average body weight measured at predose, 1, 2, 4 and 6 hours post dose for 4 consecutive days.

FIG. 31. Toxicokinetic study of GLP-1/FDKP in monkeys administered GLP-1/FDKP via oronasal administration once daily (for 30 minutes a day) for 5 consecutive days. The data depicts the peak plasma concentrations (Cₘₐₓ) of GLP-1 in males and females. Animals received control (air; group 1), 2 mg/kg FDKP (group 2) or 0.3, 1.0, or 2.0 mg/kg GLP-1/FDKP (groups 3, 4, and 5 respectively).

### DETAILED DESCRIPTION OF THE PREFERRED EMBODIMENT

Stable, inhalable glucagon-like peptide 1 (GLP-1) formulations for use as pharmaceutics are deficient in the art. This is due to the instability of GLP-1 peptide in vivo. GLP-1 compounds tend to remain in solution under a number of conditions, and have a relatively short in vivo half-life when administered as a solution formulation. Further, dipeptidyl-peptidase IV (DPP-IV), which is found to be present in various biological fluids such as the lung and blood, greatly reduces the biological half-life of GLP-1 molecules. For example, the biological half-life of GLP-1(7-37) has been shown to be 3 to 5 minutes; see U.S. Pat. No. 5,118,666. GLP-1 has also been shown to undergo rapid absorption in vivo following parenteral administration. Similarly, amide GLP-1(7-36) has a half-life of about 50 minutes when administered subcutaneously; see also U.S. Pat. No. 5,118,666.

The rapid clearance and short half-life of GLP-1 compositions in the art present a deficiency that the current invention overcomes. The present invention overcomes the deficiencies in the art by providing an optimized native GLP-1/FDKP (fumaryl diketopiperazine) formulation especially suited for pulmonary delivery. In other particular aspects, the present invention provides formulations of a native GLP-1 molecule that can elicit a GLP-1 response in vivo. Use of variants of native GLP-1 in such formulations is also contemplated.

To overcome the deficiencies in the art, the present invention provides formulations of GLP-1 in combination with diketopiperazine (DKP) particles. In particular embodiments of the invention, the GLP-1/DKP formulations are provided for administration to a subject. In further particular embodiments, the GLP-1/DKP formulations comprise fumaryl diketopiperazine (FDKP), but are not limited to such, and can include other DKPs (asymmetrical DKPs, xDKPs) such as 2,5-diketo-3,6-di(4-succinyl-aminobutyl)piperazine (SDKP), asymmetrical diketopiperazines including ones substituted at only one position on the DKP ring (for example “one armed” analogs of FDKP), and DKP salts. In other particular embodiments of the invention, administration of the GLP-1/FDKP formulation is by pulmonary delivery.

In developing therapeutic formulations of GLP-1 molecules the structural characteristics of GLP-1 in solution were evaluated by employing various biophysical and analytical techniques which included far-ultraviolet circular dichroism (far-UV CD), near-ultraviolet circular dichroism (near-UV CD), intrinsic fluorescence, fourier transform infrared spectroscopy (FTIR), high pressure liquid chromatography (HPLC), and mass spectroscopy (MS). The technique of circular dichroism (CD) is a powerful tool used to analyze the structural changes of a protein under varying experimental conditions and is well known in the art. The experimental conditions under which these analyses were conducted included: the effects of concentration, ionic strength, temperature, pH, oxidative stress, agitation, and multiple freeze-thaw cycles on the GLP-1 peptide. These analyses were designed to characterize the major routes of degradation as well as to establish conditions that manipulate the structure of GLP-1 peptide in order to achieve preferred GLP-1/DKP formulations having desirable pharmacokinetic (PK) and pharmacodynamic (PD) characteristics.

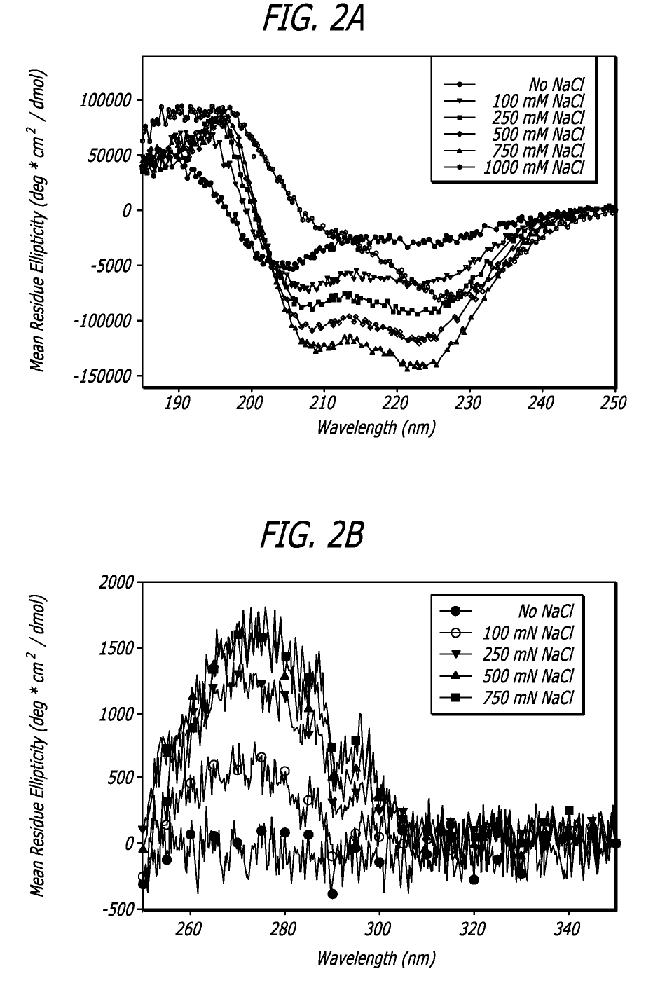

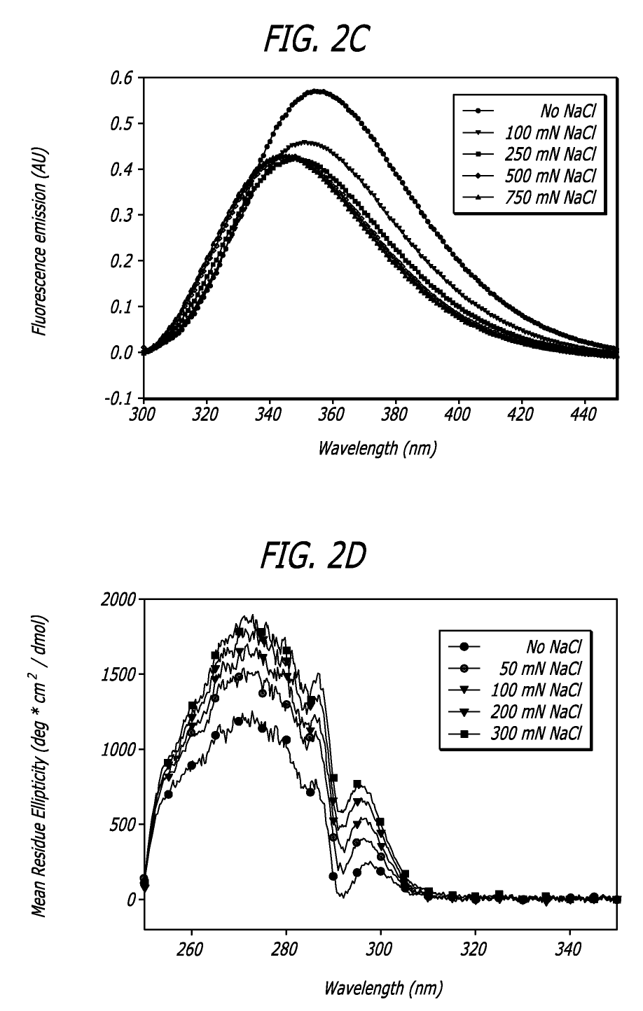

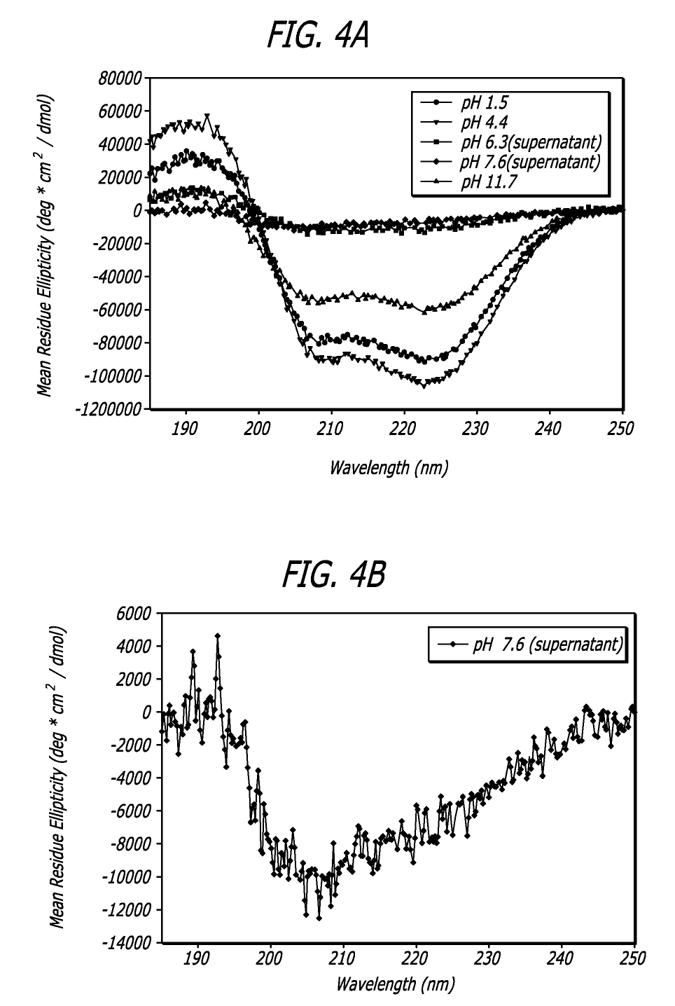

It was observed that as the concentration of GLP-1 increased, the secondary structure of the peptide was transformed from a predominantly unstructured conformation to a more helical conformation. Increasing the ionic strength in solution caused the structure of GLP-1 to increase until it reversibly precipitated. The presence of NaCl increased the tertiary structure of GLP-1 as is evident by an increase in intensity of the nearCD bands as depicted in FIG. 2D. This occurs even for the low concentrations of the peptide where there is no evidence of self-association. Increased ionic strength readily converted unstructured GLP-1 into the α-helical form as depicted by the farCD minima shifts toward 208 nm and 222 nm, (FIG. 2A) and self-associated conformations as depicted by the tryptophan emission shifts to lower wavelength with increased salt and the nearCD patterns in FIGS. 2B and 2D. Temperature and pH affected the conformations of GLP-1 differently in that the unordered structure of GLP-1 was not altered by either of these parameters. On the other hand, the self-associated conformation of GLP-1 was found to be sensitive to thermal denaturation and its solubility sensitive to pH as depicted in FIGS. 4A and 4B which shows GLP-1 peptide reversibly precipitates between pH 6.3-7.6 at a peptide concentration of 10 mg/ml. The various conformations of GLP-1 were found to be generally stable to agitation and multiple freeze-thaw cycles. Neither deamidation nor oxidation was observed for GLP-1.

Adsorption of GLP-1 to FDKP particles was also observed under a variety of conditions which included variation in pH, GLP-1 concentration, and in the concentration of various surfactants, salts, ions, chaotropes and lyotropes, stabilizers, and alcohols. The absorption of GLP-1 to FDKP particles was found to be affected strongly by pH, specifically, binding occurred at about pH 4.0 or greater. Other excipients were found to have a limited effect on the absorption of GLP-1 to FDKP particles.

In developing the GLP-1/DKP formulations of the present invention, a number of parameters that would affect or impact its deliverability and absorption in vivo were evaluated. Such parameters included, for example, the structure of the GLP-1 peptide, the surface charges on the molecule under certain formulation conditions, solubility and stability as a formulation, as well as susceptibility to serine protease degradation and in vivo stability; all of which play a critical role in generating a formulation that can be readily absorbed which exhibits an extended biological half-life.

The stability of GLP-1/FDKP formulations obtained was tested under a variety of conditions both in vitro and in vivo. The stability of GLP-1 was analyzed by HPLC analysis and cell-based assays. In addition, stability of GLP-1 was examined in lung lavage fluid (which contains DPP-IV). It was also found that the stability of native GLP-1 was concentration dependent in solution.

In vitro GLP-1 biological activity studies were also employed for studies of GLP-1/FDKP loading, and determining the effect in vivo. This strategy contributed to further identification of lead GLP-1/FDKP formulation methods. Further, based on the fact that GLP-1 has been shown to play a role in increasing 1-cells mass by inhibiting apoptosis, stimulating β-cell proliferation and islet neogenesis, the proliferative and anti-apoptotic potential of the GLP-1/FDKP formulations of the invention were examined through a cell-based assay.

Thus, the present invention provides optimized formulations comprising native human GLP-1 combined with fumaryl diketopiperazine (FDKP) that are stable and resistant to degradation.

### II. GLP-1 Molecules

In particular embodiments of the present invention there are provided optimized formulations comprising native human glucagon-like peptide 1 (GLP-1) combined with a diketopiperazine such as fumaryl diketopiperazine (FDKP). Such GLP-1/FDKP formulations of the present invention are stable and resistant to degradation.

Human GLP-1 is well known in the art and originates from the preproglucagon polypeptide synthesized in the L-cells in the distal ileum, in the pancreas and in the brain. GLP-1 is a 30-31 amino acid peptide that exists in two molecular forms, 7-36 and 7-37, with the 7-36 form being dominant. Processing of preproglucagon to GLP-1(7-36) amide and GLP-1(7-37) extended form occurs mainly in the L-cells. It has been shown in the art that, in the fasted state, plasma levels of GLP-1 are about 40 pg/ml. After a meal, GLP-1 plasma levels rapidly increase to about 50-165 pg/ml.

The term “GLP-1 molecules” as used herein refers to GLP-1 proteins, peptides, polypeptides, analogs, mimetics, derivatives, isoforms, fragments and the like. Such GLP-1 molecules may include naturally occurring GLP-1 polypeptides (GLP-1(7-37)OH, GLP-1(7-36)NH₂) and GLP-1 metabolites such as GLP-1(9-37). Thus, in particular embodiments of the invention, GLP-1 molecules include: a native GLP-1, a GLP-1 analog, a GLP-1 derivative, a dipeptidyl-peptidase-IV (DPP-IV) protected GLP-1, a GLP-1 mimetic, a GLP-1 peptide analog, or a biosynthetic GLP-1 analog.

As used herein, an “analog” includes compounds having structural similarity to another compound. For example, the anti-viral compound acyclovir is a nucleoside analogue and is structurally similar to the nucleoside guanosine which is derived from the base guanine. Thus, acyclovir mimics guanosine (is biologically analogous with) and interferes with DNA synthesis by replacing (or competing with) guanosine residues in the viral nucleic acid and prevents translation/transcription. Thus, compounds having structural similarity to another (a parent compound) that mimic the biological or chemical activity of the parent compound are analogs. There are no minimum or maximum numbers of elemental or functional group substitutions required to qualify a compound as an analog provided the analog is capable of mimicking, in some relevant fashion, either identically, complementarily or competitively, with the biological or chemical properties of the parent compound. Analogs can be, and often are, derivatives of the parent compound (see “derivative” infra). Analogs of the compounds disclosed herein may have equal, lesser or greater activity than their parent compounds.

As used herein, a “derivative” is a compound made from (or derived from), either naturally or synthetically, a parent compound. A derivative may be an analog (see “analog” supra) and thus may possess similar chemical or biological activity. However, unlike an analog, a derivative does not necessarily have to mimic the biological or chemical activity of the parent compound. There are no minimum or maximum numbers of elemental or functional group substitutions required to qualify a compound as a derivative. For example, while the antiviral compound ganclovir is a derivative of acyclovir, ganclovir has a different spectrum of anti-viral activity and different toxicological properties than acyclovir. Derivatives of the compounds disclosed herein may have equal, less, greater or even no similar activity when compared to their parent compounds.

As used herein, a “metabolite” is any intermediate or product of metabolism and includes both large and small molecules. As used herein and where appropriate, the definition applies to both primary and secondary metabolites. A primary metabolite is directly involved in normal growth, development, and reproduction of living organisms. A secondary metabolite is not directly involved in those processes, but typically has important ecological function (e.g., an antibiotic).

As used herein, the term “biosynthetic” refers to any production of a chemical compound by a living organism.

As used herein, “particle-forming” refers to chemical, biosynthetic, or biological entities or compounds that are capable of forming solid particles, usually in a liquid medium. The formation of particles typically occurs when a particle-forming entity is exposed to a certain condition(s) such as, for example, changes in pH, temperature, moisture, and/or osmolarity/osmolality. Exposure to the condition(s) may result in, for example, binding, coalescence, solidification and/or dehydration such that a particle is formed. A precipitation reaction is one example of a particle-forming event.

As used herein, “co-solution” is any medium comprised of at least two chemical, biological and/or biosynthetic entities. For example, a co-solution may be formed by combining a liquid comprising at least one chemical, biological and/or biosynthetic entity with a solid comprising a chemical, biological and/or biosynthetic entity. In another example, a co-solution may be formed by combining a liquid comprising at least one chemical, biological and/or biosynthetic entity with another liquid comprising a chemical, biological and/or biosynthetic entity. In a further example, a co-solution may be formed by adding at least two solids, each comprising at least one chemical, biological and/or biosynthetic entity, into a single solution.

Native GLP-1, as contemplated in the present invention, is a polypeptide having the amino acid sequence of SEQ ID NO. 1 or SEQ ID NO. 2. Native GLP-1 peptide undergoes rapid cleavage and inactivation within minutes in vivo.

GLP-1 analogs of the present invention may include the exendins, which are peptides found to be GLP-1 receptor agonists; such analogs may further include exendins 1 to 4. Exendins are found in the venom of the Gila-monster and share about 53% amino acid homology with mammalian GLP-1. Exendins also have similar binding affinity for the GLP-1 receptor. Exendin-3 and exendin-4 were reported to stimulate cAMP production in, and amylase release from, pancreatic acinar cells (Malhotra et al., 1992; Raufman et al., 1992; Singh et al., 1994). The use of exendin-3 and exendin-4 as insulinotrophic agents for the treatment of diabetes mellitus and the prevention of hyperglycemia has been proposed (U.S. Pat. No. 5,424,286).

Carboxyl terminal fragments of exendin such as exendin[9-39], a carboxyamidated molecule, and fragments 3-39 through 9-39 have been reported to be potent and selective antagonists of GLP-1 (Goke et al., 1993; Raufman et al., 1991; Schepp et al., 1994; Montrose-Rafizadeh et al., 1996). The literature has also demonstrated that exendin[9-39] blocks endogenous GLP-1 in vivo, resulting in reduced insulin secretion (Wang et al., 1995; D'Alessio et al., 1996). Exendin-4 potently binds to GLP-1 receptors on insulin-secreting β-TC1 cells, to dispersed acinar cells from pancreas, and to parietal cells from stomach. Exendin-4 peptide also plays a role in stimulating somatostatin release and inhibiting gastrin release in isolated stomachs (Goke et al., 1993; Schepp et al., 1994; Eissele et al., 1994). In cells transfected with the cloned GLP-1 receptor, exendin-4 is reportedly an agonist, i.e., it increases cAMP, while exendin[9-39] is identified as an antagonist, i.e., it blocks the stimulatory actions of exendin-4 and GLP-1. exendin has also been found to be resistant to degradation.

Another embodiment the present invention contemplates the use of peptide mimetics. Peptide mimetics, as are know to the skilled artisan, are peptides that biologically mimic active determinants on hormones, cytokines, enzyme substrates, viruses or other bio-molecules, and may antagonize, stimulate, or otherwise modulate the physiological activity of the natural ligands. Peptide mimetics are especially useful in drug development. See, for example, Johnson et al., “Peptide Turn Mimetics” in BIOTECHNOLOGY AND PHARMACY, Pezzuto et al., Eds., Chapman and Hall, New York (1993). The underlying rationale behind the use of peptide mimetics is that the peptide backbone of proteins exists chiefly to orient amino acid side chains in such a way as to facilitate molecular interactions. A peptide mimetic is expected to permit molecular interactions similar to the natural molecule.

In further embodiments it is contemplated that the GLP-1 molecules of the invention will have at least one biological activity of native GLP-1 such as the ability to bind to the GLP-1 receptor and initiate a signal transduction pathway resulting in insulinotropic activity. In further embodiments of the invention, a GLP-1 molecule may be a peptide, polypeptide, protein, analog, mimetic, derivative, isoform, fragment and the like, that retains at least one biological activity of a naturally-occurring GLP-1. GLP-1 molecules may also include the pharmaceutically acceptable salts and prodrugs, and salts of the prodrugs, polymorphs, hydrates, solvates, biologically-active fragments, biologically active variants and stereoisomers of the naturally-occurring human GLP-1 as well as agonist, mimetic, and antagonist variants of the naturally-occurring human GLP-1, the family of exendins including exendins 1 through 4, and polypeptide fusions thereof. A GLP-1 molecule of the invention may also include a dipeptidyl-peptidase-IV (DPP-IV) protected GLP-1 that prevents or inhibits the degradation of GLP-1.

GLP-1 molecules of the present invention include peptides, polypeptides, proteins and derivatives thereof that contain amino acid substitutions, improve solubility, confer resistance to oxidation, increase biological potency, or increase half-life in circulation. Thus, GLP-1 molecules as contemplated in the present invention comprise amino acid substitutions, deletions or additions wherein the amino acid is selected from those as are well known in the art. The N- or C-termini of the molecule may also be modified such as by acylation, acetylation, amidation, but is not limited to such. Thus, in the present invention, the term “amino acid” refers to naturally occurring and non-naturally occurring amino acids, as well as amino acid analogs and amino acid mimetics that function in a manner similar to naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analog refers to compounds that have the same basic chemical structure as a naturally occurring amino acid, i.e., an α carbon that is bound to a hydrogen, a carboxyl group, an amino group, and an R group, such as, homoserine, norleucine, norvaline, methionine sulfoxide, methionine methyl sulfonium, citrulline, hydroxyl glutamic acid, hydroxyproline, and praline. Such analogs have modified R groups (such as norleucine), but retain the same basic chemical structure as a naturally occurring amino acid. Amino acids contemplated in the present invention also include β-amino acids which are similar to α-amino acids in that they contain an amino terminus and a carboxyl terminus. However, in β-amino acids two carbon atoms separate these functional termini. β-amino acids, with a specific side chain, can exist as the R or S isomers at either the alpha (C2) carbon or the beta (C3) carbon. This results in a total of four possible diastereoisomers for any given side chain.

GLP-1 molecules of the present invention may also include hybrid GLP-1 proteins, fusion proteins, oligomers and multimers, homologues, glycosylation pattern variants, and muteins thereof, wherein the GL-P-1 molecule retains at least one biological activity of the native molecule, and further regardless of the method of synthesis or manufacture thereof including, but not limited to, recombinant (whether produced from cDNA, genomic DNA, synthetic DNA or other form of nucleic acid), synthetic, and gene activation methods. Recombinant DNA technology is well known to those of ordinary skill in the art (see Russell, D. W., et al., Molecular Cloning: A Laboratory Manual, Cold Spring Harbor, N.Y., 2001.

### III. Diketopiperazines

Diketopiperazines, are well known in the art for their ability to form microparticles that are useful for drug delivery and stabilization. In the present invention diketopiperazines are employed to facilitate the absorption of GLP-1 molecules thereby providing a stable formulation that is resistant to degradation.

Various methodologies may be employed wherein diketopiperazines can be formed into particles that incorporate GLP-1 molecules, or particles onto which GLP-1 molecules can be adsorbed. This may involve mixing of the diketopiperazine solutions with solutions or suspensions of GLP-1 molecules followed by precipitation and subsequent formation of particles comprising diketopiperazine and GLP-1. Alternatively, the diketopiperazine can be precipitated to form particles and subsequently mixed with a solution of GLP-1 molecules. Association between the diketopiperazine particle and the GLP-1 molecule can be driven by solvent removal or a specific step, such as a pH adjustment, can be included prior to drying in order to promote the association.

In a preferred embodiment, diketopiperazines of the present invention include but are not limited 3,6-di(fumaryl-4 aminobutyl)-2,5-diketopiperazine also known as (E)-3,6-bis[4-(N-carboxyl-2-propenyl)amidobutyl]-2,5-diketopiperazine (which may also be referred to as fumaryl diketopiperazine or FDKP).

Other diketopiperazines contemplated in the present invention include, without limitation, derivatives of 3,6-di(4-aminobutyl)-2,5-diketopiperazine such as: 3,6-di(succinyl-4-aminobutyl)-2,5-diketopiperazine (also referred to herein as 3,6-bis(4-carboxypropyl)amidobutyl-2,5-diketopiperazine; succinyl diketopiperazine or SDKP); 3,6-di(maleyl-4-aminobutyl)-2,5-diketopiperazine; 3,6-di(citraconyl-4-aminobutyl)-2-5-diketopiperazine; 3,6-di(glutaryl-4-aminobutyl)-2,5-diketopiperazine; 3,6-di(malonyl-4-aminobutyl)-2,5-diketopiperazine; 3,6-di(oxalyl-4-aminobutyl)-2,5-diketopiperazine and derivatives therefrom. In other embodiments, the present invention contemplates the use of diketopiperazine salts. Such salts may include, for example, any pharmaceutically acceptable salt such as the Na, K, Li, Mg, Ca, ammonium, or mono-, di- or tri-alkylammonium (as derived from triethylamine, butylamine, diethanolamine, triethanolamine, or pyridines, and the like) salts of diketopiperazine. The salt may be a mono-, di-, or mixed salt. Higher order salts are also contemplated for diketopiperazines in which the R groups contain more than one acid group. In other aspects of the invention, a basic form of the agent may be mixed with the diketopiperazine in order to form a drug salt of the diketopiperazine, such that the drug is the counter cation of the diketopiperazine. An example of a salt as contemplated herein, includes in a non-limiting manner FDKP diNa. Drug delivery using DKP salts is taught in U.S. patent application Ser. No. 11/210,710, incorporated herein by reference for all it contains regarding DKP salts.

As disclosed elsewhere herein, the present invention also employs novel asymmetrical analogs of FDKP, xDKPs such as: (E)-3-(4-(3,6-dioxopiperazin-2-yl)butylcarbamoyl)-acrylic acid; (E)-3-(3-(3,6-dioxopiperazin-2-yl)propyl-carbamoyl)acrylic acid; and (E)-3-(4-(5-isopropyl-3,6-dioxopiperazin-2-yl)-butylcarbamoyl)acrylic acid and disclosed in U.S. Provisional Patent Application entitled “Asymmetrical FDKP Analogs for Use as Drug Delivery Agents” filed on even date herewith and incorporated herein in its entirety (Atty Docket No. 51300-00041)

Diketopiperazines can be formed by cyclodimerization of amino acid ester derivatives, as described by Katchalski, et al., (J. Amer. Chem. Soc. 68:879-80; 1946), by cyclization of dipeptide ester derivatives, or by thermal dehydration of amino acid derivatives in high-boiling solvents, as described by Kopple, et al., (J. Org. Chem. 33:862-64; 1968), the teachings of which are incorporated herein.

Methods for synthesis and preparation of diketopiperazines are well known to one of ordinary skill in the art and are disclosed in U.S. Pat. Nos. 5,352,461; 5,503,852; 6,071,497; 6,331,318; 6,428,771 and U.S. Patent Application No. 20060040953. U.S. Pat. Nos. 6,444,226 and 6,652,885, describe preparing and providing microparticles of diketopiperazines in aqueous suspension to which a solution of active agent is added in order to bind the active agent to the particle. These patents further describes a method of removing a liquid medium by lyophilization to yield microparticles comprising an active agent, altering the solvent conditions of such suspension to promote binding of the active agent to the particle is taught in U.S. Patent Application Ser. No. 60/717,524 and 11/532,063 both entitled “Method of Drug Formulation Based on Increasing the Affinity of Active Agents for Crystalline Microparticle Surfaces”; and Ser. No. 11/532,065 entitled “Method of Drug Formulation Based on Increasing the Affinity of Active Agents for Crystalline Microparticle Surfaces.” See also U.S. Pat. No. 6,440,463 and U.S. patent application Ser. No. 11/210,709 filed on Aug. 23, 2005 and U.S. patent application Ser. No. 11/208,087). In some instances, it is contemplated that the loaded diketopiperazine particles of the present invention are dried by a method of spraying drying as disclosed in, for example, U.S. patent application Ser. No. 11/678,046 filed on Feb. 22, 2006 and entitled “A Method For Improving the Pharmaceutic Properties of Microparticles Comprising Diketopiperazine and an Active Agent.” Each of these patents and patent applications is incorporated by reference herein for all they contain regarding diketopiperazines.

### IV. Therapeutic Formulations of GLP-1/DKP Particles

The present invention further provides a GLP-1/FDKP formulation for administration to a subject in need of treatment. A subject as contemplated in the present invention may be a household pet or human. In certain embodiments, the treatment is for Type II diabetes, obesity, cancer or any related diseases and/or conditions therefrom. Humans are particularly preferred subjects.

Other diseases or conditions contemplated in the present invention include, but are not limited to, irritable bowel syndrome, myocardial infarction, ischemia, reperfused tissue injury, dyslipidemia, diabetic cardiomyopathy, acute coronary syndrome, metabolic syndrome, catabolic changes after surgery, neurodegenerative disorders, memory and learning disorders, islet cell transplant and regenerative therapy or stroke. Other diseases and/or conditions contemplated in the present invention are inclusive of any disease and/or condition related to those listed above that may be treated by administering a GLP-1/FDKP dry powder formulation to a subject in need thereof. The GLP-1/FDKP dry powder formulation of the present invention may also be employed in the treatment of induction of beta cell differentiation in human cells of type-II diabetes and hyperglycemia.

In still a further embodiment of the present invention, it is contemplated that the subject may be a household pet or animal, including rats, rabbits, hamsters, guinea pigs, gerbils, woodchucks, cats, dogs, sheep, goats, pigs, cows, horses, monkeys and apes (including chimpanzees, gibbons, and baboons).

It is further contemplated that the GLP-1/FDKP particle formulations of the invention can be administered by various routes of administration known to persons of ordinary skill in the art and for clinical or non-clinical purposes. The GLP-1/FDKP compositions of the invention may be administered to any targeted biological membrane, preferably a mucosal membrane of a subject. Administration can be by any route, including but not limited to oral, nasal, buccal, systemic intravenous injection, subcutaneous, regional administration via blood or lymph supply, directly to an affected site or even by topical means. In preferred embodiments of the present invention, administration of GLP-1/FDKP composition is by pulmonary delivery.

Other alternative routes of administration that may be employed in the present invention may include: intradermal, intraarterial, intraperitoneal, intralesional, intracranial, intraarticular, intraprostatic, intrapleural, intratracheal, intravitreal, intravaginal, rectal, intratumoral, intramuscular, intravesicular, mucosally, intrapericardial, bronchial administration local, using aerosol, injection, infusion, continuous infusion, localized perfusion bathing target cells directly, via a catheter, via a lavage, in cremes, in lipid compositions (e.g., liposomes), or by other method or any combination of the foregoing as would be known to one of ordinary skill in the art (see, for example, Remington's Pharmaceutical Sciences, 1990, incorporated herein by reference for all it contains regarding methods of administration).

As a dry powder formulation, the GLP-1/DKP particles of the present invention can be delivered by inhalation to specific areas of the respiratory system, depending on the particle size. Additionally, the GLP-1/DKP particles can be made small enough for incorporation into an intravenous suspension dosage form. For oral delivery, the particles can be incorporated into a suspension, tablets or capsules. The GLP-1/DKP composition may be delivered from an inhalation device, such as a nebulizer, a metered-dose inhaler, a dry powder inhaler, and a sprayer.

In further embodiments, administration of an “effective amount” of a GLP-1/DKP formulation to a patient in need thereof is contemplated. An “effective amount” of a GLP-1/DKP dry powder formulation as contemplated in the present invention refers to that amount of the GLP-1 compound, analog or peptide mimetic or the like, which will relieve to some extent one or more of the symptoms of the disease, condition or disorder being treated. In one embodiment, an “effective amount” of a GLP-1/DKP dry powder formulation would be that amount of the GLP-1 molecule for treating diabetes by increasing plasma insulin levels, reducing or lowering fasting blood glucose levels, and increasing pancreatic beta cell mass by at least about 1, 2, 3, 4, 5, 6, 7, 8, 9, 10, 15, 20, 25, 30, 35, 40, 45, 50%, or greater, but not limited to such. In another preferred embodiment the present invention contemplates treating obesity by administering to a subject in need of such treatment a pharmaceutically effective amount of the GLP-1 molecule. In such instances an “effective amount” of a GLP-1/DKP dry powder formulation would be that amount of the GLP-1 molecule for treating obesity by reducing or lowering body weight by at least about 5, 10, 15, 20, 25, 30, 35, 40, 45, 50%, or greater, but is not limited to such. The present invention also contemplates administering an “effective amount” of a GLP-1/DKP dry powder formulation for controlling satiety, by administering to a subject in need of such treatment a pharmaceutically effective amount of the GLP-1 molecule. In a non-limiting manner, the GLP-1 molecule can be an exendin molecule such as exendin-1 or -4. In such instances, an “effective amount” of a GLP-1/DKP dry powder formulation would be that amount of the GLP-1 molecule that reduces the perception of hunger and food intake (as measured by mass or caloric content, for example) by at least about 5, 6, 7, 8, 9, 10, 11, 12, 13, 14, 15, 16, 17, 18, 19, 20, 21, 22, 23, 24, 25, 26, 27, 28, 29, 30, 35, 40, 45, 50%, or greater, but not limited to such. An “effective amount” of a GLP-1/DKP dry powder formulation may be further defined as that amount sufficient to detectably and repeatedly ameliorate, reduce, minimize or limit the extent of the disease or condition or symptoms thereof. Elimination, eradication or cure of the disease or condition may also be possible utilizing an “effective amount” of the inventive formulation.

In administering a GLP-1/FDKP composition of the present invention to a subject in need thereof, the actual dosage amount of the composition can be determined on the basis of physical and physiological factors such as body weight, severity of condition, the type of disease being treated, previous or concurrent therapeutic interventions, idiopathy of the patient and the route of administration. A skilled artisan would be able to determine actual dosages based on one or more of these factors.

The GLP-1/DKP formulation of the present invention can be administered once or more than once, depending the disease or condition to be treated. Administration of the GLP-1/DKP formulation can be provided to the subject at intervals ranging over minutes, hours, days, weeks or months. In some instances, timing of the therapeutic regimen may be related to the half-life of the GLP-1 molecule upon administration. In further embodiments, in treating particular or complex diseases or conditions such as cancer, for example, it may be desirable to administer a GLP-1/DKP formulation of the present invention with a pharmaceutical excipient or agent. In such cases, an administration regimen may be dictated by the pharmaceutical excipient or agent.

### V. Examples

The following examples are included to demonstrate certain embodiments of the invention. It should be appreciated by those of skill in the art that the techniques disclosed in the examples, which elucidate representative techniques that function well in the practice of the present invention. However, those of skill in the art should, in light of the present disclosure, appreciate that many changes can be made in the specific embodiments that are disclosed and still obtain a like or similar result without departing from the spirit and scope of the invention

### Example 1

### Biophysical and Analytical Analyses of the Structure of GLP-1

To analyze both the structure and behavior of GLP-1 a number of biophysical and analytical techniques were employed. These techniques included far-ultraviolet circular dichroism (far-UV CD), near-ultraviolet circular dichroism (near-UV CD), intrinsic fluorescence, fourier transform infrared spectroscopy (FTIR), high pressure liquid chromatography (HPLC), and mass spectroscopy (MS); all of which are well know to one of ordinary skill in the art. A wide range of conditions were employed to investigate the effects of concentration, ionic strength, temperature, pH, oxidative stress, agitation, and multiple freeze-thaw cycles on the GLP-1 peptide; all of which are described in further detail below. These analyses were also employed to characterize the major routes of degradation and to establish conditions that manipulate peptide structure of GLP-1 in order to achieve certain GLP-1/DKP formulations.

Experimental Procedure

GLP-1 was purchased either from American Peptide (Sunnyvale, Calif.) or AnaSpec (San Jose, Calif.), or prepared in house (MannKind Corporation, Valencia, Calif.). Aqueous GLP-1 samples, of varying concentration, were analyzed at pH 4.0 and 20° C. (unless otherwise noted). Samples were generally prepared fresh and were mixed with the appropriate additive (e.g., salt, pH buffer, H₂O₂ etc., if any), prior to each experiment. Secondary structural measurements of GLP-1 under various conditions were collected with far-UV CD and transmission fourier transform infrared spectroscopy (FTIR). In addition, both near-UV CD and intrinsic fluorescence were employed to analyze the tertiary structure of GLP-1 by monitoring the environments surrounding its aromatic residues, namely tryptophan.

Concentration-Dependent Structures of GLP-1

Circular dichroism (CD) spectra was used to analyze the α-helix, random coil, β-pleated sheet, 1-turns and random coil that may be displayed by a molecule such as a protein or peptide. In particular, far-UV CD was used to determine the type of secondary structure, for example pure α-helix, β-sheet, etc., in proteins and peptides. On the other hand, near-UV CD was used to analyze the tertiary structures of a molecule. Thus, in order to examine the effect of concentration on GLP-1 structures, far- and near-UV CD techniques were employed.

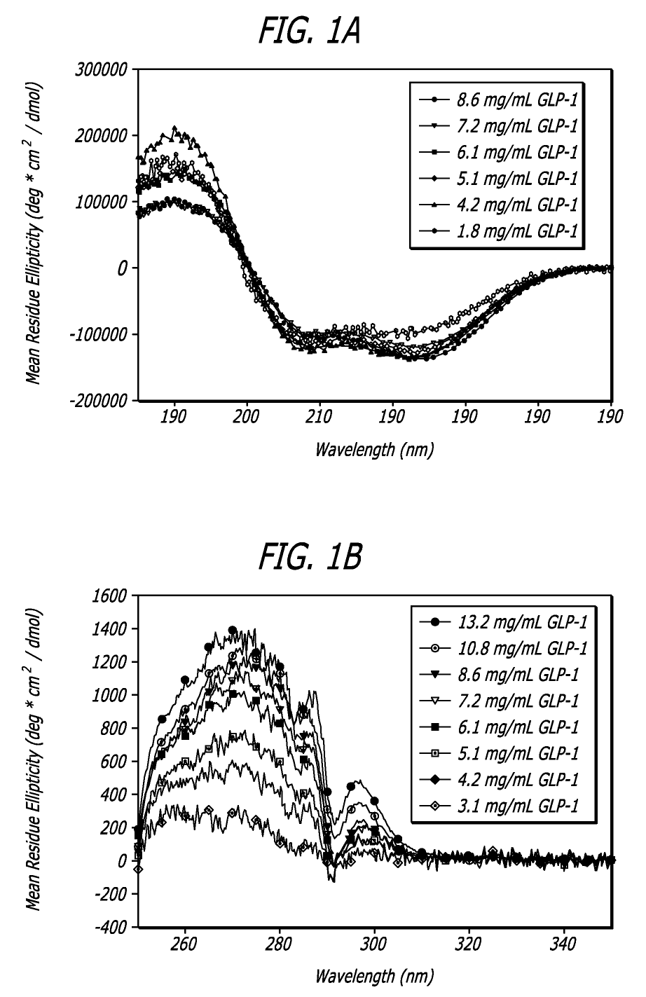

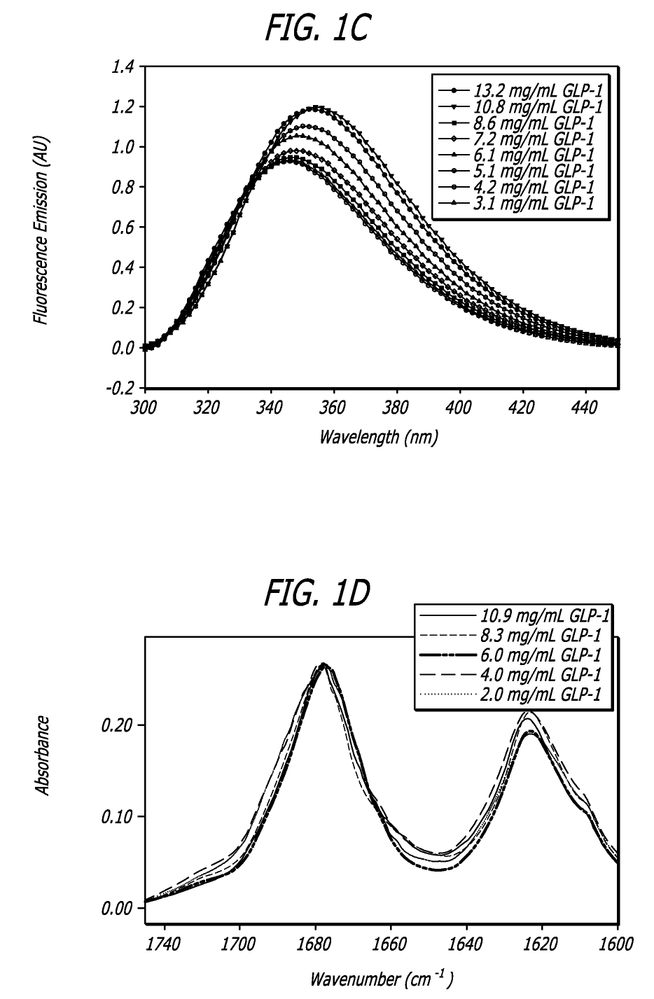

The far-UV CD in FIG. 1A demonstrated that GLP-1 forms two distinct structures which include α-helices and random coils, over a wide range of concentrations (for example: 1.8, 4.2, 5.1, 6.1, 7.2 and 8.6 mg/mL). At low concentrations (≦2 mg/mL), GLP-1 is primarily unstructured, as determined by the large single minima at 205 nm. As the concentration is increased, the peptide adopts an α-helical structure as determined by the two minima at 208 nm and 224 nm (FIG. 1A).

Tertiary structural analysis suggests that the high concentration structures of GLP-1 are self-associated conformations (i.e., oligomers). Both the near-UV CD and fluorescence emission data support this hypothesis. The positive bands between 250-300 nm in the near-UV CD (FIG. 1B) reveal that GLP-1 has a defined tertiary structure which increases at higher concentrations. More specifically, these bands indicate that the aromatic residues of the peptide are largely immobilized and exist in a well-defined environment.

Similarly, the fluorescence emission of GLP-1 at various concentrations (pH 4.0, 20° C.) showed that the aromatic residue tryptophan (which also displayed intense bands in near-UV CD spectra) exists in a well-defined tertiary structure; the data shown resulted from tryptophan excitation at 280 nm (FIG. 1C). The fluorescence maximum at 355 nm for low concentrations of GLP-1 indicated that the tryptophan is solvent exposed and that there is no significant tertiary structure. At high peptide concentrations, the maxima decreased in intensity and shifted to a lower wavelength, indicative of a more-defined tertiary structure.

In order to further determine the underlying secondary structure of GLP-1 self-associated conformation, FTIR analysis was performed at various concentrations (pH 4.0, 20° C.). The amide I band at 1656 cm⁻¹ clearly indicates that GLP-1 has a α-helical structure at concentrations ≧2 mg/mL (FIG. 1D). Therefore, GLP-1 does not form β-sheet structures; instead it is more likely that the peptide generates a helix bundle at high concentrations.

Additionally, it was experimentally shown that these various structures of GLP-1 were not generated via sample handling. Dilutions from a concentrated stock solution compared to GLP-1 prepared by directly dissolving the peptide in buffer generated similar far-UV CD, near-UV CD, and fluorescence emission spectra.

The Effect of Ionic Strength on GLP-1

Studies were also conducted to determine the effect of ionic strength on GLP-1 peptide. FIG. 2A (far-UV-CD) illustrates that increasing the concentration of salt (from 100 mM to 1000 mM) converts the unordered structure of GLP-1 into a α-helical conformation, as revealed by the minimas at 208 and 224 nm. Upon raising the NaCl concentration to 1M, much of the peptide (at 1.0 mg/mL) precipitates out of solution (FIG. 2A). Nevertheless, this type of precipitate was shown to dissolve upon dilution with water, thus establishing that at high ionic strength GLP-1 can be reversibly precipitated.

Salt was also shown to generate and improve the tertiary structure of GLP-1. This is exemplified in FIG. 2B (near-UV-CD) where 1.0 mg/mL GLP-1 displays no signal in the absence of salt, but exhibits a clear tertiary structure that intensifies with increasing ionic strength. These results were confirmed with the fluorescence emission of 1.0 mg/mL GLP-1 (FIG. 2C) at varying NaCl concentrations (pH 4.0, 20° C.) following tryptophan excitation at 280 nm. Increasing ionic strength caused the fluorescence maximum to shift to lower wavelengths, indicating that the tertiary structure of 1.0 mg/mL GLP-1 is both generated and enhanced.

Additionally, tertiary structural analysis of 10 mg/mL GLP-1 at varying ionic strength (pH 4.0, 20° C.) using near-UV CD spectra, demonstrated that the GLP-1 self-associated conformation is also enhanced with increased ionic strength (FIG. 2D).

The data therefore suggests that ionic strength has a dramatic effect on the structure of GLP-1, causing the protein both to assume an α-helical conformation and associate into oligomers. Further, increasing the ionic strength in solution causes the oligomerization of GLP-1 to increase until it reversibly precipitates. This occurrence is evident at low concentrations of the peptide, where there is initially no tertiary structure, as well with high concentrations of the peptide that already display substantial secondary and tertiary structure. Thus, increased ionic strength readily converts unstructured GLP-1 into the α-helical and self-associated conformations. Moreover, the observed spectroscopic results are comparable to the affects of increased peptide concentration shown previously.

The Effect of Temperature and pH on GLP-1

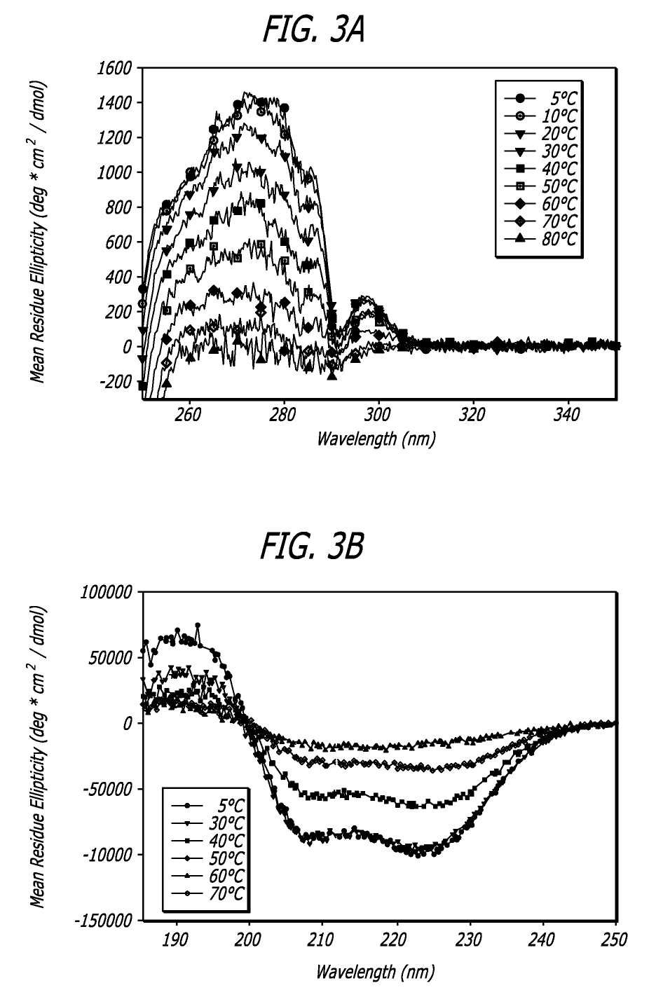

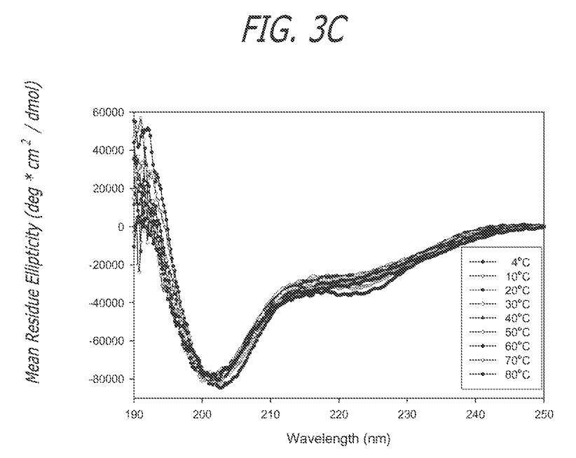

Studies were also conducted to determine whether the self-associated conformation of GLP-1 is sensitive to changes in either temperature or pH. FIG. 3A (near-UV-CD) demonstrates that the tertiary structure of 10 mg/mL GLP-1 significantly dissociates as temperature increases. On the other hand, temperature does not have an affect on low concentrations (0.05 mg/mL) of GLP-1 at various temperatures and pH 4.0; see FIGS. 3B and 3C (far-UV-CD). The far-UV CD illustrates that the peptide is insensitive to temperature. Therefore, increased molecular motion significantly hinders self-association of GLP-1.

Conversely, FIG. 4A (far-UV-CD) demonstrates that the solubility of the α-helical GLP-1 conformation is pH sensitive. Although the structure of 10 mg/mL GLP-1 is relatively uniform (i.e., GLP-1 remains helical) at pH 4.4 and below, some precipitation occurs when the pH is raised to near or at neutral (between pH 6.3 and 7.6) and an unordered spectrum is generated. Samples where precipitation occurred have less intensity as a result of less soluble GLP-1 being present in the solution. This unordered structure is determined by the single minima observed at 208 nm in FIG. 4A (far-UV-CD), which is further depicted in FIG. 4B (near-UV-CD) and likely results from a decrease of GLP-1 in solution following precipitation. This precipitation may occur when the pH is raised above the pI of 5.5 for GLP-1. However, as the pH was raised from near neutral to 11.7, most of the precipitate re-dissolved, indicating the precipitation is reversible. The remaining un-dissolved precipitate for GLP-1 at pH 11.7 would cause the amount of peptide in solution to decrease and hence reduce the intensity of the far-UV CD spectrum, as observed in FIG. 4A. It was also observed that GLP-1 is extremely insoluble when lyophilized GLP-1 powder is mixed with pH 9 buffer to a high concentration of GLP-1.

Stability of GLP-1

The stability of GLP-1 peptide was examined by determining its resistance to deamidation and oxidation in addition to the effects of agitation and freeze-thaw cycles.

GLP-1 (1 mg/mL) at pH 10.5, was incubated for 5 days at 40° C. following which reverse-phase HPLC and electrospray mass spectrometry (MS) were performed for deamidation and oxidation analyses. Oxidation studies were also conducted on GLP-1 samples (1 mg/mL) incubated for 2 hours in the presence of 0.1% H₂O₂ using both HPLC and MS.

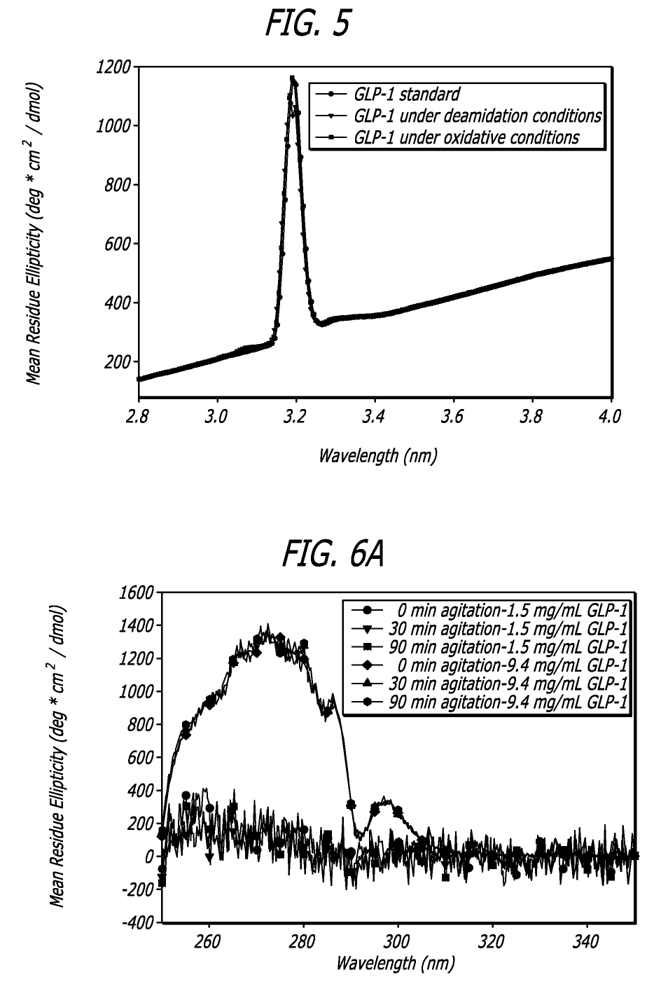

FIG. 5 depicts the stability of GLP-1 under conditions of deamidation and oxidation. The HPLC chromatograms illustrate that GLP-1 elutes at the same retention time and that no degradation peaks result for the destabilizing conditions analyzed. Additionally, MS analyses yielded a similar mass for all the samples, 3297 g/mol, indicating that the mass is unaltered. The data also illustrates that the peptide remains pure and intact when incubated under various conditions. Thus, deamidation of GLP-1 was not observed. GLP-1 was also shown to be stable to oxidative stress as observed in the presence of 0.1% H₂O₂, where the purity and mass of GLP-1 remained intact, as determined by HPLC and MS respectively. Overall, there were no changes in the retention times or the mass values and no degradation peaks resulted, thereby demonstrating that GLP-1 peptide is resistance to both deamidation and oxidation.

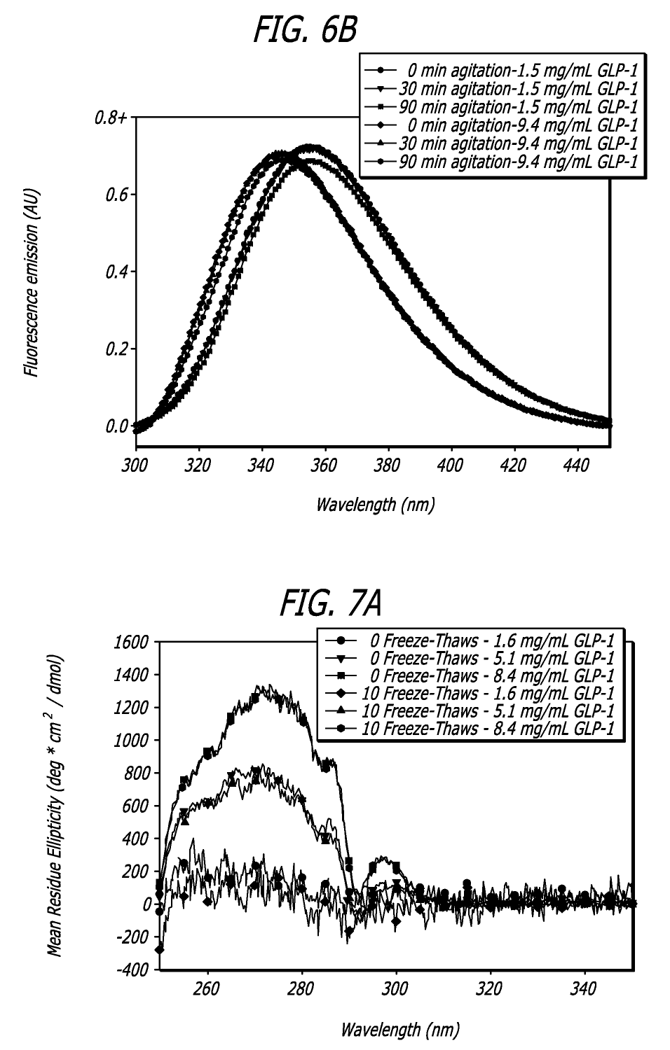

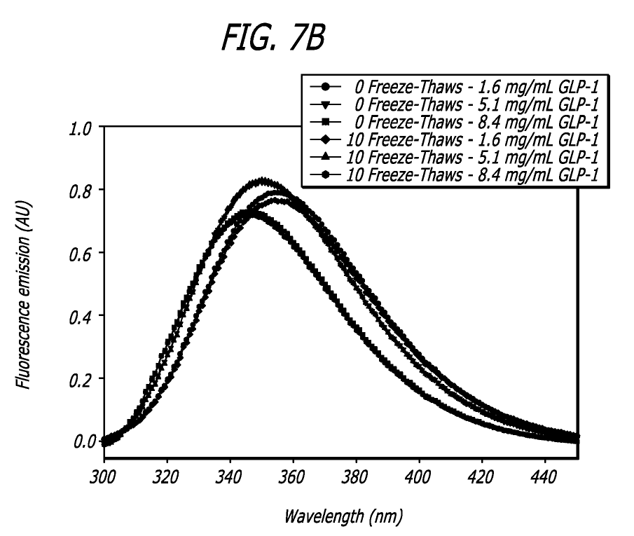

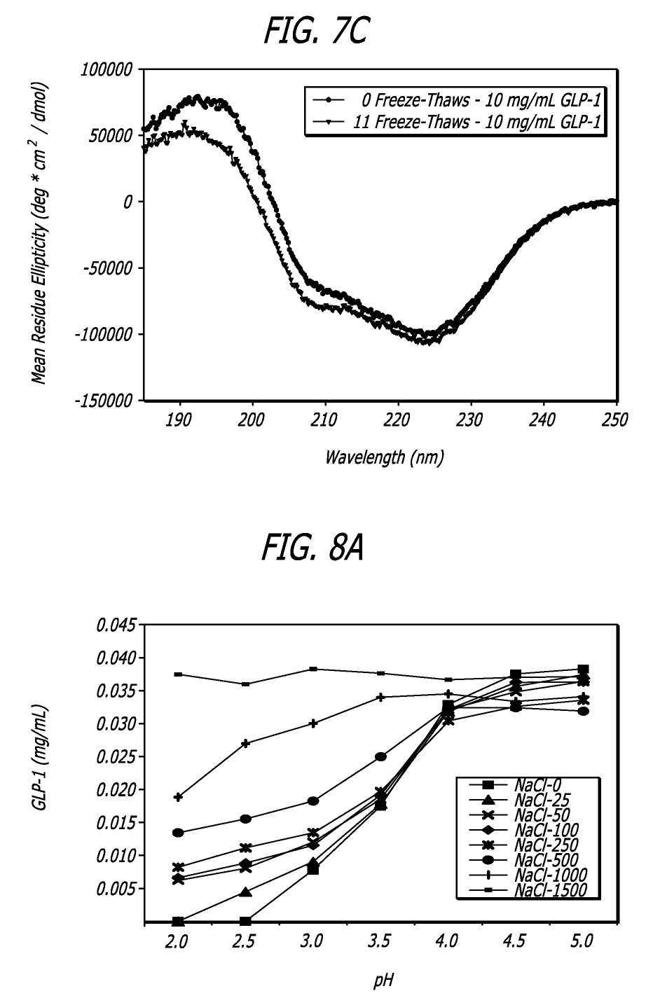

The effects of agitation and consecutive freeze-thaw cycles on various concentrations of GLP-1 were analyzed with near-UV CD, and intrinsic fluorescence. Agitation of 9.4 and 1.5 mg/mL GLP-1 produced no significant alterations in the peptide as observed by near-UV CD (FIG. 6A), and fluorescence emission (FIG. 6B). The samples were agitated for 30 and 90 min at room temperature and the fluorescence emission spectra were collected after tryptophan excitation at 280 nm. In independent freeze-thaw studies, solutions containing GLP-1 (pH 4.0) at 1.6, 5.1 and 8.4 mg/mL were frozen at −20° C. and thawed at room temperature. The effect of 10 freeze-thaw cycles on GLP-1 was conducted and analyzed by near-UV CD (FIG. 7A) and fluorescence emission (FIG. 7B). Fluorescence emission spectra were collected after tryptophan excitation at 280 nm. Both analyses show that the tertiary structure of the peptide does not notably change due to multiple freeze-thaw cycles. In similar experiments, the effect of 11 freeze-thaw cycles on the secondary structure of 10 mg/mL GLP-1 (pH 4.0) was analyzed (FIG. 7C). The far-UV CD illustrates that the secondary structure of the peptide does not change significantly as a result of multiple freeze-thaw cycles.

Overall, the biophysical analyses obtained from the above experiments showed that the structure of the GLP-1 peptide is strongly influenced by its concentration in solution. As the concentration of GLP-1 was increased, α-helical structures became more prominent. In addition, increasing the ionic strength enhanced, and in some cases generated, tertiary GLP-1 structures.

### Example 2

### GLP-1/FDKP Adsorption Studies

The interaction of GLP-1 with diketopiperazine (DKP) particles in suspension was evaluated by conducting adsorption studies. The variables in adsorption studies explored the effects of electrostatics, hydrogen bonding, water structure, protein flexibility, and specific salt-pairing interactions on the GLP-1/DKP interaction. In addition, several common protein stabilizers were tested for interference with GLP-1 adsorption to DKP surfaces.

Using pre-formed DKP suspension particles (i.e., FDKP), conditions where GLP-1 adsorbs to the surfaces of preformed DKP particles were studied. A FDKP particle suspension, in which the FDKP particles are pre-formed, was combined with 3× pH buffer and 3× solution of an additive or excipient. The final solution contained a FDKP concentration of 5, mg/ml and a GLP-1 concentration of 0.25, mg/ml (5% w/w). Unbound GLP-1 in the supernatant was filtered off the suspension. The FDKP particles with the associated GLP-1 protein were dissolved (reconstituted) with 100, mM ammonium bicarbonate and filtered to separate out any aggregated GLP-1 protein. The amount of GLP-1 in both the supernatant and reconstituted fractions was quantitated by HPLC. A series of experiments were conducted in which conditions employed included use of additives such as salts, surfactants, ions, osmolytes, chaotropes, organics, and various concentrations of GLP-1. The results from these studies are described below.

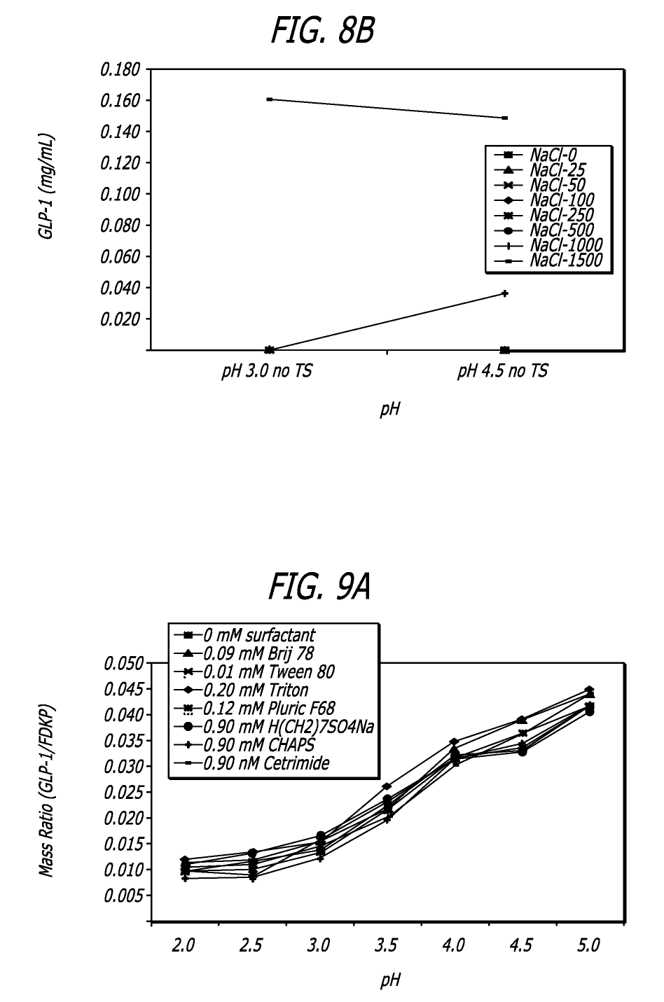

Salt studies.—The effect of salt on the binding of GLP-1 to FDKP particles was observed by HPLC analysis. Loading of the GLP-1/FDKP particles was performed at 5 mg/mL FDKP and 0.25 mg/mL GLP-1 in the presence of 0, 25, 50, 100, 250, 500, 1000 and 1500 mM NaCl (FIG. 8A). The amount of GLP-1 detected in reconstituted FDKP-free control samples as a function of pH and NaCl concentration was also assessed (FIG. 8B). The pH in both data sets was controlled with a 20 mM phosphate/20 mM acetate mixture.

As observed in FIG. 8A, the optimal binding (adsorption) of GLP-1 to FDKP particles was strongly influenced by the pH of the suspension. At a pH of 4 and above, about 3.2% to about 4% binding of GLP-1 to FDKP particles was observed where the GLP-1/FDKP ratio in solution was 5% w/w. Essentially no adsorption of GLP-1 to FDKP particles was evident at pH 2.0 in the presence of 0 and 25 mM NaCl, but some apparent loading was observed with increased ionic strength. GLP-1 precipitation was observed in the FDKP-free controls with ≧1M NaCl FIG. 8B. This apparent loading at ≧1M NaCl is the result of reversible precipitation (salting out) of the GLP-1 peptide at high ionic strength. High-salt controls of GLP-1 free of FDKP particles also exhibited high GLP-1 levels in the reconstituted samples, indicating that GLP-1 had been trapped in the filters when the supernatant had been collected. Below 1M NaCl, there was no evidence of GLP-1 precipitation in the absence of FDKP particles.

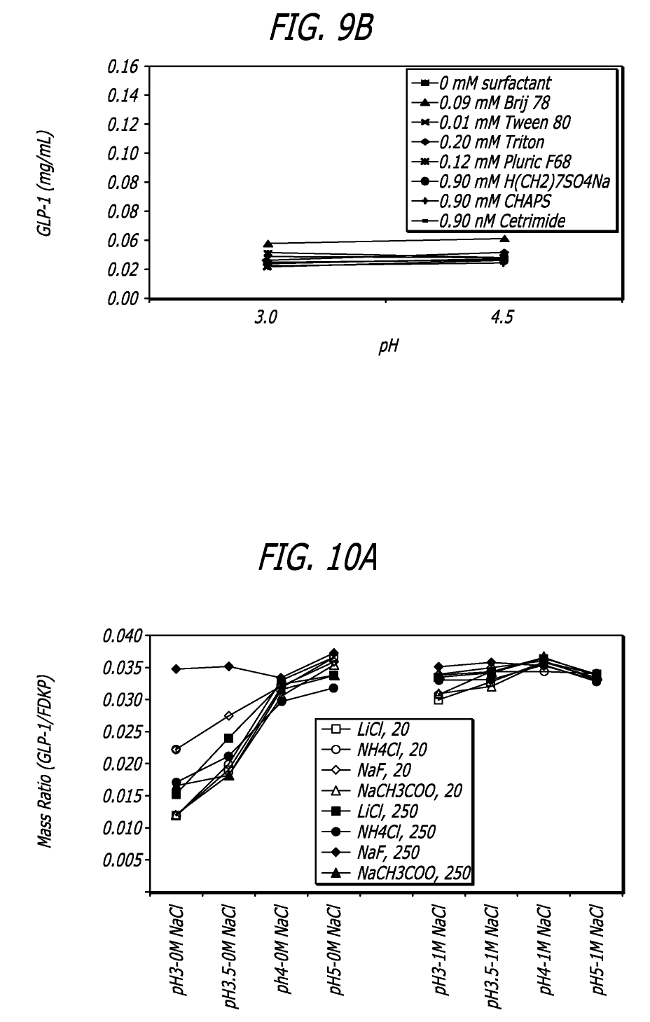

Surfactant studies.—The effect of surfactants on the binding of GLP-1 to FDKP particles was observed by HPLC analysis. Loading was performed at 5 mg/ml FDKP and 0.25 mg/mL GLP-1 in the presence of a surfactant (FIG. 9A). The amount of GLP-1 detected in reconstituted FDKP-free control samples as a function of pH and surfactant concentration was also assessed (FIG. 9B). The pH and the control sample conditions were as described for the above ionic strength study. Surfactants employed in this study included: Brij 78 at 0.09 mM, Tween 80 at 0.01 mM, Triton X at 0.2 mM, Pluronic F68 at 0.12 mM, H(CH₂)₇SO₄Na at 0.9 mM, CHAPS at 0.9 mM, Cetrimide at 0.9 mM. Loading curves for GLP-1 in the presence of each surfactant are shown are for GLP-1/FDKP as a function of pH.

The data show that the pH-adsorption curves for GLP-1/FDKP particles were not influenced by the presence of surfactants near their critical micelle concentration (CMC)— that is, the small range of concentrations separating the limit below which virtually no aggregates/micelles are detected and the limit above which virtually all additional surfactant molecules form aggregates. Therefore, it is further suggested that any of these surfactants could be used to optimize stability and/or pharmacokinetics (PK) as discussed below. As demonstrated above for the salt study, interaction of GLP-1 with FDKP particles was influenced by the pH of the suspension.

Ion studies. For this experiment, two different ion studies were run to determine the effect of ions on the binding of GLP-1 to FDKP particles. In both studies, Cl⁻ was the counterion for cations and Na⁺ was the counterion for anions. Loading of the GLP-1/FDKP particles was performed as described for the previous experiments. The pH was controlled as described supra. The samples were prepared with a pH buffer of either pH 3.0, 3.5, 4.0, or 5.0 in the presence and absence of NaCl (which was used to better assess cases of high ionic strength). Additional ions were included in individual samples as follows: LiCl at 20 or 250 mM, NH₄Cl at 20 or 250 mM, NaF at 20 or 250 mM, and NaCH₃COO at 20 or 250 mM.

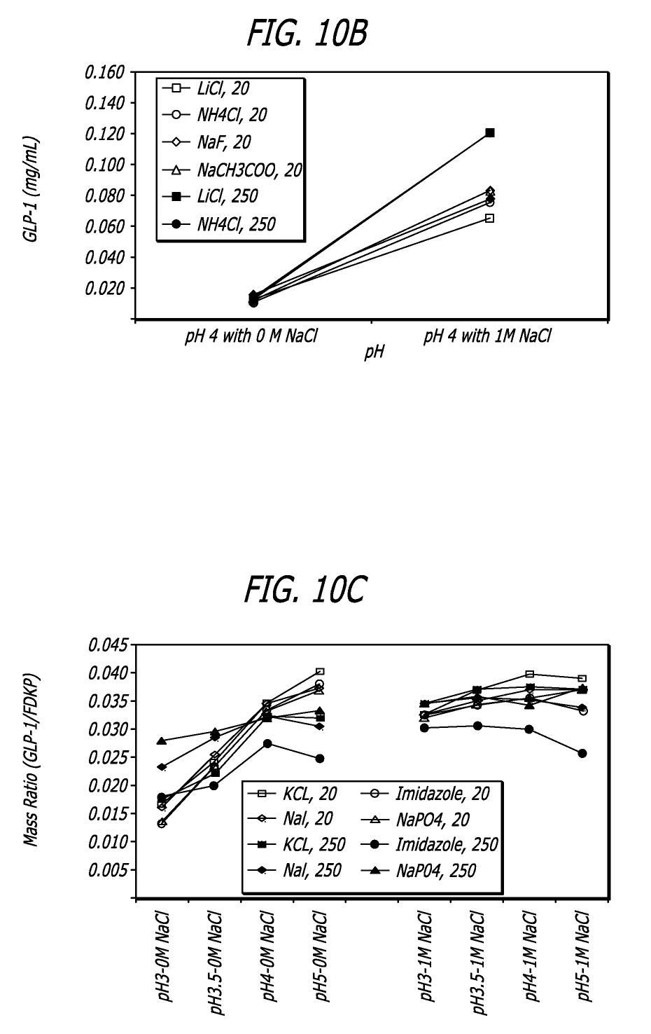

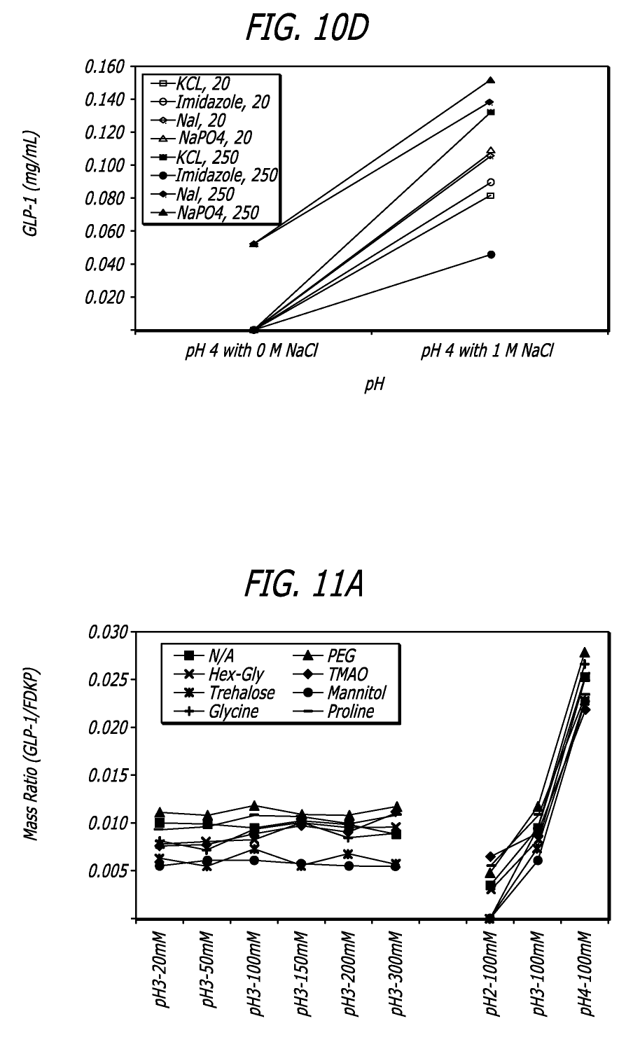

The data from the first ion study, as depicted in FIG. 10A, shows the loading curves for GLP-1/FDKP, as a function of pH and ions. In the absence of NaCl, fluoride at a concentration of either 20 or 250 mM strongly influenced (enhanced) adsorption at low pH with the NaF at a concentration of 250 mM exhibiting maximal binding regardless of pH. This pattern was observed due to the fluoride in the solution, not the sodium, because sodium bicarbonate did not have the same effects at 20 and 250 mM. Furthermore these effects were not a result of the sodium in the sample because salt at similar concentration, as shown in FIG. 8, did not show this effect. In the presence of 1M NaCl, all of the ions gave a high ‘apparent’ load. The ‘apparent’ load for the 1M NaCl samples resulted from the GLP-1 peptide salting out of solution in the presence of high ionic strength. This is further illustrated in FIG. 10B, which shows that GLP-1 is present in the reconstituted FDKP-free control samples containing 1M NaCl. The amount of GLP-1 detected for these control samples increased for larger ion concentrations, because they added to the total ionic strength in the samples.

In the second ion experiment (FIG. 10C) the GLP-1/FDKP samples were prepared in the presence of KCl at 20 or 250 mM, imidiazole at 20 or 250 mM, NaI at 20 or 250 mM, or NaPO₄ at 20 or 250 mM. The data shows that at 250 mM imidazole decreased loading in the presence of 1M NaCl and both 250 mM phosphate and 250 mM gave a high ‘apparent’ load (FIG. 10C). Based on the amount of GLP-1 detected in the reconstituted FDKP-free control samples at 0M and 1M NaCl concentrations (FIG. 10D), these affects resulted from the influence of the ions on the GLP-1 peptide itself and not on the interaction of the peptide with FDKP particles. Sodium phosphate and sodium iodide caused some salting-out of GLP-1 in the absence of NaCl. Additionally, imidazole helped to solublize the GLP-1 in the 1M NaCl samples and so gave lower ‘apparent’ loading. Precipitation was also observed in the 0M NaCl controls with 250 mM phosphate and iodide.

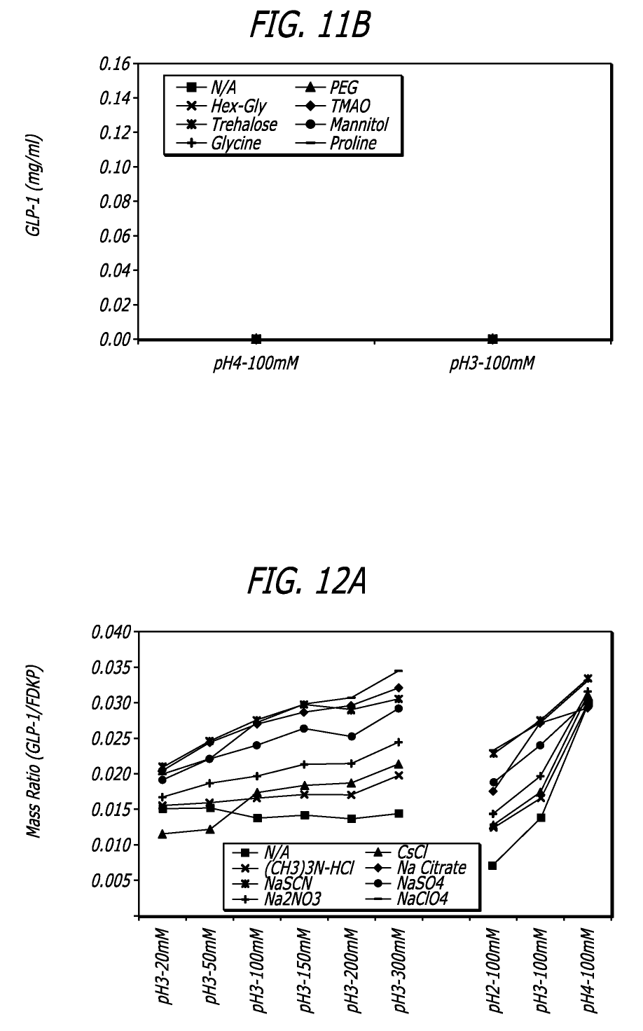

Osmolyte studies. The effect of osmolytes on the binding of GLP-1 to FDKP particles was also observed by HPLC analysis. FIG. 11A shows the loading curves for GLP-1/FDKP as a function of pH in the presence of common stabilizers (osmolytes). Loading of the GLP-1/FDKP particles was performed as described for the previous experiment. Similarly, the pH was controlled as described supra. The samples were prepared at pH 3.0 and in the presence of 20, 50, 100, 150, 200 or 300 mM of an osmolyte (stabilizer). The osmolytes were Hexylene-Glycol (Hex-Gly), trehalose, glycine, PEG, TMAO, mannitol or proline; N/A indicates no osmolyte. In a similar experiment, the concentration of the osmolyte (stabilizer) in the samples was held constant at 100 mM and the pH varied from 2.0 to 4.0.

None of the osmolytes (stabilizers) studied had a dramatic impact on GLP-1 adsorption to FDKP surfaces either when the pH was held at pH 3.0 and the concentrations of the osmolytes were varied (FIG. 11A; left hand curves) or when the osmolyte concentration was held constant at 100 mM and pH was varied (FIG. 11A; right hand curves). No precipitation of GLP-1 was detected in the reconstituted FDKP-free control samples (FIG. 11B). These osmolytes may be utilized to optimize stability and/or pharmacokinetics.

Chaotrope and Lyotrope studies. Ionic species that affect the structure of water and proteins (chaotropes and lyotropes) were studied to determine the role that these factors play in GLP-1 adsorption to FDKP. Loading of the GLP-1/FDKP particles was performed as described for the previous experiments. Similarly, the pH was controlled as described supra. The samples were prepared at pH 3.0 and in the presence of 0, 20, 50, 100, 150, 200 or 300 mM of the following chaotropes or lyotropes: NaSCN, CsCl, Na₂SO₄, (CH₃)₃N—HCl, Na₂NO₃, Na Citrate, and NaClO₄. In a similar experiment, the concentration of the chaotrope or lyotrope in the samples was held constant at 100 mM and the pH varied from 2.0 to 4.0.

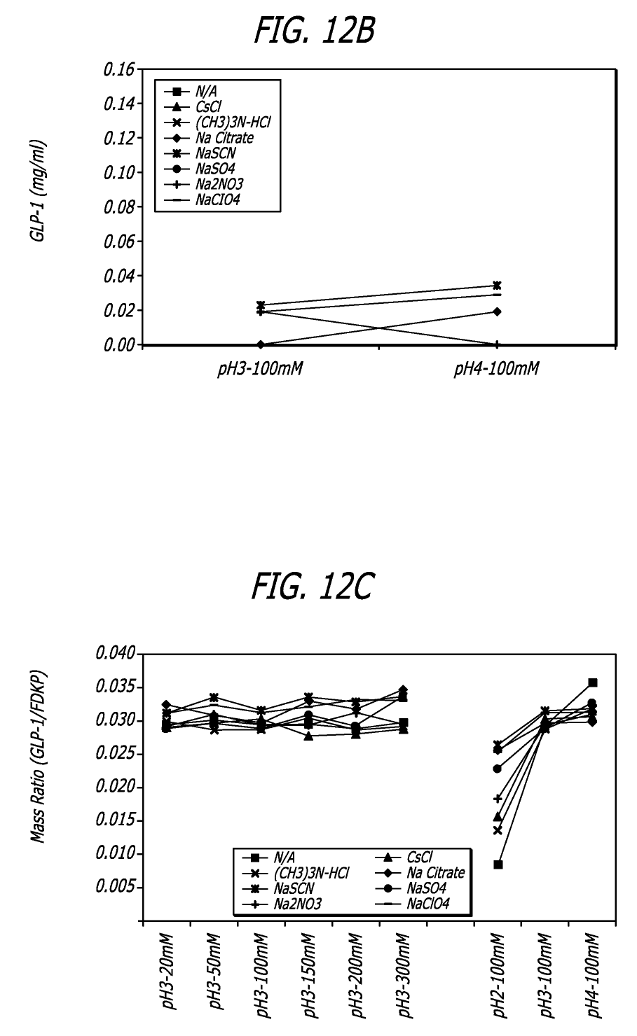

FIG. 12A shows the loading curves for GLP-1/FDKP as a function of pH and chaotrope and/or lyotrope. At low pH (≦3) significant variations in loading occurred for the different chaotropes analyzed, especially at higher chaotrope concentrations. However at pH 4, this variation was not observed (FIG. 12C). Thus, these agents appear to promote binding of GLP-1 to the FDKP particles at unfavorable lower pH, but have little impact at the higher pH conditions that are favorable to binding. The data from the reconstituted FDKP-free control samples suggests that the loading variations observed at pH 3.0 is due in part to specific chaotropes affecting the salting-out (precipitation) of GLP-1 peptide to various degrees (FIGS. 12B and 12D). This was noted for strong chaotropes such as NaSCN and NaClO₄.

Organic studies. Alcohols known to induce helical conformation in unstructured peptides by increasing hydrogen-bonding strength were evaluated to determine the role that helical confirmation plays in GLP-1 adsorption to FDKP. Loading of the GLP-1/FDKP particles was performed as described for the previous experiments. Similarly, the pH was controlled as described supra. The effects of each alcohol was observed at pH 2.0, 3.0, 4.0, and 5.0. The alcohols used were: methanol (MeOH), ethanol (EtOH), trifluoroethanol (TFE), or hexafluoroisopropanol (HFIP). Each alcohol was evaluated at a concentration of 5%, 10%, 15%, and 20% v/v.

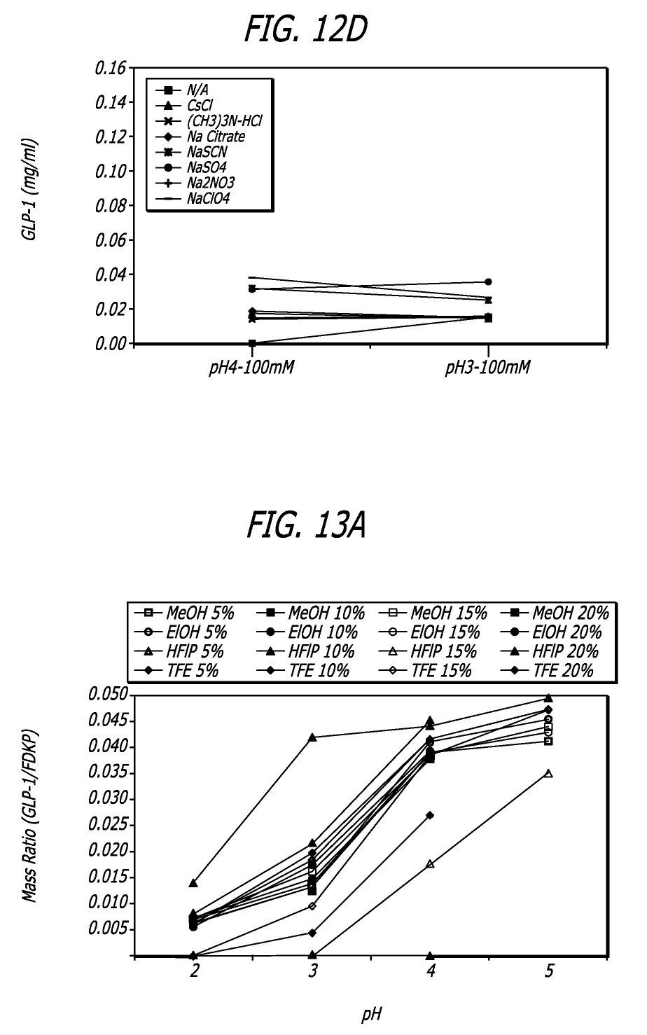

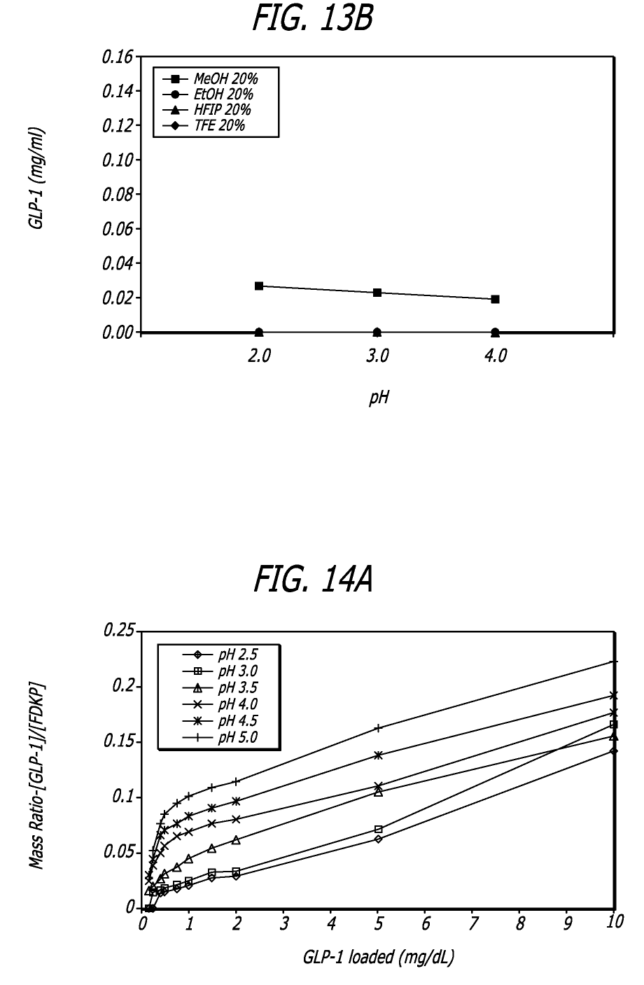

FIG. 13A shows the loading curves for GLP-1/FDKP as a function of pH for each alcohol at each concentration. At pH 3.0, low concentration of HFIP (5%) results in a high adsorption, as demonstrated by the mass ratio of GLP-1 to FDKP particles. Only the strongest H-bond strengthening (helix-forming) alcohol, HFIP, had an effect on adsorption in the buffered suspensions. At higher concentrations of HFIP (20%), GLP-1/FDKP adsorption was inhibited. FIG. 13B shows that at 20% alcohol concentration, no significant precipitation of GLP-1 was noted in the reconstituted FDKP-free control samples.

This suggests that conformational flexibility of a drug (i.e., entropy and the number of FDKP-contacts that can be formed) may play a role in adsorption. The data suggests that H-bonding may play a role in GLP-1 interaction with FDKP surfaces under the above conditions. Based on the data, it is further speculated that if H-bonding served as a dominant and a general force in FDKP-GLP-1 interactions, more and stronger effects would have been expected.

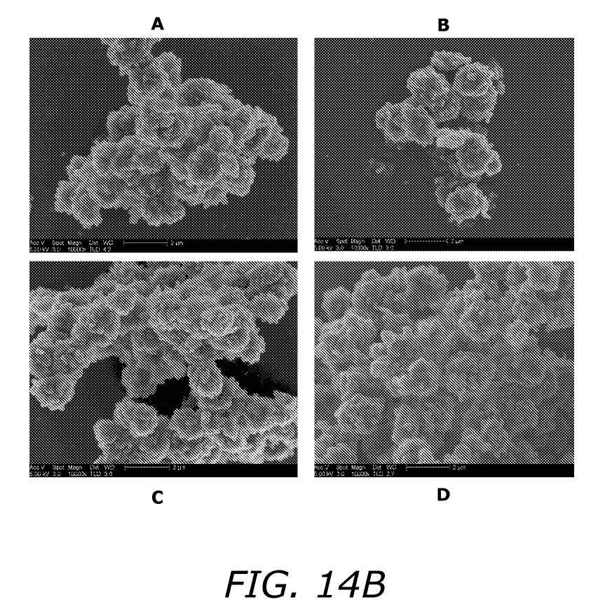

Concentration studies.—The adsorption of GLP-1 to FDKP particle surfaces at varying concentrations of GLP-1 was investigated. FIG. 14A shows loading curves from GLP-1/FDKP as a function of GLP-1 concentration at various pHs. GLP-1 concentrations were at 0.15, 0.25, 0.4, 0.5, 0.75, 1.0, 1.5, 2.0, 5.0, or 10 mg/mL. The pH of the samples was at 2.5, 3.0, 3.5, 4.0, 4.5 or 5.0.

An increase in GLP-1 loading on FDKP particles was observed when the FDKP concentration was held constant at 5 mg/mL and the GLP-1 concentration increased. Nearly 20% GLP-1 adsorption on FDKP particles was observed when the concentration of GLP-1 was 10 mg/mL at pH 4. Surprisingly, no saturation of adsorption of GLP-1 loading on FDKP particles was observed at high concentrations of GLP-1. This observation is probably attributable to the self association of GLP-1 into a multi-layer.

Analysis of the morphology of GLP-1/FDKP formulations by scanning electron microscopy (SEM) shows that GLP-1/FDKP particles are present as crystalline or plate like structures which can form aggregates comprising of more than one GLP-1/FDKP particles (FIG. 14B). These formulations were prepared by lyophilizing a solution containing: (Panel A) 0.5 mg/mL GLP-1 and 2.5 mg/mL FDKP; (Panel B) 0.5 mg/mL GLP-1 and 10 mg/mL FDKP; (Panel C) 0.5 mg/mL GLP-1 and 10 mg/mL FDKP in 20 mM sodium chloride, 20 mM potassium acetate and 20 mM potassium phosphate, pH 4.0; and (Panel D) 10 mg/mL GLP-1 and 50 mg/mL FDKP in 20 mM sodium chloride, 20 mM potassium acetate and 20 mM potassium phosphate at pH 4.0.

Summary of Results

Overall, the adsorption studies on the interaction of GLP-1 with FDKP particles showed that GLP-1 binds to the DKP particle surfaces in a pH-dependent manner, with high adsorption at pH 4 or above. The adsorption of GLP-1 to DKP particle surfaces was found to be most strongly affected by pH, with essentially no adsorption at pH 2.0 and substantial interaction at pH≧4.0. As observed, sodium and fluoride ions enhanced adsorption at low pH. Other additives such surfactants, and common stabilizers had only a slight effect on the adsorption of GLP-1 to FDKP particle surfaces.

In addition, the properties of GLP-1 itself influenced the results of these experiments. The behavior of GLP-1 was found to be atypical and surprising in that there was no saturation of adsorption observed, which was attributed to GLP-1 self-association at high concentrations. The self-association of GLP-1 at high concentration, allows for the possible coating of DKP particles with multiple layers of the GLP-1 peptide thereby promoting higher percent load of the GLP-1 peptide. This surprising self-association quality proves to be beneficial in preparing stable GLP-1 administration forms. Further, the self-associated conformation of GLP-1 may be able to lessen or delay its degradation in blood. However, it is noted that care must be taken when working with associated GLP-1 since it is sensitive to temperature and high pH.

### Example 3

### Integrity Analysis of GLP-1/FDKP Formulations

Based on the results from the experiments in Examples 1 and 2, a series of GLP-1 formulations having the characteristics described in Table 1 were selected for the cell viability assay as discussed herein. Most of the formulations contained GRAS (“generally recognized as safe”) excipients, but some were selected to allow the relationship between stability and adsorption to be studied.

TABLE 1
Selected GLP-1/FDKP Formulations for Integrity Phase Analysis.

| Modifier | Amount (mM) | No Buffer | pH 3.0 | pH 4.0 | pH 5.0 |
|---|---|---|---|---|---|
| None | | X | X | X | X |
| NaCl | 1000 | | X | X | |
| NaCl | 20 | | | X | |
| Tween 80 | 0.01% | | | X | |
| HepSulf | 0.90% | | | X | |
| Brij 78 | 0.09% | | | X | |
| F− | 250 | | | X | |
| F− | 20 | X | | X | |
| Li+ | 20 | X | X | X | |
| Phosphate | 250 | | X | X | |
| Phosphate | 20 | | X | X | X |
| Imidizole | 250 | | | X | |
| Mannitol | 20 | | | X | |
| Glycine | 20 | | | X | |
| Me₃N · HCl | 50 | | | X | |
| Citrate | 50 | | | X | |
| Am₂SO₄ | 50 | | | X | |
| ClO₄ | 50 | | | X | |
| EtOH | 20% | | | X | |
| TFE | 20% | | | X | |

Further, based on the results obtained in Examples 1 and 2, a series of formulations were also selected for phase II integrity studies of GLP-1/FDKP. Table 2 below shows the six formulations chosen for phase II integrity. After the powders were prepared, they were blended with blank FDKP to yield similar masses of both the GLP-1 peptide and FDKP in each formulation.

TABLE 2
GLP-1/FDKP formulations chosen for phase II integrity. The
formulation made from 10 mg/ml GLP-1 in 20 mM NaCl and pH 4.0
buffer is desribed as the salt-associated formulation.

| | | Mass ratio | Water | 20 mM NaCl + |
|---|---|---|---|---|
| GLP-1 Concentration | | (GLP/FDKP) | (no buffer) | pH 4.0 buffer |
| 0.5 | mg/mL | 0.05 | X | X |
| 3.0 | mg/mL | 0.10 | X | X |
| 10 | mg/mL | 0.20 | X | X |

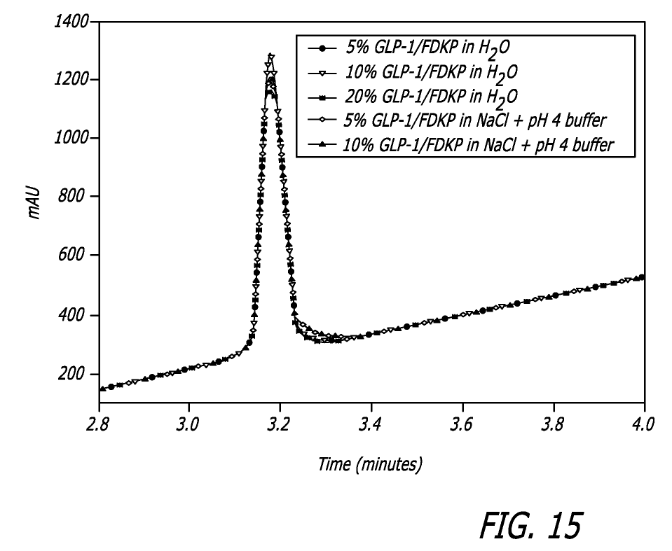

The effect of stress on the GLP-1/FDKP formulations in Table 2 was analyzed by HPLC (FIG. 15). The samples containing 5%, 10% or 20% GLP-1/FDKP loaded in H₂O; or 5%, or 10% GLP-1/FDKP loaded in NaCl+pH 4.0 buffer, were incubated for 10 days at 40° C. The HPLC chromatograms demonstrate that the GLP-1 peptide elutes at the same retention time and that no degradation peaks are present. Furthermore, MS analyses yielded a similar mass for all the samples, 3297 g/mol, indicating that the mass is uniform for all the samples analyzed. The data show the mass-to-mass ratio of GLP-1 to FDKP particles and the other components that were present in solution, prior to lyophilization. Overall, the GLP-1/FDKP formulations were shown to be stable to stress.

### Example 4

### Stability of GLP-1 Incubated in Lung Lavage Fluid

The stability of GLP-1 in biological fluids such as lung fluid and blood was analyzed given that dipeptidyl-peptidase IV (DPP-IV), found in biological fluids, cleaves and inactivates GLP-1.

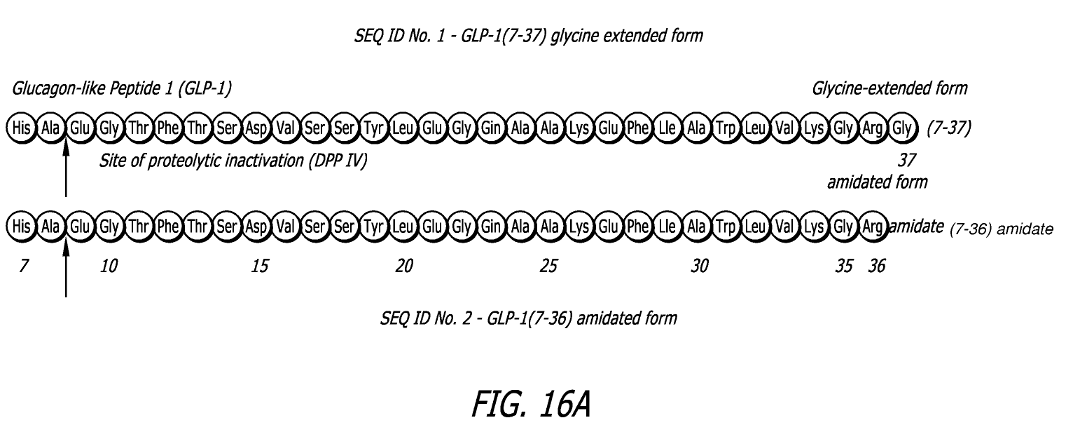

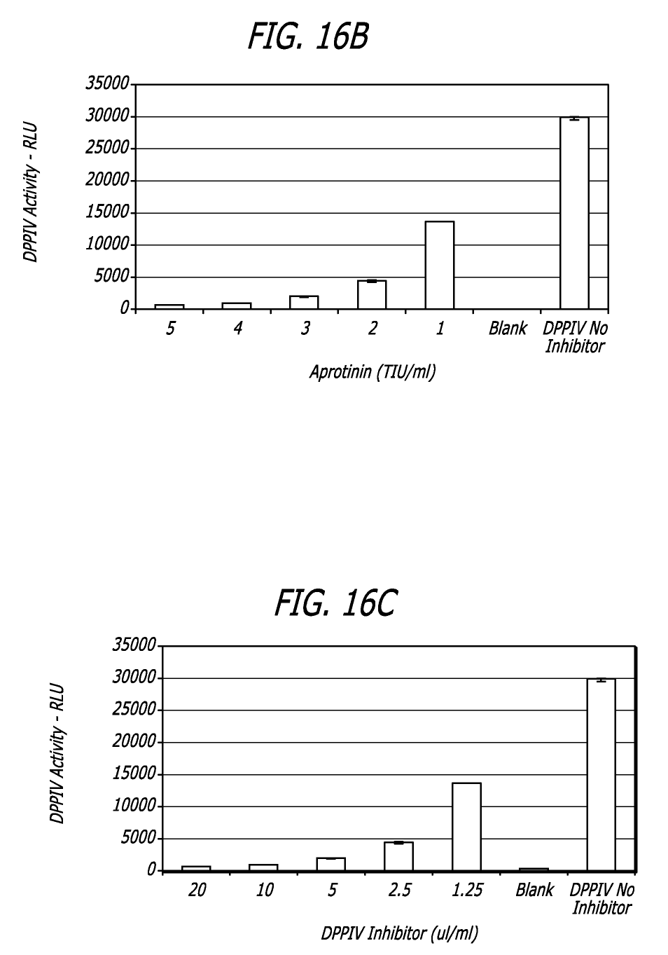

Dipeptidyl-peptidase IV (DPP-IV) is an extracellular membrane-bound serine protease, expressed on the surface of several cell types, in particular CD4⁺ T-cells. DPP-IV is also found is blood and lung fluids. DPP-IV has been implicated in the control of glucose metabolism because its substrates include the insulinotropic hormone GLP-1 which is inactivated by removal of its two N-terminal amino acids; see FIG. 16A. DPP-IV cleaves the Ala-Glu bond of the major circulating form of human GLP-1 (GLP-1 (7-36)) releasing the N-terminal two residues. DPP-IV exerts a negative regulation of glucose disposal by degrading GLP-1 thus lowering the incretin effect on β cells of the pancreas.

Studies were conducted to determine inhibition of GLP-1 degradation in rat blood and lung fluid in the presence of aprotinin or DPP-IV inhibitor. Aprotinin, a naturally occurring serine protease inhibitor, which is known in the art to inhibit protein degradation was added to the samples post collection at 1, 2, 3, 4 and 5 TIU/ml. DPP-IV activity was then measured by detecting the cleavage of a luminescent substrate containing the DPP-IV recognized Gly-Pro sequence. Bronchial lung lavage fluid was incubated with proluminescent substrate for 30 min and cleavage product was detected by luminescence.

The data showed an increase in inhibition of DPP-IV activity, as detected by the inhibition of peptide degradation in various biological fluids (as discussed herein) with increasing aprotinin concentration (FIG. 16B). Similar results were observed with DPP-IV inhibitor added to the samples post collection at 1.25, 2.5, 5, 10 and 20 μl/ml (FIG. 16C). Addition of inhibitors post-sample collection allowed for more accurate evaluation of the samples.

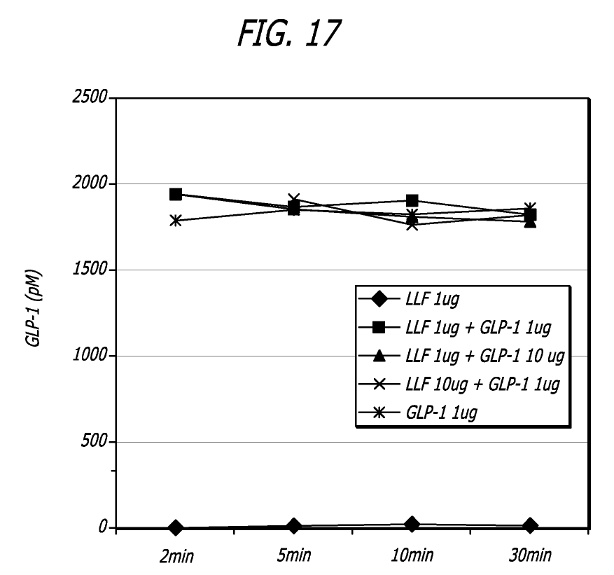

The stability of GLP-1 was also examined in lung lavage fluid using a capture ELISA mAb that recognizes GLP-1 amino acids 7-9. GLP-1 was incubated in lung lavage fluid (LLF) for 2, 5, 20 and 30 mins. The incubation conditions were: 1 or 10 μg (w/w) of LLF and 1 or 10 μg (w/w) GLP-1 as depicted in FIG. 17. No GLP-1 was detected in LLF alone. With the combination of LLF and GLP-1 at various concentrations there was a high detection of GLP-1 comparable to that of GLP-1 alone, indicating that GLP-1 is stable, over time, in lung lavage fluid (FIG. 17). Stability of GLP-1 in undiluted lung lavage fluid was confirmed in similar studies; at 20 minutes 70-72% GLP-1 integrity was noted (data not shown).

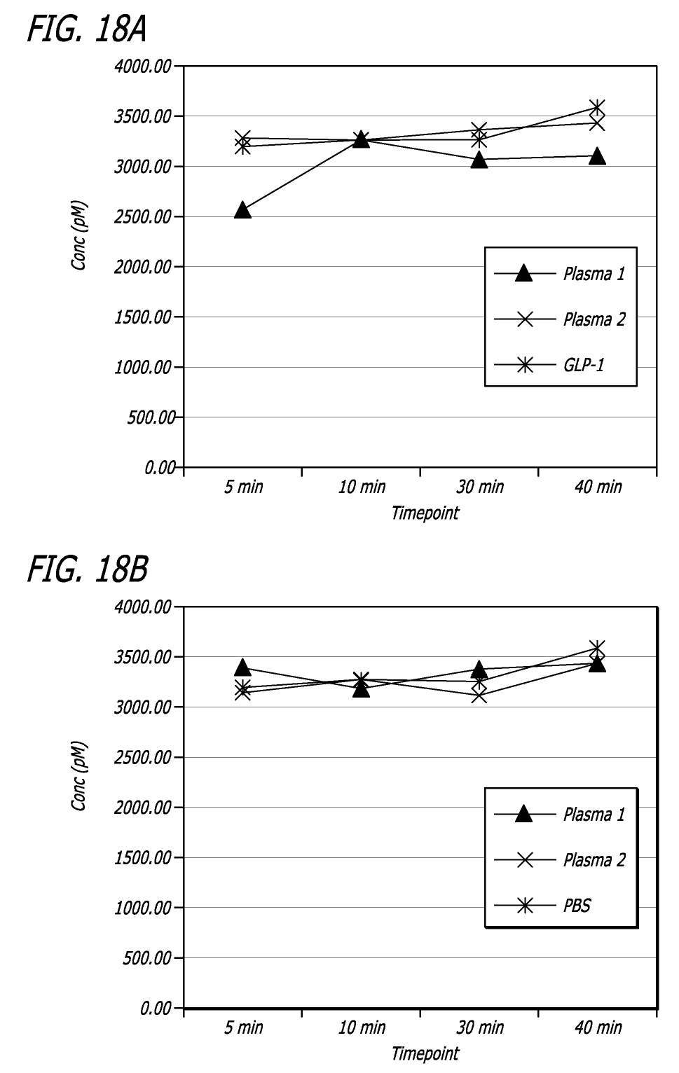

In addition, the stability of GLP-1 in rat plasma was examined. Plasma was obtained from various rats (as indicated by Plasma 1 and Plasma 2 in the figure legend) and diluted 1:2 or 1:10 (v/v). One microgram of GLP-1 was added to 10 μl plasma or PBS. Samples were incubated at 37° C. for 5, 10, 30 or 40 mins. The reaction was stopped on ice, and 0.1 U of aprotinin was added. The data shows a high concentration of GLP-1 in plasma dilutions 1:2 and 1:10 over all timepoints tested (FIGS. 18A and 18B). Overall, the data indicate that GLP-1 is surprisingly stable in both lung lavage fluid and plasma in which the serine protease DPP-IV is found.

### Example 5

### Effect of GLP-1 Molecules on Apoptosis and Cell Proliferation

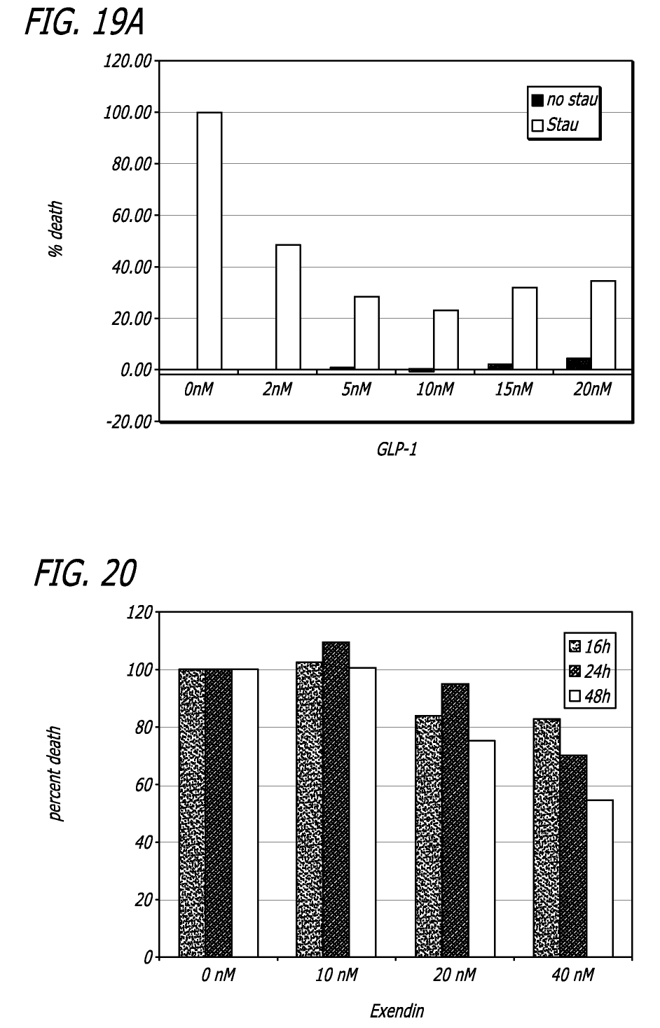

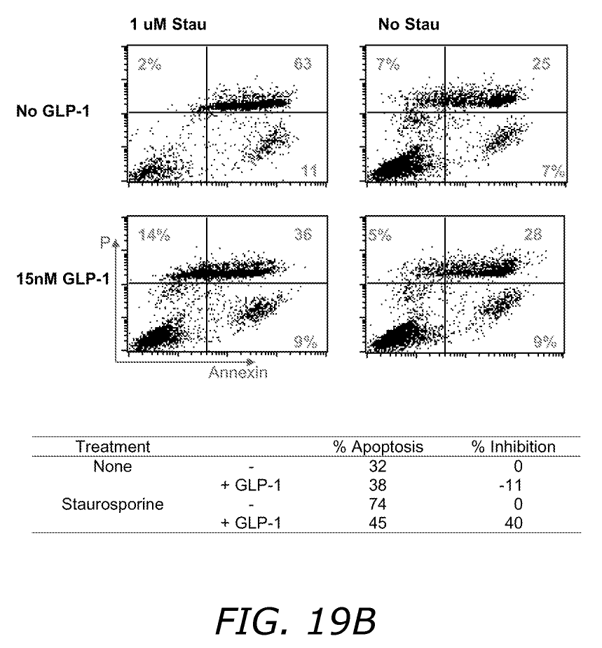

To examine whether GLP-1 inhibits apoptosis a screening assay was conducted to determine the effect of GLP-1 on inhibition of 1-cell death. Rat pancreatic epithelial (ARIP) cells (used as a pancreatic β-cell model; purchased from ATCC, Manassas, Va.) were pretreated with GLP-1 at 0, 2, 5, 10, 15 or 20 nM concentration for 10 minutes. The cells were then left untreated or were treated with 5 μM staurosporine (an apoptosis inducer) for 4.5 hours. Cell viability was evaluated using Cell Titer-Glo™ (Promega, Madison, Wis.). A decrease in the percent of cell death was noted with an increase in GLP-1 concentration of up to 10 nM in the staurosporine treated cells (FIG. 19A).

Further examination of the effect of GLP-1 on apoptosis was determined by FACS analysis using Annexin V staining. Annexin V staining is a useful tool in detecting apoptotic cells and is well known to those of skill in the art. Binding of Annexin V to the cell membrane, allows for the analysis of changes in phospholipids (PS) asymmetry before morphological changes associated with apoptosis occurred and before membrane integrity is lost. Thus, the effect of GLP-1 on apoptosis was determined in cells treated with 15 nm GLP-1, 1 μM staurosporine for 4 hrs, 1 μM staurosporine+15 nm GLP-1 or neither staurosporine nor GLP-1 (experimental control). The data shows that GLP-1 inhibited staurosporine induced apoptosis by about 40% (FIG. 19B).

Similar results of inhibition of apoptosis were observed using a GLP-1 analog, exendin-4, which binds to the GLP-1 receptor in a similar manner to GLP-1. ARIP cells were treated with 5 μM staurosporine in the presence of 0, 10, 20 or 40 nM exendin for 16, 24, or 48 hours respectively. The data (FIG. 20) shows that at 10 nM, exendin was completely ineffective at inhibiting apoptosis as there was 100% cell death. At 20 and 40 nM exendin inhibited apoptosis to some degree with about 50% inhibition at 48 hours in the presence of 40 nM of exendin-4.

### Example 6

### Effect of Candidate GLP-1/FDKP Formulations on Cell Death

Cell-based assays were conducted to assess the ability of GLP-1/FDKP formulations, (as disclosed in Example 3, Table 1 above), to inhibit cell death. These GLP-1/FDKP particle formulations were either in a suspension or lyophilized. The formulations were analyzed for their ability to inhibit staurosporine-induced cell death in ARIP cells. ARIP cells pre-treated with GLP-1 samples were exposed to 5 μM staurosporine for 4 hours and were analyzed with Cell Titer-Glo™ (Promega, Madison, Wis.) to determine cell viability.

Samples of the various GLP-1/FDKP formulations were either left unstressed or were stressed at 4° or 40° C. for 4 weeks. Each GLP-1/FDKP sample was used at 45 nM in a cell-based assay to determine their ability to inhibit stauorosporine induced cell death. Control samples, shown on the right, illustrate the viability of cells in media alone, with GLP-1 alone, with staurosporine alone, or in the presence of both GLP-1 and staurosporine (note: the graph legend does not apply to the control samples. Each bar represents a separate triplicate). All of the results shown are averages of triplicate runs.

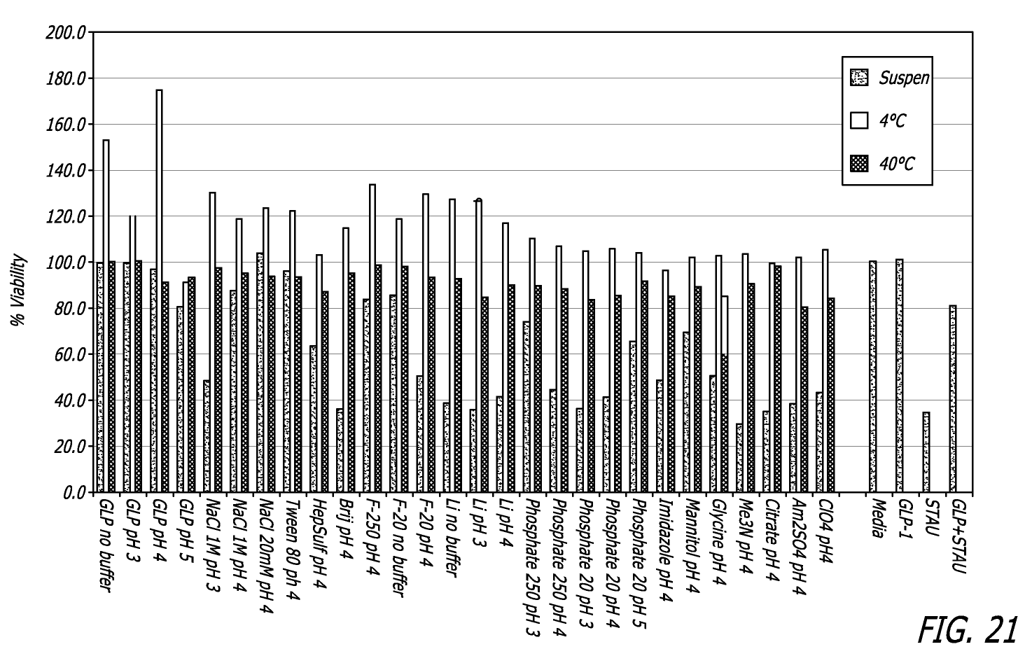

The data shows that all stressed GLP-1/FDKP lyophilized formulations inhibited staurosporine-induced cell death (FIG. 21). However, cell death was not inhibited by many of the GLP-1/FDKP suspension formulations.

### Example 7

### Pulmonary Insufflation of GLP-1/DKP Particles

To examine the pharmacokinetics of GLP-1/FDKP, plasma concentrations of GLP-1 were evaluated in female Sprague Dawley rats administered with various formulations of GLP-1/FDKP via intravenous injections or pulmonary insufflation. In the preliminary studies, GLP-1 at approximately 4% and 16% (w/w) of the GLP-1/FDKP particle formulations was used. Rats were randomized into 12 groups with groups 1, 4, 7 and 10 receiving GLP-1 solution administered via pulmonary liquid instillation or IV injection. Groups 2, 5, 8, and 11 received GLP-1/FDKP salt-associated formulation (as disclosed in Table 2), administered via pulmonary insufflation or IV injection. Groups 3, 6, 9, 12 received the GLP-1/FDKP salt-associated blended formulation administered via pulmonary insufflation or IV injection. The GLP-1/DKP formulation was a salt-associated formulation at approximately 16% load. To achieve an approximate 4% load, the 16% formulation was blended with DKP particles in a 3:1 mixture. Pulmonary insufflation or intravenous injection was at 0.5 or 2.0 mg of particles (16% or 4% GLP-1 load, respectively) for a total GLP-1 dose of 0.08 mg.

In a separate group of animals (Groups 7-12), administration was repeated on Day 2. Groups 1, 4, 7, and 10 were administered 80 μg of a GLP-1 solution. Groups 2, 5, 8, and 11 were administered a GLP-1/DKP salt-associated formulation (˜16% GLP-1 load). Groups 3, 6, 9, 12 received the GLP-1/DKP salt-associated blended formulation (˜4% GLP-1 load).

The experiment was performed twice using the same formulations, with dosing and blood collection on two consecutive days. Blood samples were taken on the day of dosing for each group at pre-dose (time 0), and at 2, 5, 10, 20, 30, 60 and 120 minutes post dose. At each time-point, approximately 150 μL whole blood was collected from the lateral tail vein into a cyro-vial tube containing approximately 3 U/mL aprotinin and 0.3% EDTA, inverted and stored on ice. Blood samples were centrifuged at 4000 rpm and 40 μl of plasma was pipetted into 96-well plates which were stored at −80° C. until analyzed for GLP-1 levels by ELISA following manufactures' recommendations (Linco Research, St Charles, Mo.). It was determined that the optimal conditions were when the assay buffer was GLP-1 in the presence of serum (5% FBS) alone and no matrix.

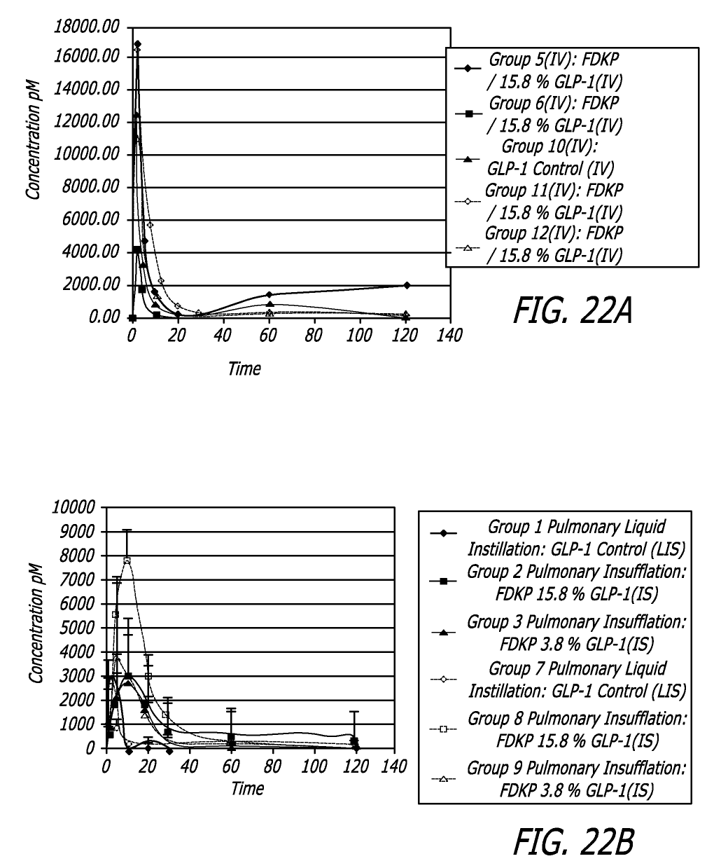

Intravenous Administration: Groups 5, 6, 10, 11 and 12 received various GLP-1/FDKP formulations and GLP-1 solution intravenously (IV); (FIG. 22A). Groups 5 and 6 were administered 15.8% GLP-1/FDKP and groups 11 and 12 were administered another dose of 15.8% GLP-1/FDKP on a consecutive day; group 10 was administered GLP-1 solution as a control. The concentration of GLP-1/FDKP was detected at time points of 0, 2, 5, 10, 20, 40, 60, 80, 100, and 120 mins. All groups showed a detectable increase in GLP-1 plasma levels after intravenous administration, with maximal concentrations observed at 2 minutes post treatment. Plasma levels of active GLP-1 returned to background levels by 20 minutes post treatment for all groups. No significant difference was observed in the kinetics of these various formulations of GLP-1/FDKP and GLP-1 solution when administered by intravenous injection. It was noted that plasma levels of GLP-1 returned to baseline levels at 10-20 minutes post dose in rats treated via intravenous injections suggesting physiological kinetics (i.e., about 95% of GLP-1 was eliminated within 10 mins).

Single Insufflation Administration: Groups 1, 2, 3, 7, 8 and 9 12 received various GLP-1/FDKP formulations or GLP-1 solution by pulmonary insufflation (FIG. 22B). Group 1 was administered 80 μg of a GLP-1 control by pulmonary liquid instillation (LIS); group 2 was administered 15.8% GLP-1/FDKP by pulmonary insufflation (IS); group 3 was administered 3.8% GLP-1/FDKP by pulmonary insufflation (IS); group 7 was administered 80 μg of a GLP-1 control by pulmonary liquid instillation (LIS); group 8 was administered 15.8% GLP-1/FDKP by pulmonary insufflation (IS); and group 9 was administered 3.8% GLP-1/FDKP by pulmonary insufflation (IS). The concentration of GLP-1/FDKP was measured at time points of 0, 2, 5, 10, 20, 40, 60, 80, 100, and 120 mins.

All groups showed a detectable increase in plasma GLP-1 concentration following pulmonary administration. Maximum plasma concentration of GLP-1 varied with the formulation/composition used. Groups 2 and 8 showed maximal plasma levels of GLP-1 at 10-20 minutes post treatment as indicated by the AUC, while groups 3 and 9 showed significant levels of active GLP-1 at 5-10 minutes, and groups 1 and 7 showed a more rapid and transient increase in plasma levels of active GLP-1. Plasma levels of active GLP-1 returned to background levels by 60 minutes post treatment in groups 2, 3, 7 and 8, while groups 1 and 7 reached background levels by 20 minutes post treatment.

Eight nanomolar GLP-1 appears to be efficacious in a diabetic rat model; the GLP-1 dose was 80 μg (3000-fold greater than the reported efficacious dose); plasma GLP-1 levels were 10-fold greater with pulmonary delivery versus a 3 hr infusion (Chelikani et al., 2005) at 30 minutes post dose; and the bioavailability of GLP-1/FDKP delivered via pulmonary insufflation was 71%. These results are further reported in Table 4 below. Plasma levels of GLP-1 returned to baseline levels at 30-60 minutes post dose in most rats treated via pulmonary delivery. All rats showed an increase in plasma concentrations of GLP-1 after intravenous administration or pulmonary insufflation of various GLP-1/FDKP formulations, except for 1 rat in group 2.

Conclusion: A difference was observed in the pharmacokinetics profiles of GLP-1/FDKP formulations compared to GLP-1 solution. Plasma concentrations of GLP-1 were more sustained in rats treated by pulmonary insufflation with GLP-1/FDKP formulations relative to those treated with GLP-1 solution. All animals showed a progressive decrease in plasma concentrations of GLP-1 between 20 and 60 minutes post dose. These results showed relative consistency in 2 experiments performed on 2 consecutive days.

TABLE 4
Bioavailability of GLP-1/FDKP Formulations

| | | GLP-1 | | | | | 30 min | |
|---|---|---|---|---|---|---|---|---|
| | | Dose | | T_{1/2} | Tₘₐₓ | Cₘₐₓ | post dose | AUC |
| Group | Formulations | (μg)/μM | Route | (min) | (min) | (pM) | (~pM) | (pM*min/mL) |
| 1 | GLP-1 | 80/24 | LIS | 1.0 | 5 | 1933 | 0 | 29350 |
| 2 | FDKP-GLP-1 | 80/24 | IS | 9.9 | 10 | 3154 | 1000 | 145082 |
| 3 | FDKP-GLP-1* | 80/24 | IS | 7.7 | 10 | 2776 | 400 | 60171 |

*Blended 3:1 with FDKP particles

### Example 8

### GLP-1/FDKP Reduces Food Intake in Rats

GLP-1 is also known in the art to work in the brain to trigger a feeling of satiety and reduce food intake. Based on this role of GLP-1 in satiety and reduction of food intake, experiments were conducted to determine whether GLP-1/FDKP formulations of the present invention were effective as agents to reduce feeding and thereby have potential for controlling obesity.

Two groups of female Sprague Dawley rats were dosed with either a control (air) or 15.8% GLP-1/FDKP formulation at a dosage of 2 mg/day (0.32 mg GLP-1/dose) by pulmonary insufflation. The control group consisted of five rats and the test group consisted of ten rats. Each rat was provided with a single dose for 5 consecutive days and the food intake measured 2 and 6 hours following each dose. The body weight of each rat was collected daily.

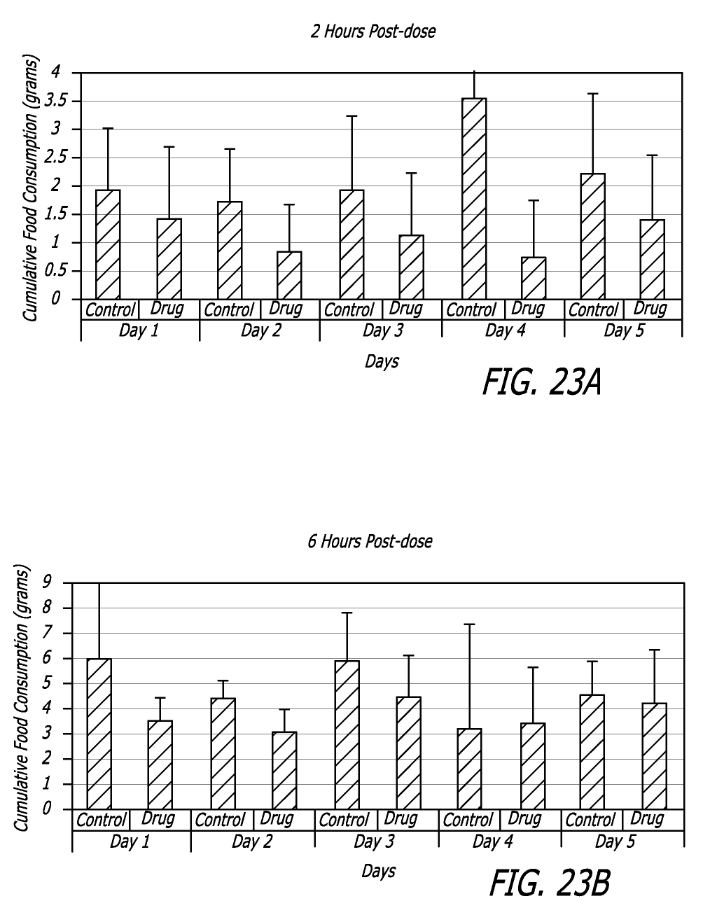

The preliminary data shows that at 2 and 6 hours post dose, there was an overall decrease in the cumulative food consumption in the rats dosed with GLP-1/FDKP formulations (FIGS. 23A and 23B). The decrease was more pronounced at day 4 at 2 hours post dosing (p=0.01). At 6 hours the decrease was more pronounced at days 1 and 2 (p<0.02). There was no effect on food consumption at 24 hours post dose.

### Example 9

### Toxicity Studies

Repeat dose toxicity studies to evaluate the potential toxic effects and toxicokinetic profile of GLP-1/DKP after multiple administrations were conducted. Fourteen day study in rats and a twenty-eight day study in monkeys was performed. GLP-1/DKP will be dosed daily, via the inhalation route. In studies where animals were dosed for 28 days, a proportion of the animals will be sacrificed immediately after the dosing regimen while other animals will be allowed up to a one month recovery period prior to sacrifice. All animals will be evaluated for clinical signs, various physiological parameters including GLP-1, glucose, insulin, organ weights, and clinical pathology and histopathology of various organs.

A series of GLP mutagenicity studies were performed to evaluate the mutagenic potential of diketopiperazine particles. These studies included the in vitro Ames and Chromosomal aberration assays, both which are well known to those of skill in the art. In addition, an in vivo mouse micronucleus assay, as is known to the skilled artisan, was also conducted. The genotoxicity data shows that there was no evidence of potential for mutagenicity or genetic toxicity with diketopiperazine particles.

Studies were also conducted to assess the effect of diketopiperazine particles on reproductive toxicity. These studies included fertility, embryo-fetal development and postnatal development studies in rats and rabbits. Diketopiperazine particles administered via subcuteanous injection does not impair fertility or implantation in rats and there is no evidence of teratogenicity in rats or rabbits. Diketopiperazine particles did not adversely affect fertility and early embryonic development, embryo fetal development, or prenatal or postnatal development.

Given that a number of pharmaceuticals have been removed from the clinical market due to their propensity to cause LQT syndrome (acquired LQTS or Long Q-T syndrome is an infrequent, hereditary disorder of the heart's electrical rhythm that can occur in otherwise-healthy people) an hERG assay was employed to examine the pharmacology of diketopiperazine particles. The hERG assay was utilized given that the vast majority of pharmaceuticals that cause acquired LQTS do so by blocking the human ether-à-go-go related gene (hERG) potassium channel that is responsible for the repolarization of the ventricular cardiac action potential. Results from the hERG assay indicated an IC₅₀>100 μM for diketopiperazine particles. In addition, results from nonclinical studies with diketopiperazine particles showed no effect on the QTc interval (the heart rate-corrected QT interval) as prolongation was not observed in the dog (9-month or safety pharmacology cardiovascular studies). There were no effects of diketopiperazine particles, when administered intravenously, on CNS or cardiovascular systems evaluated in the safety pharmacology core battery.

### Example 10

### Effect of GLP-1 on β-Cell Mass

GLP-1 is known to promote all steps in insulin biosynthesis and directly stimulate β-cell growth and survival as well as β-cell differentiation. The combination of these effects results in increased β-cell mass. Furthermore, GLP-1 receptor signaling results in a reduction of β-cell apoptosis, which further contributes to increased β-cell mass. GLP-1 is known to modulate β-cell mass by three potential pathways: enhancement of β-cell proliferation; inhibition of apoptosis of β-cells; and differentiation of putative stem cells in the ductal epithelium.

To demonstrate the effect of GLP-1 on β-cell mass, cells were treated at day 1, 3 and 5 with GLP-1/FDKP and compared to untreated cells. Administration of active GLP-1 increased β-cell mass by up to 2-fold as suggested in the literature (Sturis et al., 2003). In addition, examination of the effect of various GLP-1 receptor (GLP-1R) agonists on diabetes demonstrated that GLP-1R agonists prevent or delay occurrence or progression of diabetes.

The effects of GLP-1/FDKP on β cell proliferation, insulin and glucose were assessed in male Zucker Diabetic Fatty/Obese (ZDF) rats (n=8/group). Animals received either control (air) or 2 mg GLP-1/FDKP containing 15% (0.3 mg) GLP-1 daily for 3 consecutive days. An intraperitoneal (IP) glucose tolerance test was conducted and blood samples were collected for plasma GLP-1 and glucose analysis pre-dose, and at 15, 30, 45, 60 and 90 minutes post-dose. Pancreatic tissues were collected for insulin secretion, β cell mass, and apoptosis analysis via immunohistochemistry.

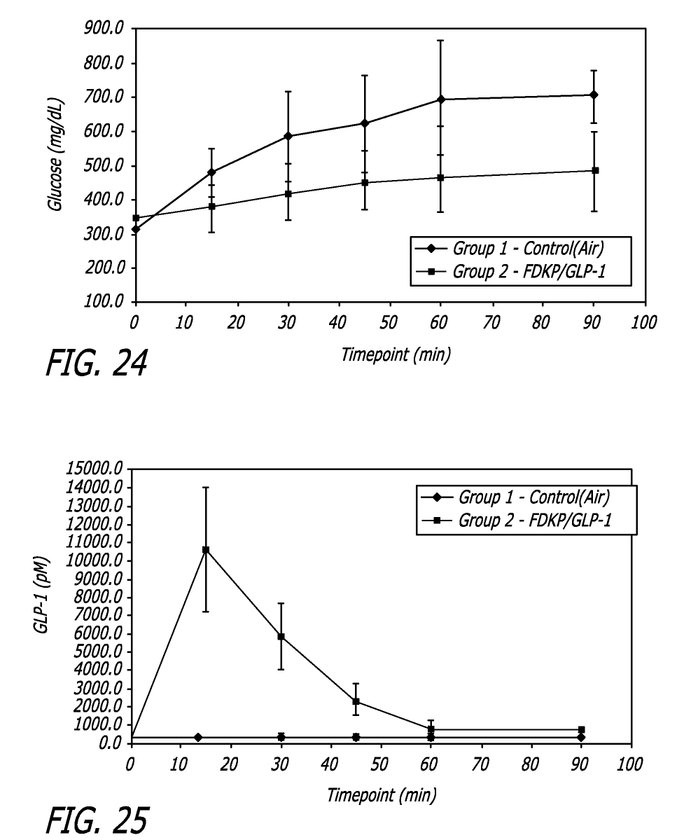

An IP glucose tolerance test (IPGTT, FIG. 24) was conducted on day 4 of dosing. After an overnight fast, on day 3, animals received a glucose bolus via intraperitoneal injection followed immediately by control (air) or GLP-1/FDKP administration via pulmonary insufflation. Blood was collected prior to the glucose challenge and at various timepoints out to 90 minutes post dose. At 30 minutes post-dose, Group 1 showed a 47% increase in glucose levels compared to predose whereas Group 2 (GLP-1/FDKP) showed a 17% increase in glucose levels compared to predose values. Glucose levels were significantly lower across all timepoints following the glucose tolerance test in the treated versus control animals (p<0.05).

GLP-1 levels were also measured on day 3 of dosing (FIG. 25). The maximum concentration of plasma GLP-1 levels in Group 2 was 10,643 μM at 15 minutes post-dose.

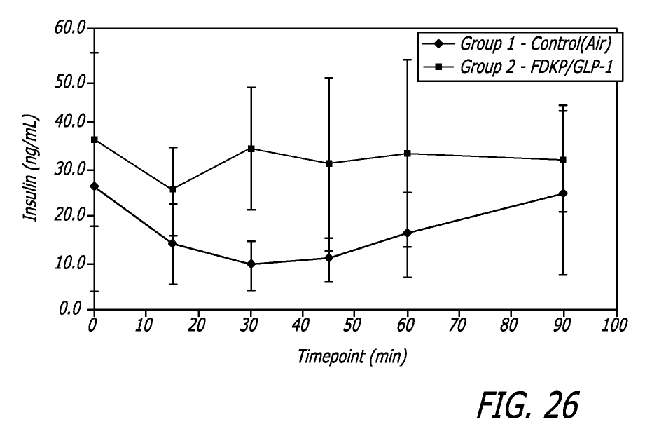

In addition, insulin levels were measured at various timepoints on day 3 along with glucose measurements following the IP glucose tolerance test. Both control (air) Group 1 and Group 2 (GLP-/DKP) demonstrated an initial decrease in insulin concentration from pre-dose levels, 46% and 30%, respectively, by 15 minutes post-dose (FIG. 26). However, at 30 minutes post-dose, insulin levels in Group 2 returned to baseline whereas insulin levels in Group 1 continued to decrease to 64% of pre-dose values. In treated animals, insulin levels at 45 minutes, 60 minutes, and 90 minutes were near pre-dose values with deviations of less than 1.5%.

Slides were prepared for insulin immunostaining and microscopic evaluation of insulin expression. Based on quantitative assessment of insulin expression by light microscopy, there was a treatment-related increase in insulin expression within the pancreas of male ZDF rats that was dose-related, although statistical significance was not attained (p=0.067); as determined by the percentage of β islet cells expressing insulin.

Apoptosis analysis was also conducted on the pancreatic tissue of ZDF rats. Exocrine and endocrine pancreas cells were evaluated by the TUNEL assay (Tornusciolo D. R. et al., 1995). Approximately 10,000 cells in the pancreas (exocrine and endocrine) were scored. Most TUNEL-positive cells were exocrine. There were no differences in apoptosis labeling index in treated versus control groups.

In addition, β cell proliferation was evaluated in the pancreas of Zucker Diabetic obese rats dosed once daily for 3 days with control (air) or GLP-1/FDKP via pulmonary insufflation. Slides were prepared for co-localization of insulin and Ki67 (a proliferation marker) using immunohistochemistry. Microscopic evaluation of cell proliferation was conducted within insulin-positive islets and in the exocrine pancreas in a total of 17 ZDF rats. Based on quantitative assessment of cell proliferation, there were no treatment-related effects on cell proliferation within the islet beta cells or exocrine cells of the pancreas in male ZDF rats.

Overall, this study shows that GLP-1/FDKP administered at 2 mg or 0.3 mg GLP-1 via pulmonary insufflation lowered blood glucose levels in diabetic fatty rats (model for Type 2 diabetes) following a glucose tolerance test and increased the number of insulin secreting cells per islet.

### Example 11

### Preparation of GLP-1/FDKP Particle Formulations

An alternative methodology for preparing GLP-1/FDKP particle formulations was also employed. The formulations were prepared as follows: A 10 wt % GLP-1 stock solution was prepared by adding 1 part GLP-1 (by weight) to 9 parts deionized water and adding a small amount of glacial acetic acid to obtain a clear solution. A stock suspension of FDKP particles (approximately 10 wt % particles) was divided into three portions. An appropriate amount of GLP-1 stock solution was added to each suspension to provide target compositions of 5 and 15 wt % GLP-1 in the dried powder. After addition of the protein solution, the pH of the suspensions was approximately 3.5. The suspensions were then adjusted to approximately pH 4.4-4.5, after which the suspensions were pelletized in liquid nitrogen and lyophilized to remove the ice.

The aerodynamics of the powders is characterized in terms of respirable fraction on fill (RF Based on Fill), i.e., the percentage (%) of powder in the respirable range normalized by the quantity of powder in the cartridge, which was determined as follows: five cartridges were manually filled with 5 mg of powder and discharged through MannKind's MedTone® inhaler (described in U.S. patent application Ser. No. 10/655,153).

This methodology produced a formulation with a good RF on fill. The powder with 5 wt % GLP-1 was measured at 48.8% RF/fill while the powder containing approximately 15 wt % GLP-1 was 32.2% RF/fill.

### Example 12

### Pharmacokinetics of GLP-1/FDKP with Various GLP-1 Concentrations

To assess the pharmacokinetic properties of GLP-1/FDKP with various concentrations of GLP-1, eighteen female Sprague Dawley rats weighing between 192.3 grams to 211.5 grams were divided into four treatment groups: Control GLP-1 (Group 1, n=3); GLP-1/FDKP formulations (Groups 2-4, n=5/group). Animals received one of the following test articles: control (air) via pulmonary instillation; 2.42 mg GLP-1/FDKP containing 5% GLP-1 (0.12 mg GLP-1); 1.85 mg GLP-1/FDKP containing 10% GLP-1 (0.19 mg GLP-1), or 2.46 mg GLP-1/FDKP containing 15% GLP-1 (0.37 mg GLP-1) via pulmonary insufflation. Blood samples were collected and assayed for serum FDKP and plasma GLP-1 levels predose and at various timepoints (2, 5, 10, 20, 30, 40 and 60 minutes) post dose.

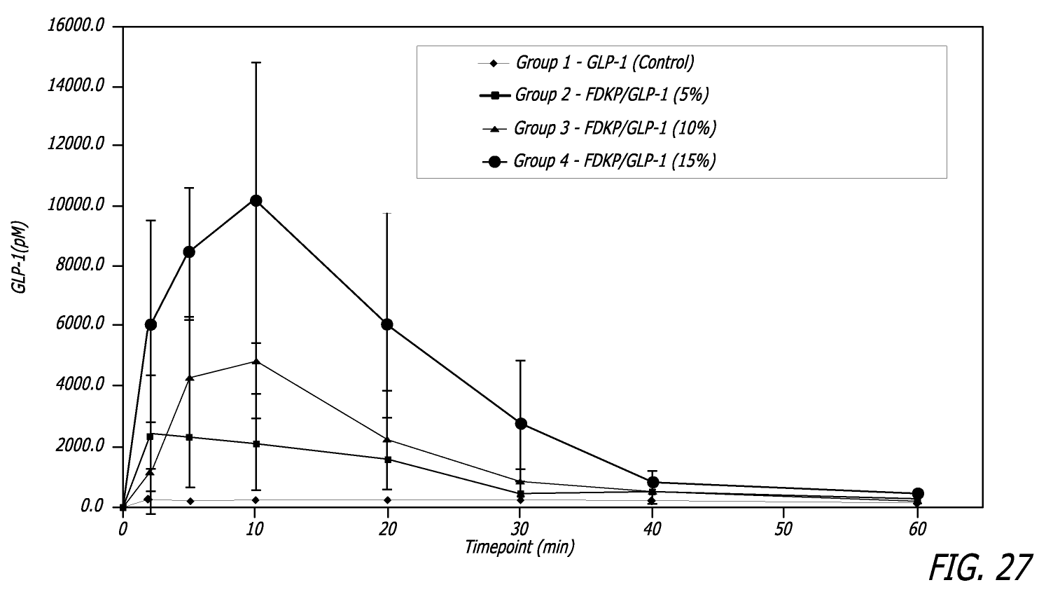

The maximum plasma GLP-1 concentrations (Cₘₐₓ) following the administration of GLP-1/FDKP (5% formulation) were 2321 pM at a Tₘₐₓ of 5 minutes post dose; 4,887 pM at a Tₘₐₓ of 10 minutes post dose (10% formulation); and 10,207 pM at a Tₘₐₓ of 10 minutes post dose (15% formulation). As depicted in FIG. 27 significant GLP-1 levels out to 30 minutes post dose was observed. The area under the curve (AUC) levels for GLP-1 were 10622, 57101, 92606, 227873 pM*min for Groups 1-4, respectively. Estimated half-life of GLP-1 was 10 min for GLP-1/FDKP at 10% or 15% GLP-1 load.

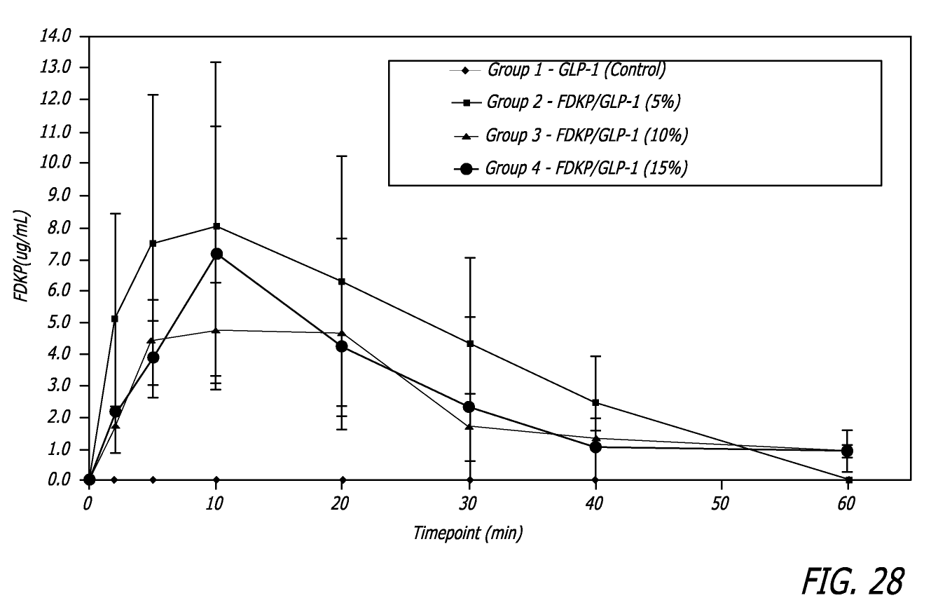

As depicted in FIG. 28, maximum FDKP concentrations were determined to be 8.5 μg/mL (Group 2), 4.8 μg/mL (Group 3) and 7.1 μg/mL (Group 4) for the GLP-1/FDKP formulations at 5%, 10% and 15% GLP-1, respectively. The time to maximum concentrations (Tₘₐₓ) was 10 minutes. This data shows that, FDKP and GLP-1 exhibited similar absorption kinetics and similar amounts of FDKP were absorbed independent of the GLP-1 load on the particles.

Overall, the study showed that plasma GLP-1 levels were detected at significant levels after single dose administration of GLP-1/FDKP via pulmonary insufflation in Sprague Dawley rats. Dose related increases in plasma GLP-1 levels were observed with maximum concentrations achieved at approximately 10 min post dose and with observable GLP-1 levels at 40 minutes post dose. All animals survived until the completion of the study.

### Example 13

### Pharmacodynamic Properties of GLP-1/FDKP Administered Via Pulmonary Insufflation

To assess the pharmacodynamic properties of GLP-1/FDKP, female Sprague Dawley rats were divided into 2 treatment groups. Animals received either control (air; n=5) or 2 mg GLP-1/FDKP containing 15% GLP-1 (0.3 mg GLP-1) via a single daily pulmonary insufflation (n=10) for 4 consecutive days.

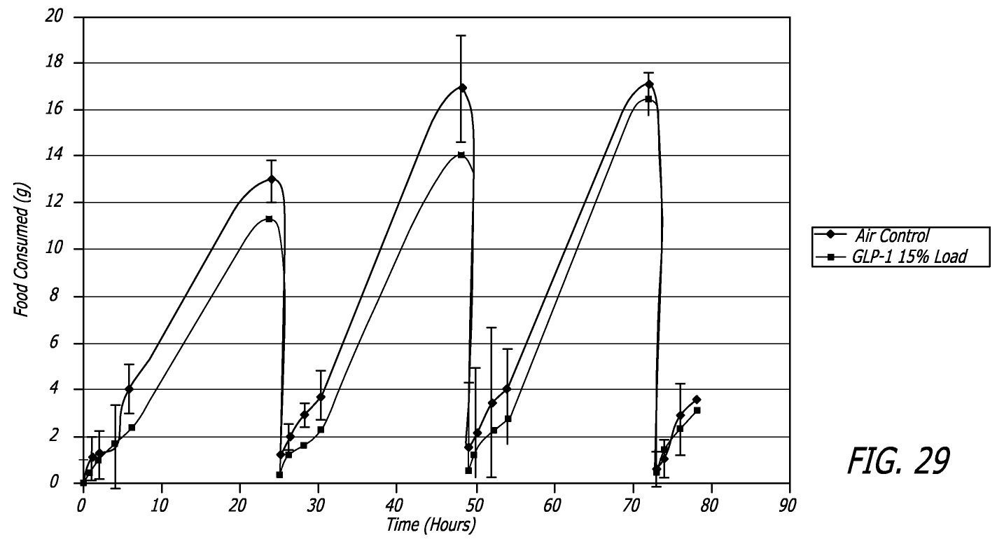

Food consumption was measured during the dark cycle at predose, 1, 2, 4 and 6 hours post dose for 4 consecutive days (FIG. 29). Food consumption was decreased on Days 1, 2 and 3 after daily single dose administration of GLP-1/FDKP via pulmonary insufflation in the treated animals compared to the control (air) group (p<0.05). There were statistically significant decreases in food consumption for animals in the treated group versus control (air) on Day 1 at the 1 hour and 6 hour timepoint and on Day 2 at the 4 hour, 6 hour and at predose on Day 3.

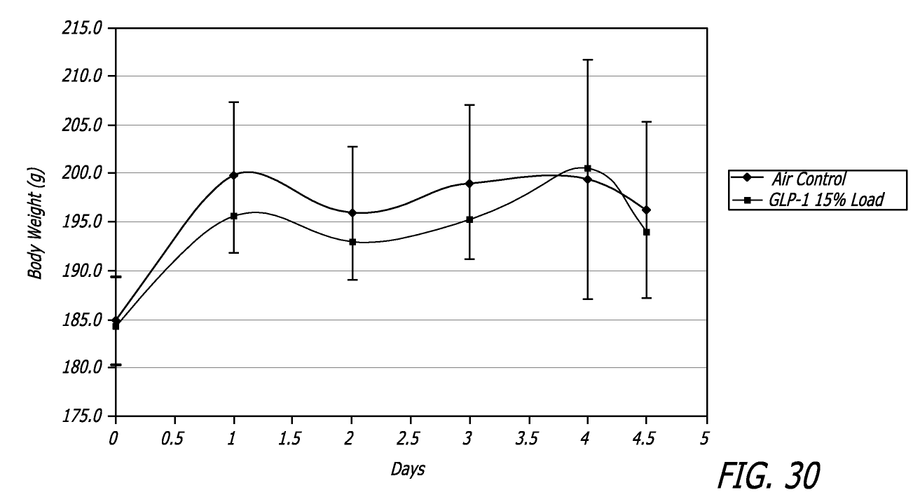

Body weights (FIG. 30) were measured daily at predose for 4 consecutive days. Body weights at the initiation of dosing ranged from about 180 to 209 grams. Although statistical significance between treated and control (air) animals was not reached, body weight were lower in treated animals. All animals survived until scheduled sacrifice.

### Examples 14-16

### Toxicokinetics (TK) Studies

### Examples 14 to 16 below disclose repeat-dose toxicity studies performed in rats and monkeys to evaluate the potential toxic effects and toxicokinetic profile of GLP-1/FDKP inhalation powder. The data indicates no apparent toxicity with GLP-1/FDKP inhalation powder at doses several fold higher than those proposed for clinical use. Additionally, there appeared to be no differences between the male and female animals within each species.

### Example 14

### Toxicokinetics of GLP-1/FDKP Administered for 5 Days Via Pulmonary Insufflation in Monkeys

Studies were conducted to determine the toxicity and toxicokinetic profile of GLP-1/FDKP via oronasal administration (the intended human therapeutic route of administration) to the cynomolgus monkey (Macaca fascicularis), once daily (for 30 minutes a day) for 5 consecutive days. Oronasal administration involved the monkeys wearing a mask over their mouth and nose and breathing the test formulation for 30 min.

Fourteen days prior to the start of treatment, the animals were acclimated to the restraint and dosing procedures. At the start of treatment (Day 1), male animals were between 30 months and 56 months old and ranged in weight from 2.3 to 4.0 kg; females were between 31 months and 64 months and ranged in weight from 1.6 to 3.4 kg. Ten (5 male and 5 female) non-naive cynomolgus monkeys were assigned to 5 groups (2 animals per group) as depicted in tables 5 and 6 below. The non-naive monkeys are colony animals who have previously received the formulations to be tested. However, these formulations have short half lives and are not expected to be present or have any effect on the monkeys during the dosing experiments disclosed herein. Animals received control (air), 2 mg/kg FDKP or 0.3 (0.04 mg GLP-1), 1.0 (0.13 mg GLP-1), or 2.0 (0.26 mg GLP-1) mg/kg GLP-1/FDKP.

TABLE 5
Targeted and estimated achieved mean dose levels
(determined by gravimetric analysis*):

| | | Estimated Dose Level (mg/kg/day) | | | | | |
|---|---|---|---|---|---|---|---|
| Group | Group | FDKP ³ | | GLP-1 ³ | | GLP-1/FDKP | |
| Number | Designation | Target ¹ | Achieved ² | Target ¹ | Achieved ² | Target ¹ | Achieved ² |
| 1 | Air Control | 0 | 0 | 0 | 0 | 0 | 0 |
| 2 | Vehicle | 2.00 | 2.10 | 0 | 0 | 2.0 | 2.10 |
| | Control | | | | | | |
| 3 | Low Dose | 0.26 | 0.31 | 0.04 | 0.05 | 0.3 | 0.35 |
| 4 | Mid Dose | 0.87 | 0.81 | 0.13 | 0.14 | 1.0 | 0.93 |
| 5 | High Dose | 1.74 | 1.85 | 0.26 | 0.28 | 2.0 | 2.13 |

*Gravimetric analysis is performed by weighing the filter papers in the inhalation chamber both before, during and after dosing to calculate the aerosol concentration in the chamber and to determine the duration of dosing.
¹ Based on an assumed body weight of 2.5 kg.
² Based on the measured body weights (average for male and female).
³ The targeted and achieved dose levels quoted assume that the proportion of GLP-1 in the generated atmosphere is 13%. The estimation of total inhaled dose assumed 100% deposition within the respiratory tract.

TABLE 6
Targeted and achieved mean aerosol concentrations
(determined by gravimetric analysis*):

| | | Aerosol Concentration (mg/L) | | | | | |
|---|---|---|---|---|---|---|---|
| Group | Group | FDKP ¹ | | GLP-1 ¹ | | GLP-1/FDKP | |
| Number | Designation | Target | Achieved | Target | Achieved | Target | Achieved |
| 1 | Air Control | 0 | 0 | 0 | 0 | 0 | 0 |
| 2 | Vehicle | 0.160 | 0.189 | 0 | 0 | 0.160 | 0.189 |
| | Control | | | | | | |
| 3 | Low Dose | 0.021 | 0.027 | 0.003 | 0.004 | 0.024 | 0.031 |
| 4 | Mid Dose | 0.070 | 0.073 | 0.010 | 0.011 | 0.080 | 0.084 |
| 5 | High Dose | 0.139 | 0.142 | 0.021 | 0.021 | 0.160 | 0.163 |

*Gravimetric analysis is performed by weighing the filter papers in the inhalation chamber both before, during and after dosing to calculate the aerosol concentration in the chamber and to determine the duration of dosing.
¹ The targeted and achieved aerosol concentrations quoted assume that the proportion of GLP-1 in the generated atmosphere is 13%. The estimation of total inhaled dose assumed 100% deposition within the respiratory tract.

Whole blood samples (1.4 mL/blood sample) were obtained on Day 5 at the following time points: Pre-dose, 10, 30, 45, 60, 90, 120 minutes and 4 hours post-dose. Blood was collected via venipuncture from the femoral vein. Blood samples were divided into 2 aliquots; one for plasma GLP-1 analysis (0.8 mL) and the other (0.6 mL) for serum FDKP analysis. For plasma GLP-1 analysis, at each timepoint, the whole blood (0.8 mL) was collected into 1.3 mL EDTA tubes (0.1% EDTA). DPP-IV inhibitor (Millipore—Billerica, Mass.) was added (10 μL/mL of blood) to the tubes approximately 5-10 seconds after blood collection (yielding a concentration of DPP-IV of 100 μM). Tubes were inverted several times and immediately placed onto wet ice. Whole blood samples were maintained on wet ice until centrifuged, (2°-8° C.) at 4000 rpm for approximately 10 minutes, to produce plasma. Plasma samples were transferred into appropriate vials and maintained on dry ice prior to storage in a freezer at −70 (±10) ° C. Plasma concentrations (Cₘₐₓ), Tₘₐₓ, AUC, and T_{1/2} were determined for GLP-1.

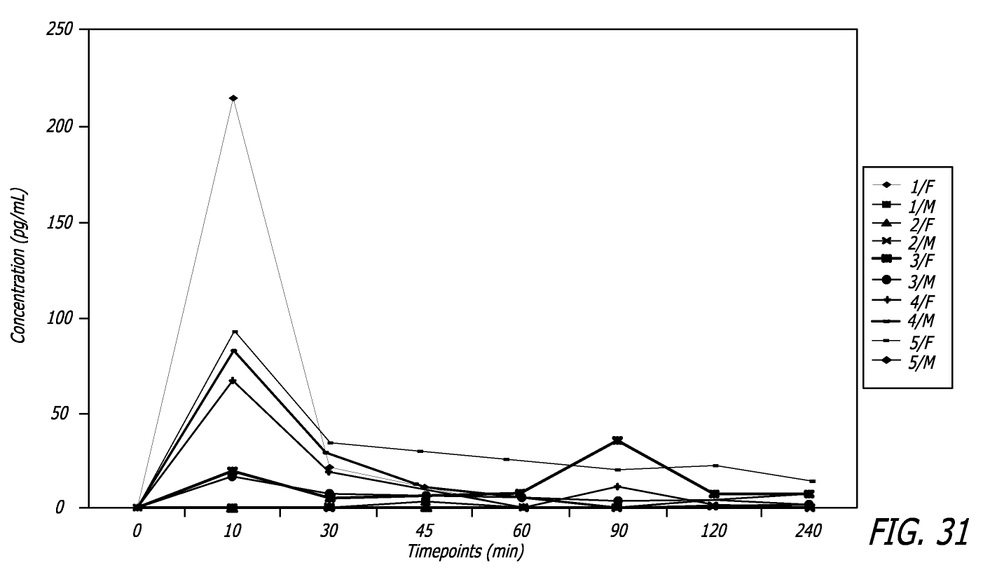

After inhalation administration of GLP-1/FDKP for four consecutive days, detectable levels of GLP-1 were found in all pre-dose samples on Day 5. On Day 5, peak plasma concentrations (Cₘₐₓ) of GLP-1 were achieved within approximately 10 minutes following dose administration (FIG. 31).

Dose related increases in GLP-1 Cₘₐₓ and AUCₗₐₛₜ (area under the concentration-time curve from time zero to the time of the last quantifiable concentration) as a function of the dose were observed in both male and female monkeys on Day 5. Over the dose range studied, less than dose proportional increases in GLP-1 AUCₗₐₛₜ were observed with increasing doses in both male and female monkeys, except for males at the 1 mg/kg/day dose level. A 6.7 fold increase in dose from 0.3 to 2.0 mg/kg/day only resulted in a 2.9 fold increase in AUCₗₐₛₜ in males and 1.1 fold increase in AUCₗₐₛₜ in females.

The peak concentration of GLP-1 averaged 17.2, 93.1 and 214 pg/mL in males and 19.3, 67.9 and 82.8 pg/mL in females when administered GLP-1/FDKP at dose levels of 0.3, 1.0 and 2.0 mg/kg/day respectively. Plasma levels of GLP-1 declined rapidly with apparent elimination half-lives ranging from 4 minutes to 24 minutes.

The AUC values for GLP-1 were 21.6, 105 and 62.3 pg*h/nL in males and 33.4 23.7 and 35.4 pg*h/nL in females when administered GLP-1/FDKP at dose levels of 0.3, 1.0 and 2.0 mg/kg/day respectively.

There were no apparent gender differences in TK parameters of GLP-1 observed at the lowest dose level. However, male monkeys displayed consistently higher AUCₗₐₛₜ values than female monkeys at the mid and high dose levels investigated. Some samples from the vehicle control and control (air) monkeys showed measurable levels of GLP-1. This may have been caused by the contamination of the air inhaled by the animals or may have been a measure of endogenous GLP-1 in those particular monkeys. It should be noted that control animals were exposed in different rooms to the GLP-1/FDKP treated animals.

Since the biological half-life of GLP-1 is less than 15 minutes, the GLP-1 from the administration of GLP-1/FDKP should be completely eliminated within 24 hours. Therefore, endogenous levels of GLP-1 were the likely explanation for consistently quantifiable levels of GLP-1 in time zero samples collected on Day 5 in all GLP-1/FDKP treated animals. Subtracting the time zero values from the observed concentrations of GLP-1 post dosing should reflect the change in GLP-1 due to the administration of GLP-1/FDKP.

For serum FDKP analysis, at each timepoint, the whole blood (0.6 mL) was collected into tubes containing no anticoagulant, allowed to clot at room temperature for a minimum of 30 minutes and separated by centrifugation to obtain serum. FDKP analysis and serum concentrations (Cₘₐₓ), Tₘₐₓ, AUC, and T_{1/2}) were determined. After inhalation administration of GLP-1/FDKP for four consecutive days, detectable levels of FDKP were found in all post-dose samples on Day 5. On Day 5, peak plasma concentrations (Cₘₐₓ) of FDKP were achieved approximately 10 to 30 minutes following dose administration.

There was a dose related increase in FDKP AUC∞ (area under the concentration-time curve from time zero extrapolated to the infinite time), as a function of the dose, observed in both male and female monkeys on Day 5. However, in females there was no difference in FDKP AUC∞, between 0.3 and 1.0 mg/kg/day but a dose related increase was noted between 1 and 2 mg/kg/day. In all instances where an increase was observed, it was less than dose proportional. A 6.7 fold increase in dose from 0.3 to 2.0 mg/kg/day resulted in a 2.7 fold increase in AUCₗₐₛₜ in males and 3.0 fold increase in AUC∞ in females. The peak concentration (Cₘₐₓ) of FDKP averaged 200, 451 and 339 ng/mL in males and 134, 161 and 485 ng/mL in females administered GLP-1F/DKP at dose levels 0.3, 1.0 and 2.0 mg/kg/day respectively. The AUC∞ values for FDKP were 307, 578 and 817 ng.h/mL in males and 268, 235 and 810 ng.h/mL in females administered GLP-1/FDKP at dose levels of 0.3, 1.0 and 2.0 mg/kg/day respectively. AUC∞ and Cₘₐₓ levels in animals administered FDKP only at a dose of 2.1 mg/kg/day (Group 2) were of the same order of magnitude as animals receiving GLP-1/FDKP at 2.13 mg/kg/day, with the exception that the Tₘₐₓ was slightly longer at 30 to 45 minutes following dose administration.

Overall, GLP-1/FDKP was well tolerated with no clinical signs or effects on body weights, food consumption, clinical pathology parameters, macroscopic or microscopic evaluations. It is also noted that inhalation administration of GLP-1/FDKP to cynomolgus monkeys at estimated achieved doses of up to 2.13 mg/kg/day (corresponding to a dose of 0.26 mg/kg/day GLP-1) administered for 30 minutes a day for 5 days is not associated with any dose limiting toxicity.

### Example 15

### Toxicokinetics of GLP-1/FDKP Administered for 14 Days Via Pulmonary Insufflation in Rats

This study evaluated the potential toxicity of GLP-1/FDKP after daily administration via pulmonary insufflation for 14 consecutive days. Rats received control (air), FDKP particles at 10 mg/kg, or 1 (0.15 mg GLP-1), 3 (0.45 mg GLP-1) or 10 (1.5 mg GLP-1) mg/kg GLP-1/FDKP as a daily pulmonary insufflation for 14 consecutive days (n=24/sex/group). Animals were observed daily for clinical signs of toxicity; body weight and food consumption were also recorded.

On Days 1 and 14, GLP-1 Cₘₐₓ was achieved within approximately 10 to 15 minutes following dose administration in all dose groups. Peak concentrations of GLP-1 at 10 mg/kg/day GLP-1/FDKP averaged 6714 and 6270 pg/mL on Day 1 and 2979 and 5834 pg/mL on Day 14 in males and females, respectively. Plasma levels of GLP-1 declined with apparent elimination half-lives ranging from 0.7 hours to 4.4 hours. Mean AUC levels of GLP-1 were 2187 pM*h in males and 2703 pM*h in females at the highest dose of 10 mg/kg/day GLP-1/FDKP. Minimal or no accumulation of GLP-1 was observed and there were no gender differences in Cₘₐₓ, half-life and Tₘₐₓ. AUC values of GLP-1 were slightly higher in female rats than in male rats across all doses. The No Observable Adverse Effect Level (NOAEL) in rats administered GLP-1/FDKP for 14 consecutive days via pulmonary insufflation was 10 mg/kg/day GLP-1/FDKP (1.5 mg/kg/day GLP-1).

Approximately 24 hours after the final dose, animals (12/sex/group) were sacrificed; clinical pathology, macroscopic and microscopic evaluations were performed. The toxicokinetic (TK) satellite animals (12/sex/group) were sacrificed on Day 14 of dosing after the final blood collection. There were no deaths or clinical observations related to GLP-1/DKP. There were no differences in body weights or in food consumption between control and treated animals. At 10 mg/kg GLP-1/FDKP in females only, liver weights and liver to body weight ratios were significantly lower compared to the control (air) group.

There were no clear differences noted from the results for hematology, coagulation, chemistry, urinalysis, or urine chemistry between rats administered vehicle and air controls. There were no gross or histopathological findings in tissues that were determined to have potential toxicity due to administration of GLP-1/FDKP.

### Example 16

### Toxicokinetics of GLP-1/FDKP Administered for 28 Days Via Pulmonary Insufflation in Monkeys

This study evaluated toxicity and toxicokinetics of GLP-1/FDKP administered daily via inhalation for at least 4 weeks. To assess the reversibility, persistence or delayed occurrence of any effects, there was a 4-week recovery period.

Animals received one of the following treatments: Group 1: control (air); Group 2: 3.67 mg/kg/day FDKP particles; Group 3: 0.3 mg/kg/day GLP-1/FDKP (0.045 mg/kg/day GLP-1); Group 4: 1 mg/kg/day GLP-1/FDKP (0.15 mg/kg/day GLP-1) or Group 5: 2.6 mg/kg/day GLP-1/FDKP (0.39 mg/kg/day GLP-1). Forty-two cynomolgus monkeys were divided into 2 groups: main study (n=3/sex/group) and recovery (n=2/sex/group) in groups 1, 2, and 5. Group 1: air control Group 2: FDKP (4 mg/kg/day); Group 3: 0.3 mg/kg/day GLP-1/FDKP (low dose); Group 4: 0.0 mg/kg/day GLP-1/FDKP (mid dose); Group 5: 2.6 mg/kg/day GLP-1/FDKP (high dose). As is typically, in monkey studies only the high dose and controls were evaluated at recovery.

Animals were observed twice daily for mortality and morbidity and at least once daily, 30 minutes post-dose, for abnormalities and signs of toxicity. Body weight data was collected weekly and qualitative food consumption was assessed daily. Blood was collected for toxicokinetics on Days 1, 28, and 56. Three animals/sex/group were anesthetized, weighed, exsanguinated, and necropsied on Day 29. The remaining animals in Groups 1, 2 and 5 (n=2/sex/group) were anesthetized, weighed, exsanguinated, and necropsied on Day 57. At necropsy, selected organs were weighed and selected tissues were collected and preserved. All tissues from each animal were examined microscopically.

There were occasional fluctuations in body weight across all groups; however, there was no treatment related effect on body weight. Generally, all animals maintained or gained minor amounts of weight over the course of the study. Higher incidence and frequency of loose or liquid feces was observed at high doses. There were no significant changes noted in any clinical chemistry parameters that were considered to be treatment-related with the exception of a moderate increase in lactate dehydrogenase (LDH) and aspartate aminotransferase (AST) in high dose females at Day 29 (the end of treatment); see Table 7. The levels of LDH were also very slightly raised in males. These changes had resolved by the end of the recovery period and were not correlated to any microscopic findings in the liver. The change in AST levels in the high dose female group was primarily due to one out of the five animals.

TABLE 7
Mean % change in ALT, AST and LDH

| | % Change in Mean Value | | |
|---|---|---|---|
| | ALT | AST | LDH |
| Group | (U/L) | (U/L) | (U/L) |

Females

| | | | |
|---|---|---|---|
| 1. Control | −2 | 52 | −9 |
| 2. 3.67 mg/kg/day FKDP | −13 | −34 | −53 |
| 3. 0.3 mg/kg/day GLP-1/FKDP | −11 | 53 | −14 |
| 4. 1.0 mg/kg/day GLP-1/FKDP | −15 | 9 | −11 |
| 5. 2.6 mg/kg/day GLP-1/FKDP | 32 | 422 | 117 |

Males

| | | | |
|---|---|---|---|
| 1. Control | −16 | −42 | −62 |
| 2. 3.67 mg/kg/day FKDP | 14 | 60 | −6 |
| 3. 0.3 mg/kg/day GLP-1/FKDP | 24 | 168 | 69 |
| 4. 1.0 mg/kg/day GLP-1/FKDP | 49 | 32 | 7 |
| 5. 2.6 mg/kg/day GLP-1/FKDP | −16 | 30 | 6 |

There was no evidence of any treatment-related macroscopic or histological changes at dose levels up to 2.6 mg/kg/day GLP-1/FDKP. GLP-1/FDKP was well tolerated with no significant clinical signs or effects on body weights, food consumption, hematology, urinalysis, insulin analysis, opthalmoscopy, ECG, macroscopic or microscopic changes observed in doses up to 2.6 mg/kg/day GLP-1/FDKP (0.39 mg/kg/day GLP-1). Inhalation administration of FDKP at an estimated achieved dose of up to 3.67 mg/kg/day for 28 days for up to 30 minutes a day was also not associated with any toxicity.

Dose related increases in GLP-1 and FDKP Cₘₐₓ and AUCₗₐₛₜ as a function of dose were observed in both male and female monkeys on Day 1. Over the dose range studied, less than dose-proportional increases in GLP-1 Cₘₐₓ but not AUCₗₐₛₜ were observed with increasing doses in both male and female monkeys on Day 28. Peak concentrations of GLP-1 at 2.6 mg/kg/day GLP-1/FDKP averaged 259 pg/mL in males and 164 pg/mL in females. Plasma levels of GLP-1 declined with elimination half lives varying from 0.6 to 2.5 hours. Mean AUC values for GLP-1 were 103 pg*hr/nL in males and 104 pg*hr/mL in females at the high dose. Female monkeys displayed higher AUC and Cₘₐₓ values at the low dose compared to males. Peak concentrations of FDKP at 2.6 mg/kg/day GLP-1/FDKP averaged 1800 ng/mL in males and 1900 pg/mL in females.

In conclusion, inhalation administration of GLP-1/FDKP to cynomolgus monkeys at estimated achieved doses of up to 2.6 mg/kg/day GLP-1/FDKP or 0.39 mg/kg/day GLP-1 administered for 28 days for up to 30 minutes a day was clinically well tolerated. The NOAEL was 2.6 mg/kg/day GLP-1/FDKP (0.39 mg/kg/day GLP-1). As described in Example 19 below, the maximum human dose in the Phase I study will be 1.5 mg GLP-1/FDKP per day or 0.021 mg/kg GLP-1 (assuming 70 Kg human). Additional studies will dose to 3.0 mg GLP-1/FDKP per day or ˜0.042 mg/kg GLP-1.

### Example 17

### Preparation of Exendin/FDKP Formulations

Exendin-4/FDKP was prepared by combining an acidic exendin-4 peptide (SEQ ID No. 3) solution with a FDKP particle suspension. The acidic peptide solution was 10% (w/w) of peptide dissolved in 2% acetic acid. The FDKP suspension contained approximately 10% (w/w) FDKP particles. The acidic exendin-4 peptide solution was added to the FDKP particle suspension as it gently mixed. The exendin-4/FDKP mixture was slowly titrated with a 25% ammonia solution to pH 4.50. The mixture was then pelleted into liquid nitrogen and lyophilized.

The % Respirable Fraction on Fill (% RF on Fill) contents for a 15% Exendin-4/FDKP powder was 36%, with a Percent Cartridge Emptying of 99%. A 15% GLP-1/FDKP powder produced at a similar scale showed a % RF on Fill contents of 34%, with a Percent Cartridge Emptying of 100%.

### Example 18

### Pharmacokinetics of Exendin/FDKP Administered Via Pulmonary Insufflation

Repeat dose preliminary toxicity studies to examine the pharmacodynamic and pharmacokinetics profile of exendin-4 (a GLP-1 analogue) in an exendin-4/FDKP formulation at various concentrations, and after multiple administrations via the pulmonary route are in progress.

Twenty-eight day studies in rats and monkeys are performed. Exendin/FDKP is dosed daily, via the inhalation route. In studies where animals are dosed for 28 days, a proportion of the animals are sacrificed immediately after the dosing regimen while other animals are allowed up to a one month recovery period prior to sacrifice. All animals are evaluated for clinical signs of toxicity; various physiological parameters including blood levels of Exendin-4, glucose, and insulin; organ weights, and clinical pathology and histopathology of various organs.

The initial study groups consisted of five animals per group with two control groups: air and Exendin administered intravenously. There were six pulmonary insufflation groups which received approximately 2.0 mg doses of Exendin/FDKP at 5%, 10%, 15%, 20% and 25%, and 30% Exendin load (w/w). Whole blood was collected for blood glucose and Exendin concentrations out to an 8 hour time point.

The data (Cₘₐₓ, T_{1/2} and Tₘₐₓ), are collected, demonstrating that Exendin/FDKP formulations have comparable or better pharmacokinetics than GLP-1/FDKP.

### Example 19

### Pharmacokinetics of GLP-1/xDKP Administered Via Pulmonary Insufflation in Rats

To determine whether different DKPs may influence the pharmacokinetic profile of GLP-1/FDKP formulations, various GLP-1/xDKP formulations were made as disclosed in U.S. Provisional Patent Application entitled “Asymmetrical FDKP Analogs for Use as Drug Delivery Agents” filed on even date herewith and incorporated herein in its entirety (Atty Docket No. 51300-00041).

Studies were conducted in rats divided into 6 treatment groups consisting of five animals per group. The control group (n=3) received GLP-1 via liquid instillation. GLP-1/FDKP (0.3 mg GLP-1), administered by pulmonary insufflation, was also used as a second control. Each of the GLP-1/xDKP treated groups received GLP-1/xDKP formulations via pulmonary insufflation at ˜2.0 mg doses of xDKP loaded with GLP-1 at 10% and 15%. The xDKPs used were (E)-3-(4-(3,6-dioxopiperazin-2-yl)butylcarbamoyl)-acrylic acid), (3,6-bis(4-carboxypropyl)amidobutyl-2,5-diketopiperazine), and ((E)-3,6-bis(4-(Carboxy-2-propenyl)amidobutyl)-2,5-diketopiperazine disodium salt) loads. Whole blood was collected for evaluation of GLP-1 concentrations at 5, 10, 20, 30, 45, 60 and up to 90 minutes post dose.

### Example 20

### A Phase 1a, Single-Dose, Open-Label, Ascending Dose, Controlled Safety and Tolerability Trial of GLP-1/FDKP Inhalation Powder in Healthy Adult Male Subjects

GLP-1 has been shown to control elevated blood glucose in humans when given by intravenous (iv) or subcutaneous (sc) infusions or by multiple subcutaneous injections. Because of the extremely short half-life of the hormone, continuous subcutaneous infusion or multiple daily subcutaneous injections would be required. Neither of these routes is practical for prolonged clinical use. Experiments in animals showed that when GLP-1 was administered by inhalation, therapeutic levels could be achieved.

Several of the actions of GLP-1, including reduction in gastric emptying, increased satiety, and suppression of inappropriate glucagon secretion appear to be linked to the burst of GLP-1 released as meals begin. By supplementing this early surge in GLP-1 with GLP-1/FDKP inhalation powder a pharmacodynamic response in diabetic animals can be elicited. In addition, the late surge in native GLP-1 linked to increased insulin secretion can be mimicked by post-prandial administration of GLP-1/FDKP inhalation powder.

The Phase 1a clinical trial of GLP-1/FDKP inhalation powder is designed to test the safety and tolerability of selected doses of a new inhaled glycemic control therapeutic product for the first time in human subjects. Administration makes use of the MedTone® Inhaler device, previously tested. The primary intent of this clinical trial is to identify a range of doses for GLP-1/FDKP inhalation powder by pulmonary inhalation that are safe, tolerable and can be used in further clinical trials to establish evidence of efficacy and safety. The doses selected for the phase 1a clinical trial are based on animal safety results from non-clinical trials of GLP-1/FDKP inhalation powder described in above Examples, in rats and primates.

Twenty-six (26) subjects are enrolled into 5 cohorts achieve up to 4 evaluable subjects in each of cohorts 1 and 2 and up to 6 evaluable subjects in each of cohorts 3 to 5 who meet eligibility criteria and complete the clinical trial. Each subject is dosed once with Glucagon-Like Peptide-1 (GLP-1) as GLP-1/FDKP Inhalation Powder at the following dose levels: cohort 1: 0.05 mg; cohort 2: 0.45 mg; cohort 3: 0.75 mg; cohort 4: 1.05 mg and cohort 5: 1.5 mg of GLP-1. Dropouts will not be replaced. These dosages assume a body mass of 70 kg. Persons of ordinary skill in the art can determine additional dosage levels based on the studies disclosed above.

The objectives of this trial are to determine the safety and tolerability of ascending doses of GLP-1/FDKP inhalation powder in healthy adult male subjects. The tolerability of ascending doses of GLP-1/FDKP inhalation powder as determined by monitoring pharmacological or adverse effects on variables, including reported adverse events (AE), vital signs, physical examinations, clinical laboratory tests and electrocardiograms (ECG) will be evaluated.

The secondary objectives are to evaluate additional safety and pharmacokinetic parameters. These include additional safety parameters, as expressed by the incidence of pulmonary and other AEs and changes in pulmonary function between Visit 1 (Screening) and Visit 3 (Follow-up); pharmacokinetic (PK) parameters of plasma GLP-1 and serum fumaryl diketopiperazine (FDKP) following dosing with GLP-1/FDKP inhalation powder, as measured via AUC₀₋₁₂₀₍ₘᵢₙ₎ plasma GLP-1 and AUC_{0-480 min} serum FDKP; and additional PK parameters of plasma GLP-1 include: tₘₐₓ plasma GLP-1; Cₘₐₓ plasma GLP-1; and T_{1/2} plasma GLP-1. Additional PK parameters of serum FDKP include: Tₘₐₓ serum FDKP; Cₘₐₓ serum FDKP; and T_{1/2} serum FDKP.

Trial Endpoints are based on a comparison of the following pharmacological and safety parameters determined in the trial subject population. Primary endpoints will include: Safety endpoints will be assessed based on the incidence and severity of reported AEs, including cough and dyspnea, nausea and/or vomiting, as well as changes from screening in vital signs, clinical laboratory tests and physical examinations. Secondary endpoints will include: PK disposition of plasma GLP-1 and serum FDKP (AUC_{0-120 min} plasma GLP-1 and AUC_{0-480 min} serum FDKP); additional PK parameters of plasma GLP-1 (Tₘₐₓ plasma GLP-1, Cₘₐₓ plasma GLP-1 T_{1/2} plasma GLP-1; additional PK parameters of serum FDKP (Tₘₐₓ serum FDKP, Cₘₐₓ serum FDKP); and additional safety parameters (pulmonary function tests (PFTs)) and ECG.

The Phase 1a, single-dose trial incorporates an open-label, ascending dose structure and design strategy that is consistent with 21 CFR 312, Good Clinical Practice: Consolidated Guidance (ICH-E6) and the Guidance on General Considerations for Clinical Trials (ICH-E8) to determine the safety and tolerability of the investigational medicinal product (IMP).

The clinical trial will consist of 3 clinic visits: 1) One screening visit (Visit 1); 2) One treatment visit (Visit 2); and 3) One follow-up visit (Visit 3) 8-14 days after Visit 2. Administration of a single dose of GLP-1/FDKP inhalation powder will occur at Visit 2.

This clinical trial will evaluate safety parameters in each cohort. The cohort scheduled to receive the next dose concentration will not be dosed until a review of all safety and tolerability data for the first or prior doses is conducted by the principal investigator (PI). A half-hour dosing lag time will be implemented between subjects in each cohort to ensure subject safety. The dose may be halted if 3 or more subjects within a cohort, experience severe nausea and/or vomiting or when the maximum dose is reached, or at the discretion of the PI.

Five doses of GLP-1/FDKP inhalation powder (0.05, 0.45, 0.75, 1.05 and 1.5 mg of GLP-1) will be assessed. To accommodate all doses, formulated GLP-1/FDKP will be mixed with FDKP inhalation powder. Single-dose cartridges containing 10 mg dry powder consisting of GLP-1/FDKP inhalation powder (15% weight to weight GLP-1/FDKP) as is or mixed with the appropriate amount of FDKP inhalation powder will be used to obtain the desired dose of GLP-1 (0.05 mg, 0.45 mg, 0.75 mg, 1.05 mg and 1.5 mg).: 1. The first 2 lowest dose levels will be evaluated in 2 cohorts of 4 subjects each and the 3 higher dose levels will be evaluated in 3 cohorts of 6 subjects each. Each subject will receive only 1 dose at 1 of the 5 dose levels to be assessed. In addition to blood draws for GLP-1 (active and total) and FDKP measurements, samples will be drawn for glucagon, glucose, insulin and C-peptide determination.

Numerous references have been made to patents and printed publications throughout this specification. Each of the above-cited references and printed publications are individually incorporated herein by reference in their entirety.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

It is readily apparent to one skilled in the art that various embodiments and modifications can be made to the invention disclosed herein, without departing from the scope and spirit of the invention.

As used herein, the use of the word “a” or “an” when used in conjunction with the term “comprising” in the claims and/or the specification may mean “one,” but it is also consistent with the meaning of “one or more,” “at least one,” and “one or more than one.”

It is contemplated that any method or composition described herein can be implemented with respect to any other method or composition described herein.

The use of the term “or” in the claims is used to mean “and/or” unless explicitly indicated to refer to alternatives only or the alternatives are mutually exclusive.

Throughout this application, the term “about” is used to indicate that a value includes the standard deviation of error for the device or method being employed to determine the value.

Other objects, features and advantages of the present invention will become apparent from the preceeding description and examples as well as the claims. It should be understood, however, that the detailed description and the specific examples, while indicating specific embodiments of the invention, are given by way of illustration only, since various changes and modifications within the spirit and scope of the invention will become apparent to those skilled in the art from this detailed description.

### REFERENCES

The following references, to the extent that they provide exemplary procedural or other details supplementary to those set forth herein, are specifically incorporated herein by reference.
Chelikani P K et al., Intravenous infusion of glucagon-like peptide-1 potently inhibits food intake, sham feeding, and gastric emptying in rats. Am J. Physiol. Regul. Integr. Comp. Physiol., 288(6):R1695-706, 2005.
D'Alessio, et al., J. Clin. Invest., 97:133-38, 1996.
Deacon C F: Therapeutic strategies based on glucagon-like peptide 1. Diabetes. Sep;53(9):2181-9, 2004.
Eissele, et al., Life Sci., 55:629-34, 1994.
Goke, et al., J. Biol. Chem. 268:19650-55, 1993
Johnson J D et al: RyR2 and calpain-10 delineate a novel apoptosis pathway in pancreatic islets. J Biol Chem., 279(23):24794-802, 2004.
Malhotra, R., et al., Regulatory Peptides, 41:149-56, 1992.
Mentlein R, et al., Dipeptidyl peptidase IV hydrolyses gastric inhibitory polypeptide, glucagon-like peptide-1 (7-36) amide, peptide histidine methionine and is responsible for their degradation in human serum. Eur J Biochem., 214:829-835, 1993.
Montrose-Rafizadeh, et al., Diabetes, 45(Suppl. 2):152A, 1996.
Nauck M A, et al., Normalization of fasting hyperglycemia by exogenous GLP-1 (7-36 amide) in type 2 diabetic patients. Diabetologia, 36:741-744, 1993.
Nauck M A, et al., Effects of subcutaneous glucagon-like peptide 1 (GLP-1 [7-36 amide]) in patients with NIDDM. Diabetologia, 39:1546-1553, 1996.
Nauck M A, et al., Effects of glucagon-like peptide 1 on counterregulatory hormone responses, cognitive functions, and insulin secretion during hyperinsulinemic, stepped hypoglycemic clamp experiments in healthy volunteers. J Clin Endocrinol Metab., 87:1239-1246, 2002.
Raufman, et al., J. Biol. Chem. 267:21432-37, 1992.
Raufman, et al., J. Biol. Chem. 266:2897-902, 1991
Schepp, et al., Eur. J. Pharmacol., 69:183-91, 1994.
Singh, et al., Regul. Pept. 53:47-59, 1994.
Sturis J, et al., British Journal of Pharmacology, 140,123. 132, 2003.
Tornusciolo D. R. et al., Biotechniques 19(5):800-805, 1995. Simultaneous detection of TDT-mediated dUTP-biotin nick end-labeling (TUNEL)-positive cells and multiple immunohistochemical markers in single tissue sections.
Verdich C, et al., A meta-analysis of the effect of glucagon-like peptide-1 (7-36) amide on ad libitum energy intake in humans. J Clin Endocrinol Metab., 86:4382-4389, 2001.
Wang Q, et al., Glucagon-like peptide-1 regulates proliferation and apoptosis via activation of protein kinase B in pancreatic INS-1 beta cells. Diabetologia, 47:478-487, 2004.
Wang, et al., J. Clin. Invest., 95:417-21, 1995.
Zander M, et al., Effect of 6-week course of glucagon-like peptide 1 on glycemic control, insulin sensitivity, and beta-cell function in type 2 diabetes: a parallel-group study. Lancet, 359:824-830, 2002.

What is claimed is:

1. A dry powder composition comprising a GLP-1 molecule and a diketopiperazine or a pharmaceutically acceptable salt thereof.

2. The dry powder composition of claim 1, wherein said GLP-1 molecule is selected from the group consisting of native GLP-1s, GLP-1 metabolites, GLP-1 analogs, GLP-1 derivatives, dipeptidyl-peptidase-IV (DPP-IV) protected GLP-1s, GLP-1 mimetics, GLP-1 peptide analogs, or biosynthetic GLP-1 analogs.

3. The dry powder composition of claim 1, wherein said diketopiperazine is a diketopiperazine having the formula 2,5-diketo-3,6-di(4-X-aminobutyl)piperazine, wherein X is selected from the group consisting of succinyl, glutaryl, maleyl, and fumaryl.

4. The dry powder composition of claim 1, comprising a diketopiperazine salt.

5. The dry powder composition of claim 3, wherein said diketopiperazine is 2,5-diketo-3,6-di(4-fumaryl-aminobutyl)piperazine.

6. The dry powder composition of claim 1, wherein said GLP-1 molecule is native GLP-1.

7. The dry powder composition of claim 1, wherein said GLP-1 molecule is an amidated GLP-1 molecule.

8. The dry powder composition of claim 7 wherein the amidated GLP-1 molecule is GLP-1(7-36) amide.

9. A process for forming a particle comprising a GLP-1 molecule and a diketopiperazine comprising the steps of:
providing a GLP-1 molecule;
providing a diketopiperazine in a form selected from particle-forming diketopiperazine, diketopiperazine particles, and combinations thereof, and
combining said GLP-1 molecule and said diketopiperazine in the form of a co-solution,
wherein said particle comprising said GLP-1 molecule and said diketopiperazine is formed.

10. The process of claim 9, further comprising removing a solvent from said co-solution by lyophilization, filtration, or spray drying.

11. The process of claim 10, wherein said particle comprising said GLP-1 molecule and said diketopiperazine is formed by removing said solvent.

12. The process of claim 10, wherein said particle comprising said GLP-1 molecule and said diketopiperazine is formed prior to removing said solvent.

13. The process of claim 9, wherein said GLP-1 molecule is selected from the group consisting of a native GLP-1, a GLP-1 analog, a GLP-1 derivative, a dipeptidyl-peptidase-IV (DPP-IV) protected GLP-1, a GLP-1 mimetic, a GLP-1 peptide analog, or a biosynthetic GLP-1 analog.

14. The process of claim 9, wherein said GLP-1 molecule is provided in the form of a solution comprising a GLP-1 concentration of about 1 g/ml-50 mg/ml.

15. The process of claim 9, wherein said GLP-1 molecule is provided in the form of a solution comprising a GLP-1 concentration of about 0.1 mg/ml-10 mg/ml.

16. The process of claim 9, wherein said GLP-1 molecule is provided in the form of a solution comprising a GLP-1 concentration of about 0.25 mg/ml.

17. The process of claim 9, wherein said diketopiperazine is provided in the form of a suspension of diketopiperazine particles.

18. The process of claim 9, wherein said diketopiperazine is provided in the form of a solution comprising particle-forming diketopiperazine, the process further comprising adjusting the pH of said solution to form diketopiperazine particles.

19. The process of claim 17 or claim 18, further comprising adding an agent to said solution or suspension, wherein the agent is selected from the group consisting of salts, surfactants, ions, osmolytes, chaotropes and lyotropes, acids, bases, and organic solvents.

20. The process of claim 19 wherein said agent promotes association between said GLP-1 molecule and said diketopiperazine particles or said particle-forming diketopiperazine.

21. The process of claim 19 wherein said agent improves the stability or pharmacodynamics of said GLP-1 molecule.

22. The process of claim 19, wherein said agent is sodium chloride.

23. The process of claim 17 or claim 18, further comprising adjusting the pH of said suspension or solution.

24. The process of claim 23, wherein the pH is adjusted to about 4 or greater.

25. The process of claim 9, wherein said GLP-1 molecule in said particle has greater stability.

26. The process of claim 9, wherein said co-solution comprises a GLP-1 concentration of about 1 g/ml-50 mg/ml.

27. The process of claim 9, wherein said co-solution comprises a GLP-1 concentration of about 0.1 mg/ml-10 mg/ml.

28. The process of claim 9, wherein said co-solution comprises a GLP-1 concentration of about 0.25 mg/ml.

29. The process of claim 9, further comprising adding an agent to said co-solution, wherein the agent is selected from the group consisting of salts, surfactants, ions, osmolytes, chaotropes and lyotropes, acids, bases, and organic solvents.

30. The process of claim 29 wherein said agent promotes association between said GLP-1 molecule and said diketopiperazine particles or said particle-forming diketopiperazine.

31. The process of claim 29 wherein said agent improves the stability or pharmacodynamics of said GLP-1 molecule.

32. The process of claim 29, wherein said agent is sodium chloride.

33. The process of claim 9, further comprising adjusting the pH of said co-solution.

34. The process of claim 33, wherein the pH is adjusted to about 4 or greater.

35. A method of administering an effective amount of a GLP-1 molecule to a subject in need thereof said method comprising providing to said subject a particle comprising GLP-1 and diketopiperazine.

36. The method of claim 35, wherein said providing is carried out intravenously, subcutaneously, orally, nasally, buccally, rectally, or by pulmonary delivery.

37. The method of claim 35, wherein said providing is carried out by pulmonary delivery.

38. The method of claim 35, wherein said need comprises the treatment of a condition or disease selected from the group consisting of diabetes, ischemia, reperfused tissue injury, dyslipidemia, diabetic cardiomyopathy, myocardial infarction, acute coronary syndrome, obesity, catabolic changes after surgery, hyperglycemia, irritable bowel syndrome, stroke, neurodegenerative disorders, memory and learning disorders, islet cell transplant and regenerative therapy.

39. The method of claim 35, wherein said provision of said particle results in improved pharmacokinetic half-life and bioavailability of GLP-1 as compared to native GLP-1.

40. A method of forming a powder composition with an improved GLP-1 pharmacokinetic profile, comprising the steps of:
providing a GLP-1 molecule;
providing a particle-forming diketopiperazine in a solution;
forming diketopiperazine particles;
combining said GLP-1 molecule and said solution to form a co-solution; and,
removing solvent from said co-solution by spray-drying to form a powder with an improved GLP-1 pharmacokinetic profile.

41. The method of claim 40, wherein said improved GLP-1 pharmacokinetic profile comprises an increased GLP-1 half-life.

42. The method of claim 41, wherein said increased GLP-1 half-life is greater than or equal to 7.5 minutes.

43. The method of claim 40, wherein said improved GLP-1 pharmacokinetic profile comprises improved bioavailability of GLP-1 as compared to native GLP-1.
